(19) United States
(12) Patent Application Publication
(10) Pub. No.: US 2018/0169641 A1
(43) Pub. Date: Jun. 21, 2018

(54) BENZO[H]QUINOLINE LIGANDS AND COMPLEXES THEREOF

(71) Applicant: JOHNSON MATTHEY PUBLIC LIMITED COMPANY, London (GB)

(72) Inventors: Salvatore BALDINO, Udine (IT); Walter BARATTA, Udine (IT); Andrew BLACKABY, Cambridgeshire (GB); Richard Charles BRYAN, Lancashire (GB); Sarah FACCHETTI, Cambridgeshire (GB); Vaclav JURCIK, Cambridgeshire (GB); Hans Guenter NEDDEN, Cambridgeshire (GB)

(21) Appl. No.: 15/578,054

(22) PCT Filed: Jun. 3, 2016
(86) PCT No.: PCT/GB2016/051657

§ 371 Date: Nov. 29, 2017

(30) Foreign Application Priority Data
Jun. 3, 2015 (GB) 1509663.9

(51) Int. Cl. B01J 31/18; B01J 31/22; C07F 15/00; C07F 15/02; C07D 221/10; C07C 29/14; C07B 41/02

(52) U.S. Cl. CPC B01J 31/189; C07B 31/00; C07F 15/0046; C07F 15/002; C07F 15/02; C07D 221/10; C07C 29/14; C07B 41/02; B01J 2531/0244; B01J 2531/0255; B01J 2531/821; B01J 2531/825; B01J 2531/842; B01J 2231/643; B01J 2231/645; B01J 31/2273

(57) ABSTRACT

The present invention provides substituted tridentate benzo[h]quinoline ligands and complexes thereof. The invention also provides the preparation of the ligands and the respective complexes, as well as to processes for using the complexes in catalytic reactions.

The present invention relates to substituted tridentate benzo[h]quinoline ligands and complexes thereof. The invention also relates to the preparation of the ligands and the respective complexes, as well as to processes for using the complexes in catalytic reactions.

WO2009/007443 (to the Universitá degli Studi di Udine) describes a class of compounds derived from benzo[h]quinoline comprising a —CHR₁—NH₂ group in position 2. WO2009/007443 describes the synthesis of HCNN—H, HCNN-Me and HCNN^{t}Bu but does not describe the compounds, ligands or complexes of the present invention.

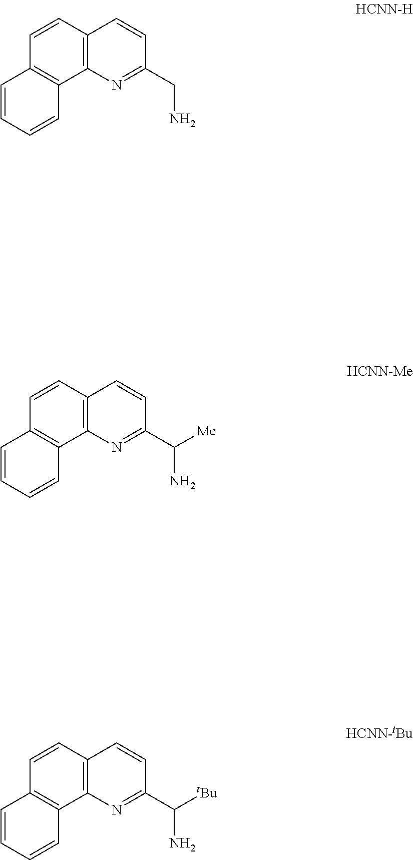 [SMILES: NCc1ccc2ccc3ccccc3c2n1 ; CC(N)c1ccc2ccc3ccccc3c2n1 ; CC(C)(C)C(N)c1ccc2ccc3ccccc3c2n1]

The present inventors have developed substituted tridentate benzo[h]quinoline ligands and complexes thereof. The processes for the preparation of the ligands overcome problems associated with the prior art. The processes are more suited to large-scale manufacture of the ruthenium complexes.

### SUMMARY OF THE INVENTION

In one aspect, the present invention provides a benzo[h]quinoline compound of formula (1a) or (1b), or salts thereof:

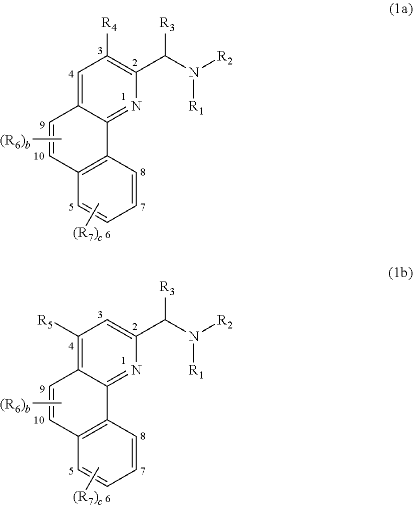

wherein R₁, R₂, R₃, R₄, R₅, R₆, R₇, b and c are as defined herein.

In another aspect, the invention provides a process for preparing a compound of formula (1a) or (1b), the process comprising the step of reacting a compound (4a) or (4b) with a base and a compound of formula (5):

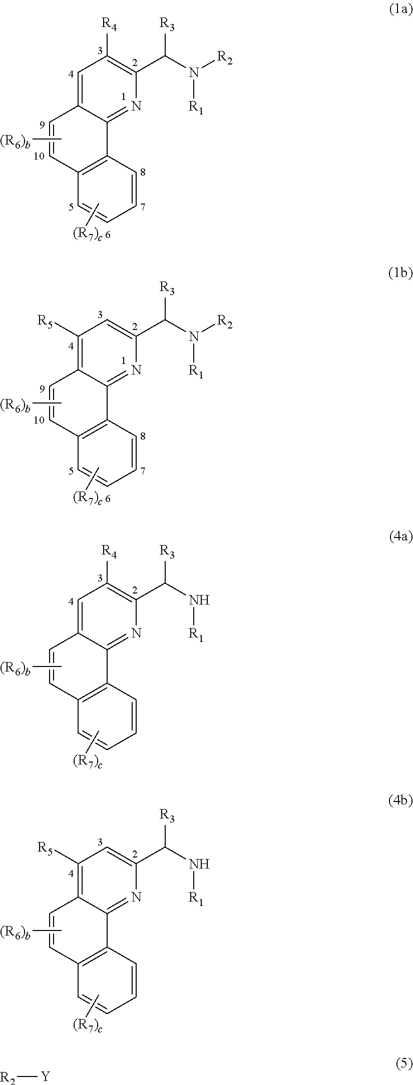

wherein R₁, R₂, R₃, R₄, R₅, R₆, R₇, b, c and Y are as defined herein.

In another aspect, the invention provides a compound which is selected from the compounds of formulae (4a), (4b), (6a), (6b), (7a), (7b), (9a), (9b), (12a), (12b), (13a), (13b), (20a) or (20b).

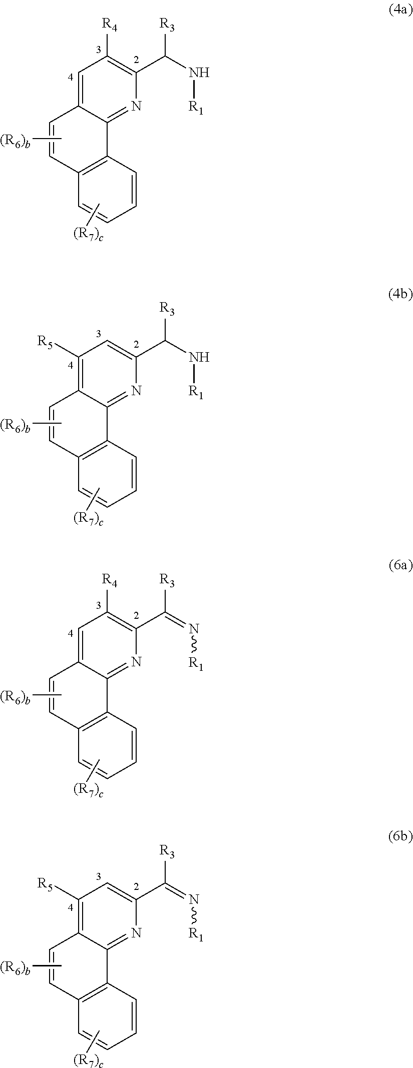

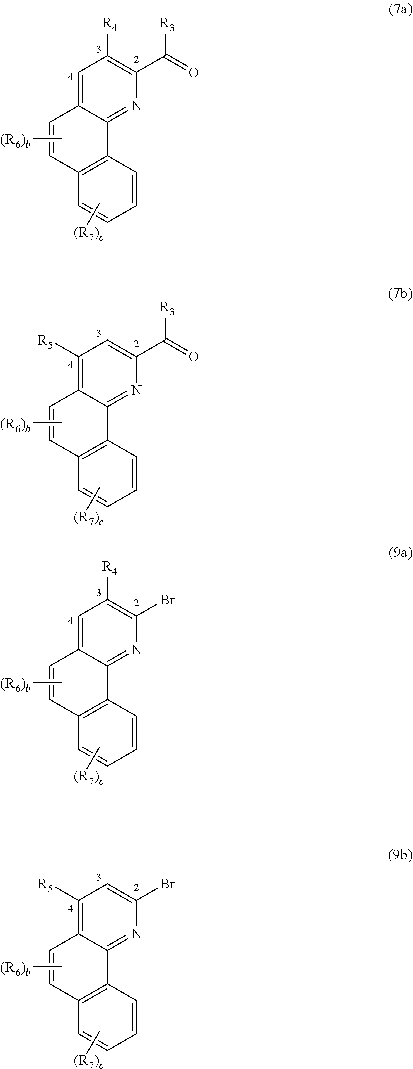

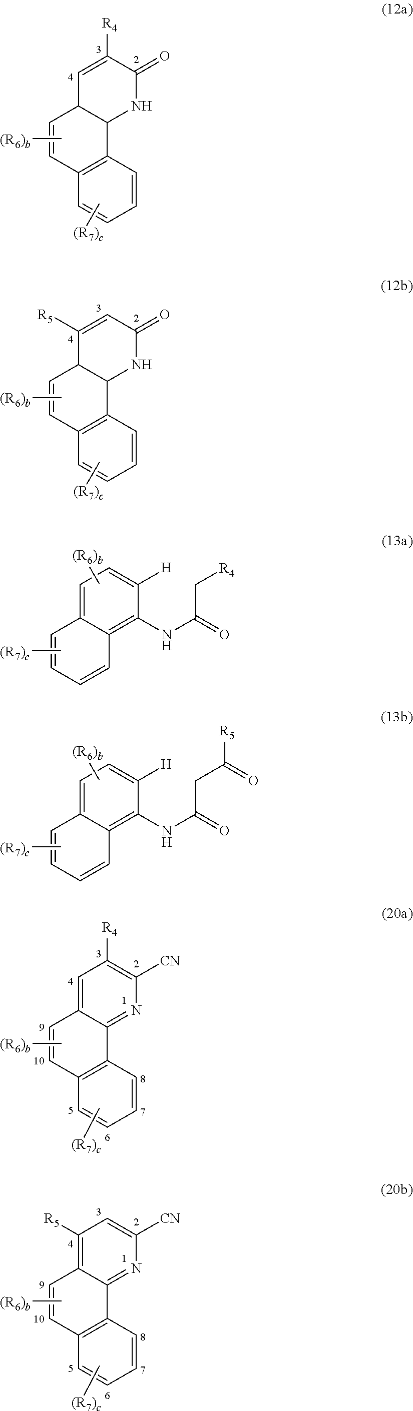

wherein R₁, R₃, R₄, R₅, R₆, R₇, b and c are as defined herein.

In another aspect, the invention provides a transition metal complex of formula (3):

[MX(L¹)ₘ(L²)] (3)

wherein:

M is ruthenium, osmium or iron;
X is an anionic ligand;
L¹ is a monodentate phosphorus ligand, or a bidentate phosphorus ligand;
m is 1 or 2, wherein,
when m is 1, L¹ is a bidentate phosphorus ligand;
when m is 2, each L¹ is a monodentate phosphorus ligand; and
L² is a tridentate ligand of formula (2a) or (2b):

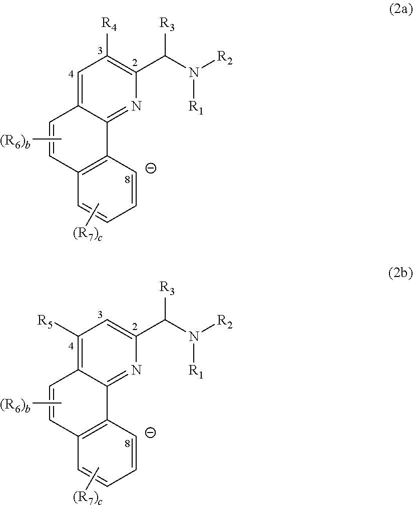

wherein R₁, R₂, R₃, R₄, R₅, R₆, R₇, b and c are as defined herein.

In another aspect, the invention provides a process for preparing a transition metal complex of formula (3) as defined herein, the process comprising the step of reacting a transition metal complex, a ligand L¹, a compound of formula (1a) or (1b) or salts thereof, and a base in an alcohol solvent,

wherein:
the transition metal complex is selected from the group consisting of [ruthenium (arene) (halogen)₂]₂, [ruthenium (halogen) (P(unsubstituted or substituted aryl)₃)], [osmium (arene) (halogen)₂], [osmium (halogen)₂ (P(unsubstituted or substituted aryl)₃)] and [osmium (N(unsubstituted or substituted alkyl)₃)₄ (halogen)₂];

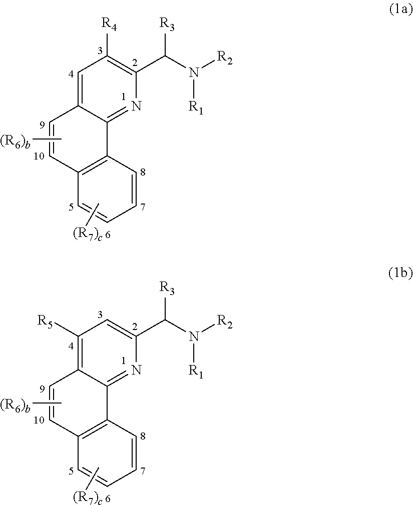

wherein R₁, R₂, R₃, R₄, R₅, R₆, R₇, b and c are as defined herein; and
C-8 of the compound of formula (1a) or (1b) is H.

In another aspect, the invention provides a method of catalysing a reaction, the method comprising the step of reacting a substrate comprising a carbon-oxygen double bond in the presence of a complex of formula (3) as defined herein.

In another aspect, the invention provides a method of catalysing a reaction, the method comprising the step of performing the reaction in the presence of a complex of formula (3) as defined herein, wherein the reaction is selected from the group consisting the isomerization of allylic alcohols, dehydrogenation reactions, the reduction of the alkenyl bond in α,ß-unsaturated carbonyls and in “hydrogen borrowing” reactions.

### Definitions

The point of attachment of a moiety or substituent is represented by “—”. For example, —OH is attached through the oxygen atom.

“Alkyl” refers to a straight-chain or branched saturated hydrocarbon group. In certain embodiments, the alkyl group may have from 1-20 carbon atoms, in certain embodiments from 1-15 carbon atoms, in certain embodiments, 1-8 carbon atoms. The alkyl group may be unsubstituted. Alternatively, the alkyl group may be substituted. Unless otherwise specified, the alkyl group may be attached at any suitable carbon atom and, if substituted, may be substituted at any suitable atom. Typical alkyl groups include but are not limited to methyl, ethyl, n-propyl, iso-propyl, n-butyl, iso-butyl, sec-butyl, tert-butyl, n-pentyl, n-hexyl and the like.

The term “cycloalkyl” is used to denote a saturated carbocyclic hydrocarbon radical. In certain embodiments, the cycloalkyl group may have from 3-15 carbon atoms, in certain embodiments, from 3-10 carbon atoms, in certain embodiments, from 3-8 carbon atoms. The cycloalkyl group may unsubstituted. Alternatively, the cycloalkyl group may be substituted. Unless other specified, the cycloalkyl group may be attached at any suitable carbon atom and, if substituted, may be substituted at any suitable atom. Typical cycloalkyl groups include but are not limited to cyclopropyl, cyclobutyl, cyclopentyl, cyclohexyl and the like.

“Alkoxy” refers to an optionally substituted group of the formula alkyl-O— or cycloalkyl-O—, wherein alkyl and cycloalkyl are as defined above.

“Aryl” refers to an aromatic carbocyclic group. The aryl group may have a single ring or multiple condensed rings. In certain embodiments, the aryl group can have from 6-20 carbon atoms, in certain embodiments from 6-15 carbon atoms, in certain embodiments, 6-12 carbon atoms. The aryl group may be unsubstituted. Alternatively, the aryl group may be substituted. Unless otherwise specified, the aryl group may be attached at any suitable carbon atom and, if substituted, may be substituted at any suitable atom. Examples of aryl groups include, but are not limited to, phenyl, naphthyl, anthracenyl and the like.

“Arylalkyl” refers to an optionally substituted group of the formula aryl-alkyl-, where aryl and alkyl are as defined above.

“Halo”, “hal” or “halide” refers to —F, —Cl, —Br and —I.

“Heteroalkyl” refers to a straight-chain or branched saturated hydrocarbon group wherein one or more carbon atoms are independently replaced with one or more heteroatoms (e.g. nitrogen, oxygen, phosphorus and/or sulfur atoms). In certain embodiments, the heteroalkyl group may have from 1-20 carbon atoms, in certain embodiments from 1-15 carbon atoms, in certain embodiments, 1-8 carbon atoms. The heteroalkyl group may be unsubstituted. Alternatively, the heteroalkyl group may substituted. Unless otherwise specified, the heteroalkyl group may be attached at any suitable atom and, if substituted, may be substituted at any suitable atom. Examples of heteralkyl groups include but are not limited to ethers, thioethers, primary amines, secondary amines, tertiary amines and the like.

“Heterocycloalkyl” refers to a saturated cyclic hydrocarbon group wherein one or more carbon atoms are independently replaced with one or more heteroatoms (e.g. nitrogen, oxygen, phosphorus and/or sulfur atoms). In certain embodiments, the heterocycloalkyl group may have from 2-20 carbon atoms, in certain embodiments from 2-10 carbon atoms, in certain embodiments, 2-8 carbon atoms. The heterocycloalkyl group may be unsubstituted. Alternatively, the heterocycloalkyl group may be substituted. Unless otherwise specified, the heterocycloalkyl group may be attached at any suitable atom and, if substituted, may be substituted at any suitable atom. Examples of heterocycloalkyl groups include but are not limited to epoxide, morpholinyl, piperadinyl, piperazinyl, thirranyl, pyrrolidinyl, pyrazolidinyl, imidazolidinyl, thiazolidinyl, thiomorpholinyl and the like.

“Heteroaryl” refers to an aromatic carbocyclic group wherein one or more carbon atoms are independently replaced with one or more heteroatoms (e.g. nitrogen, oxygen, phosphorus and/or sulfur atoms). In certain embodiments, the heteroaryl group may have from 3-20 carbon atoms, in certain embodiments from 3-15 carbon atoms, in certain embodiments, 3-8 carbon atoms. The heteroaryl group may be unsubstituted. Alternatively, the heteroaryl group may substituted. Unless otherwise specified, the heteroaryl group may be attached at any suitable atom and, if substituted, may be substituted at any suitable atom. Examples of heteroaryl groups include but are not limited to thienyl, furanyl, pyrrolyl, imidazolyl, pyrazolyl, thiazolyl, isothiazolyl, oxazolyl, isoxazolyl, triazolyl, thiadiazolyl, thiophenyl, oxadiazolyl, pyridinyl, pyrimidyl, benzoxazolyl, benzthiazolyl, benzimidazolyl, indolyl, quinolinyl and the like.

“Substituted” refers to a group in which one or more hydrogen atoms are each independently replaced with substituents (e.g. 1, 2, 3, 4, 5 or more) which may be the same or different. Examples of substituents include but are not limited to -halo, —CF₃, —R^{a}, —O—R^{a}, —S—R^{a}, —NR^{a}R^{b}, —CN, —C(O)—R^{a}, —COOR^{a}, —C(S)—R^{a}, —C(S)OR^{a}, —S(O)₂OH, —S(O)₂—R^{a}, —S(O)₂NR^{a}R^{b} and —CONR^{a}R^{b}, preferably -halo, —CF₃, —R^{a}, —O—R^{a}, —NR^{a}R^{b}, —COOR^{a}, —S(O)₂OH, —S(O)₂—R^{a}, —S(O)₂NR^{a}R^{b} and —CONR^{a}R^{b}. R^{a} and R^{b} are independently selected from the groups consisting of H, alkyl, aryl, arylalkyl, heteroalkyl, heteroaryl, or R^{a} and R^{b} together with the atom to which they are attached form a heterocycloalkyl group, and wherein R^{a} and R^{b} may be unsubstituted or further substituted as defined herein.

### DETAILED DESCRIPTION

### Compounds of Formula (1a) and (1b)

The present invention provides a benzo[h]quinoline compound of formula (1a) or (1 b), or salts thereof:

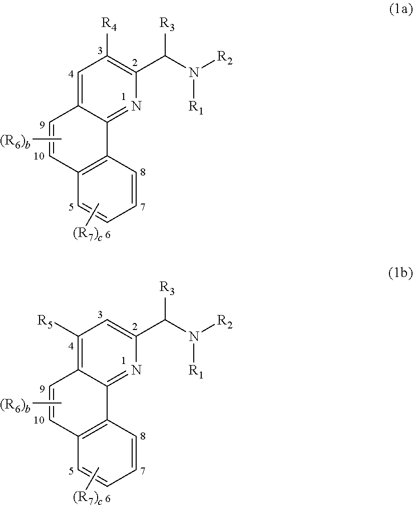

wherein:
R₁ and R₂ are independently selected from the group consisting of —H, —OH, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl;
R₃ is selected from the group consisting of —H, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl;
R₄ is selected from the group consisting of unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, substituted C₅₋₂₀-aryl;
R₅ is selected from the group consisting of unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, substituted C₅₋₂₀-aryl;
R₆ is selected from the group consisting of —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″ and —CONR′R″, wherein R′ and R″ are independently selected from the group consisting of H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, substituted C₇₋₂₀-arylalkyl, or R′ and R″ together with the atom to which they are attached form a substituted or unsubstituted C₂₋₂₀-heterocycloalkyl group;
R₇ is selected from the group consisting of —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″ and —CONR′R″, wherein R′ and R″ are independently selected from the group consisting of H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, substituted C₇₋₂₀-arylalkyl, or R′ and R″ together with the atom to which they are attached form a substituted or unsubstituted C₂₋₂₀-heterocycloalkyl group;
b is an integer selected from 0, 1 or 2; and
c is an integer selected from 0, 1, 2, 3 or 4.

The numbering of the atoms around the benzo[h]quinoline skeleton is illustrated in the formulae above.

The benzo-fused pyridine ring of the compounds of formulae (1) are disubstituted as a group is present at both C-2 and either at C-3 or C-4. The pyridine ring, therefore, may be substituted by a —CH(R₃)—NR₁R₂ amino group at C-2 and group R₄ at C-3 for the compound (1a). In this instance, R₅ is —H. Alternatively, the pyridine ring may be substituted by the —CH(R₃)—NR₁R₂ amino group at C-2 and group R₅ at C-4 for the compound (1b). For this compound, R₄ is —H.

R₁ and R₂ may be independently selected from the group consisting of —H, —OH, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. In one embodiment, R₁ and R₂ are independently selected from the group consisting of —H, —OH, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl and substituted C₅₋₂₀-aryl, such as —H, methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In another embodiment, the alkyl groups may be optionally functionalised with one or more substituents such as halide (—F, —Cl, —Br or —I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl, C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—).

In one embodiment, one of R₁ and R₂ is H and the other is selected from the group consisting of —H, —OH, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. In one preferred embodiment, one of R₁ and R₂ is H and the other is selected from the group consisting of —H, —OH, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl and substituted C₅₋₂₀-aryl, such as —H, —OH, methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally funcationalised with one or more substituents such as halide (—F, —Cl, —Br or —I) or alkoxy groups, e.g. methoxy, ethoxy or propoxy. The aryl group may be optionally functionalised with one or more (e.g. 1, 2, 3, 4, or 5) substituents such as halide (—F, —Cl, —Br or —I), straight- or branched-chain C₁-C₁₀-alkyl, C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—).

In one preferred embodiment, R₁ and R₂ are both —H.

R₃ is selected from the group consisting of —H, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. In one embodiment, R₃ is selected from the group consisting of —H, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl and substituted C₅₋₂₀-aryl, such as —H, methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In another embodiment, the alkyl groups may be optionally substituted with one or more substituents such as halide (—F, —Cl, —Br or —I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl, C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). More preferably, R₃ is selected from —H, methyl, ethyl, n-propyl, iso-propyl, n-butyl, iso-butyl, sec-butyl, tert-butyl and phenyl. In one embodiment, R₃ is —H.

When R₃ is —H, the carbon atom to which R₃ is attached is not chiral. However, when R₃ is not —H, the compounds (1) will contain a chiral centre in the —CH(R₃)—NR₁R₂ group. The compounds (1) can be used as a racemic mixture, as either single enantiomer or as a mixture of enantiomers, preferably as a single enantiomer. The enantiomers of compounds (1) may be obtained in enantiomerically pure form by the resolution of e.g. a racemic mixture of compound (1a) or (1 b).

For the compound (1a), R₄ is selected from the group consisting of unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, substituted C₅₋₂₀-aryl. In one embodiment, R₄ is selected from the group consisting of unsubstituted C₁₋₂₀-alkyl, substituted C₁₋₂₀-alkyl, unsubstituted C₅₋₂₀-aryl, substituted C₅₋₂₀-aryl. In another embodiment, R₄ may be selected from the group consisting of methyl, ethyl, n-propyl, iso-propyl, n-butyl, iso-butyl, sec-butyl, tert-butyl, pentyl, hexyl, heptyl, octyl, nonyl, decyl, dodecyl, stearyl, phenyl, -phenyl-CF₃ (e.g. 2-, 3- or 4-CF₃-phenyl, such as 4-CF₃-phenyl), -pentahalophenyl (e.g. pentafluorophenyl), naphthyl and anthracyl, such as methyl, ethyl, n-propyl, iso-propyl, n-butyl, iso-butyl, sec-butyl, tert-butyl, phenyl, -phenyl-CF₃ (e.g. 2-, 3- or 4-CF₃-phenyl, such as 4-CF₃-phenyl) or -pentahalophenyl (e.g. pentafluorophenyl). In another embodiment, R₄ is selected from the group consisting of unsubstituted C₁₋₂₀-alkyl and unsubstituted C₅₋₂₀-aryl. In another embodiment, R₄ may be selected from the group consisting of methyl, ethyl, n-propyl, iso-propyl, n-butyl, iso-butyl, sec-butyl, tert-butyl, pentyl, hexyl, heptyl, octyl, nonyl, decyl, dodecyl, stearyl, phenyl, naphthyl and anthracyl, such as methyl, ethyl, n-propyl, iso-propyl, n-butyl, iso-butyl, sec-butyl, tert-butyl, phenyl, naphthyl and anthracyl. In one embodiment, R₄ is methyl. In another embodiment, R₄ is phenyl. In another embodiment, R₄ is -phenyl-CF₃. In another embodiment, R₄ is pentafluorophenyl.

For the compound (1 b), R₅ is selected from the group consisting of unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, substituted C₅₋₂₀-aryl. In one embodiment, R₅ is selected from the group consisting of unsubstituted C₁₋₂₀-alkyl, substituted C₁₋₂₀-alkyl, unsubstituted C₅₋₂₀-aryl, substituted C₅₋₂₀-aryl. In another embodiment, R₅ may be selected from the group consisting of methyl, ethyl, n-propyl, iso-propyl, n-butyl, iso-butyl, sec-butyl, tert-butyl, pentyl, hexyl, heptyl, octyl, nonyl, decyl, dodecyl, stearyl, phenyl, -phenyl-CF₃ (e.g. 2-, 3- or 4-CF₃-phenyl, such as 4-CF₃-phenyl), -pentahalophenyl (e.g. pentafluorophenyl), naphthyl and anthracyl, such as methyl, ethyl, n-propyl, iso-propyl, n-butyl, iso-butyl, sec-butyl, tert-butyl, phenyl, -phenyl-CF₃ (e.g. 2-, 3- or 4-CF₃-phenyl, such as 4-CF₃-phenyl) or -pentahalophenyl (e.g. pentafluorophenyl). In another embodiment, R₅ is selected from the group consisting of unsubstituted C₁₋₂₀-alkyl and unsubstituted C₅₋₂₀-aryl. In another embodiment, R₅ may be selected from the group consisting of methyl, ethyl, n-propyl, iso-propyl, n-butyl, iso-butyl, sec-butyl, tert-butyl, pentyl, hexyl, heptyl, octyl, nonyl, decyl, dodecyl, stearyl, phenyl, naphthyl and anthracyl, such as methyl, ethyl, n-propyl, iso-propyl, n-butyl, iso-butyl, sec-butyl, tert-butyl, phenyl, naphthyl and anthracyl. In one preferred embodiment, R₅ is methyl. In another embodiment, R₅ is phenyl. In another embodiment, R₅ is -phenyl-CF₃. In another embodiment, R₅ is pentafluorophenyl. R₆ may be present or absent. When absent, b is 0 i.e. the aryl ring is unsubstituted. When R₆ is present, b may be 1 or 2. When b is 2, each R₆ may be the same or different to each other. The or each R₆ may be selected from the group consisting of —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″ and —CONR′R″, wherein R′ and R″ are independently selected from the group consisting of H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, substituted C₇₋₂₀-arylalkyl, or R′ and R″ together with the atom to which they are attached form a substituted or unsubstituted C₂₋₂₀-heterocycloalkyl group. In one embodiment, R₆ is selected from the group consisting of —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. In one embodiment, R₆ is independently selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl and substituted C₆₋₂₀-aryl, such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In another embodiment, the alkyl groups may be optionally substituted with one or more substituents such as halide (—F, —Cl, —Br or —I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl, C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). In one preferred embodiment, b is 0 i.e. R₆ is absent.

R₇ may be present or absent. When absent, c is 0 i.e. the aryl ring is unsubstituted. When R₇ is present, c may be 1, 2, 3 or 4, such as 1, 2 or 3. When c is 2, 3 or 4, each R₇ may be the same or different to each other. The or each R₇ may be selected from the group consisting of —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₆₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″ and —CONR′R″, wherein R′ and R″ are independently selected from the group consisting of H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, substituted C₇₋₂₀-arylalkyl, or R′ and R″ together with the atom to which they are attached form a substituted or unsubstituted C₂₋₂₀-heterocycloalkyl group. In one embodiment, R₇ is selected from the group consisting of CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₆₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. In one embodiment, R₇ is independently selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl and substituted C₅₋₂₀-aryl, such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In another embodiment, the alkyl groups may be optionally substituted with one or more substituents such as halide (—F, —Cl, —Br or —I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl, C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). In one preferred embodiment, the aromatic ring is unsubstituted at C-8 i.e. R₇ is absent at C-8.

In one preferred embodiment, c is 0 i.e. R₇ is absent.

In another preferred embodiment, c is 1 and is present at C-5. R₆ may be present or absent as described above, preferably, absent i.e. b is 0. The compounds of formula (1a) and (1 b) therefore have the following structures:

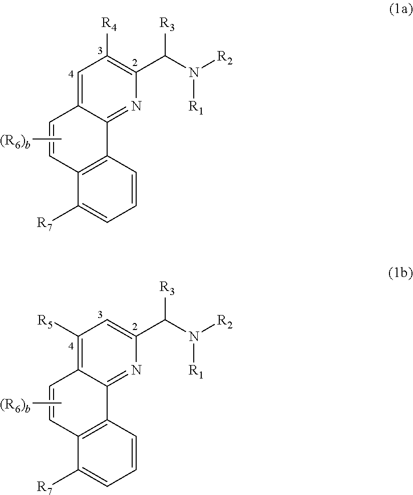

In one preferred embodiment, the compound of formula (1a) may be selected from the group consisting of:

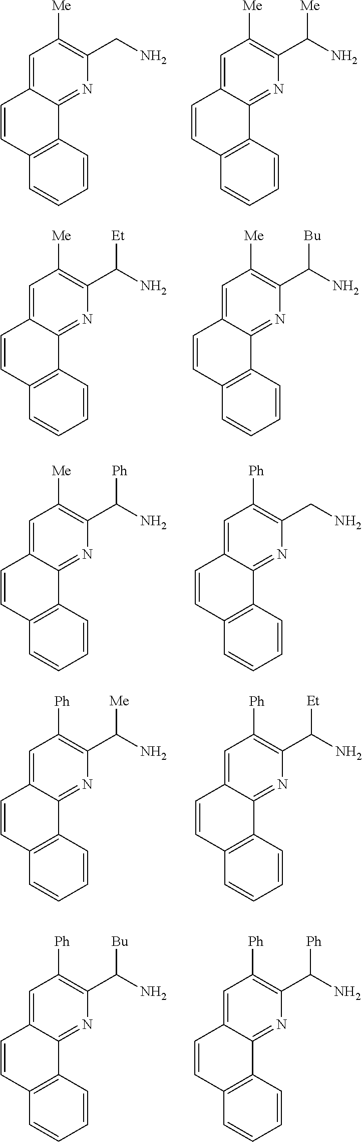

In one particularly preferred embodiment, the compound of formula (1a) may be selected from the group consisting of:

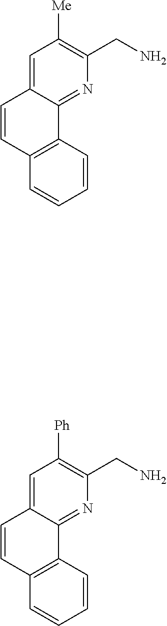 [SMILES: Cc1cc2ccc3ccccc3c2nc1CN ; NCc1nc2c(ccc3ccccc32)cc1-c1ccccc1]

In one preferred embodiment, the compound of formula (1 b) may be selected from the group consisting of:

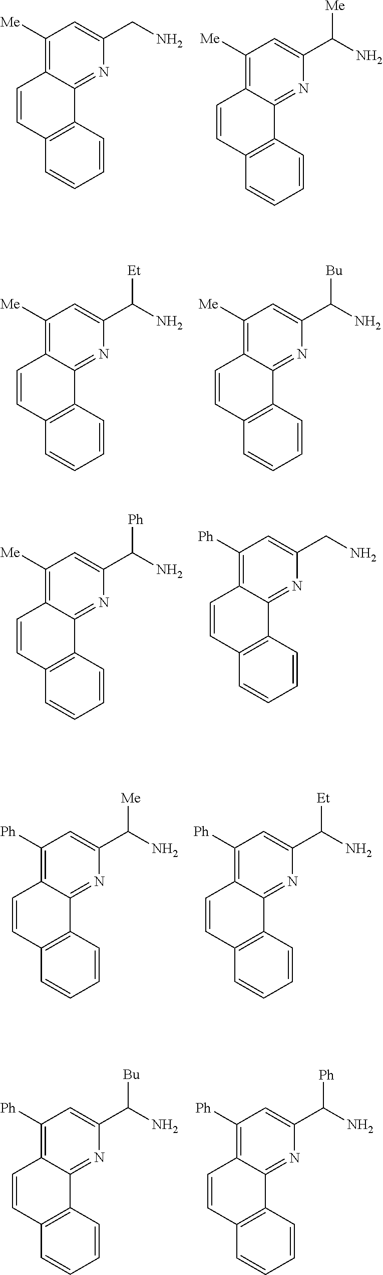

In one particularly preferred embodiment, the compound of formula (1 b) may be selected from the group consisting of:

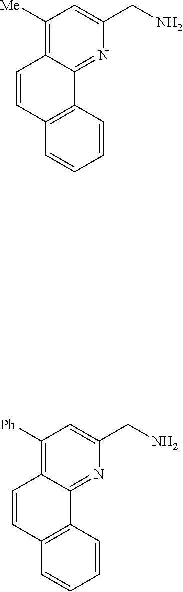 [SMILES: Cc1cc(CN)nc2c1ccc1ccccc12 ; NCc1cc(-c2ccccc2)c2ccc3ccccc3c2n1]

The compounds of formula (1a) and (1b) may form a salt with a suitable acid e.g. a suitable organic or inorganic acid. The compound (1a) or (1b) may be reacted as the free base with a suitable acid to form the salt. Alternatively, the acid may be present in situ during the preparation of the compounds (1a) and (1b). In this instance, the salts of (1a) and (1b) may be isolated directly from the reaction mixture. In one embodiment, the acid may be a hydrohalide acid, such as hydrochloric acid, hydrobromic acid or hydroiodic acid. The salts of compounds (1a) or (1 b) may accordingly be hydrochloride salts, hydrobromide salts or hydroiodide salts. In one embodiment, the salt is a hydrochloride salt. In another embodiment, the acid may be selected from the group consisting of acetic acid, trifluoroacetic acid, methylsulfonic acid, trifluoromethylsulfonic acid, p-toluenesulfonic acid phosphoric acid, benzoic acid, salicylic acid, and citric acid. The salts of compounds (1a) or (1 b) may accordingly be acetate salts, trifluoroacetate salts, methylsulfonate salts, trifluoromethylsulfonate salts, p-toluenesulfonate salts, phosphate salts, benzoate salts, salicylate salts, or citrate salts.

When R₃ of the compound (1a) or (1b) is not —H, optical resolution of the enantiomers of compounds (1a) and (1b) may be performed by methods known in the art. For example, a racemic mixture of compound (1a) may be optically resolved using an acid chiral resolving agent. A racemic mixture of compound (1 b) may be optically resolved likewise. Chiral resolving agents include but are not limited to L-(+)-tartaric acid, D-(−)-tartaric acid, L-(+)-mandelic acid or D-(−)-mandelic acid. It is envisaged that a racemic chiral acid may be used to form a diastereomeric mixture of salts of compounds (1a) and (1b). If desired, resolution of the diastereomers may occur by fractional crystallisation. It is also envisaged that enzymatic resolution of the enantiomers of compounds (1a) and (1 b) may be possible with an enzyme such as a lipase.

The isolation of the compounds (1a) and (1 b) as salts (in particular, hydrochloride salts) provide stable ligand precursors, which can be stored in air at room temperature in the absence of moisture for a long time without degradation (for example, for more than two years) and can be used directly in the preparation of transition metal complexes.

### Preparation of the Compounds of Formula (1a) and (1b)

The compounds of formula (1a) and (1b), and salts thereof, may be prepared from a compound of formula (4a) or (4b), and salts thereof, by methods known in the art. In this respect, a compound (4a) reacts to form a compound (1a) and a compound (4b) reacts to form a compound (1b). For example, the compound (4a) or (4b) may be reacted with a base and a compound of formula (5):

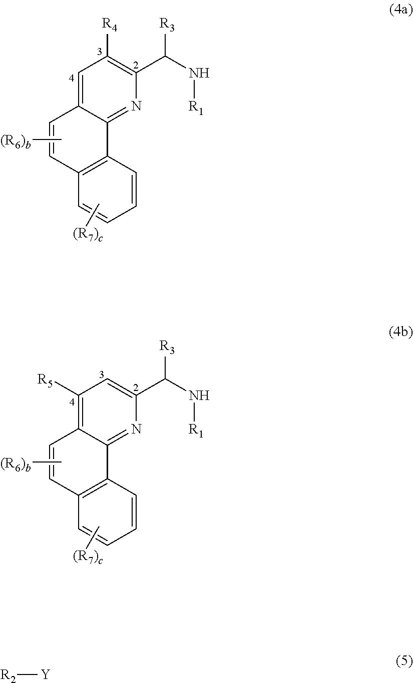

wherein:
Y is a leaving group.
R₁, R₃, R₄, R₅, R₆, R₇, b and c are as generally described above.

In this instance, R₂ may be selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-heteroalkyl, substituted C₁₋₂₀-heteroalkyl, unsubstituted C₂₋₂₀-heterocycloalkyl and substituted C₂₋₂₀-heterocycloalkyl. In one embodiment, R₂ may be selected from the group consisting of unsubstituted C₁₋₂₀-alkyl, substituted C₁₋₂₀-alkyl, unsubstituted C₃₋₂₀-cycloalkyl and substituted C₃₋₂₀-cycloalkyl.

The base may be any suitable base which is capable of deprotonating the —NHR₁ group of the compound (4a) or (4b). Suitable bases include but are not limited to organic or inorganic bases. Inorganic bases may be selected from the group consisting of hydroxides, alkoxides, carbonates, acetates. Suitable hydroxides include alkali metal hydroxides (e.g. lithium hydroxide, sodium hydroxide or potassium hydroxide) or tetraalkylammonium hydroxides (e.g. tetrabutylammonium hydroxide). Suitable alkoxides include alkali metal alkoxides (e.g. lithium alkoxide, sodium alkoxide (such as sodium methoxide) or potassium alkoxide) or tetraalkylammonium alkoxides (e.g tetrabutylammonium hydroxide). Suitable carbonates include but are not limited to potassium carbonate or sodium carbonate. Suitable acetates include but are not limited to potassium acetate or sodium acetate. Organic bases include but are not limited to organolithium reagents, such as butyllithium (e.g. n-, sec- or tert-butyllithium) or lithium diisopropylamide (LDA).

The reaction may be carried out under an inert atmosphere (such as nitrogen or argon). Suitably, a solvent may be used, for example, any suitable protic or aprotic polar solvent or combinations thereof). Suitable protic solvent include but are not limited to alcohols (such as methanol, ethanol, 1-propanol, 2-propanol, 1-butanol, 2-butanol, t-butanol or benzylic alcohol). Suitable aprotic solvents include but are not limited to ethers (e.g. tetrahydrofuran (THF), 2-methyltetrahydrofuran (2-Me-THF), dioxane, methyltertbutylether (MTBE) or diethylether), amides (e.g. dimethylformamide (DMF), N-methylpyrrolidine (NMP) or dimethylacetamide (DMAc)) or chlorinated alkanes (such as chloromethane or dichloromethane (DCM)). The solvent may be anhydrous.

The compound (4a) or (4b), the base, the solvent and the compound (5) may be added in any suitable order. In one embodiment of the invention, however, the compound (4a) or (4b) and the base is placed in a reaction vessel, together with the solvent, and then the compound (5) is added.

Y is a leaving group and may be a halide. In one embodiment, the halide may be selected from the group consisting of chloride, bromide or iodide.

The reaction may be continued for a suitable period of time until it is determined (e.g. by GC) that the reaction substantially complete. The period of time may vary from about 30 minutes to about 72 hours, preferably 30 minutes to about 24 hours. During this time, the reaction temperature may be varied one or more times between about −10° C. and about 25° C. If desired, on completion of the reaction, the compound of formula (1a) or (1b) may be separated from the reaction mixture by any appropriate method.

As described above, the compounds of formula (1a) and (1b) may form a salt with a suitable acid. The compounds (1a) and (1b) may be reacted as the free base with a suitable acid to form the salt. Alternatively, the acid may be present in situ during the preparation of compounds (1a) and (1b). For example, the compounds (4a) and (4b) may be reacted as acid addition salts of compounds (4a) and (4b) forming the acid addition salts of compounds (1a) and (1b). The extra addition of acid to the reaction mixture comprising compounds (4a) and (4b), therefore, may not be necessary in order to prepare salts of compounds (1a) and (1b). The acid used is as generally described above.

### Preparation of the Compounds of Formulae (4a) and (4b)

The compound of formula (4a) or (4b) may be prepared by reducing a compound (6a) or (6b). In this respect, a compound (6a) is reduced to a compound (4a) and a compound (6b) is reduced to a compound (4b).

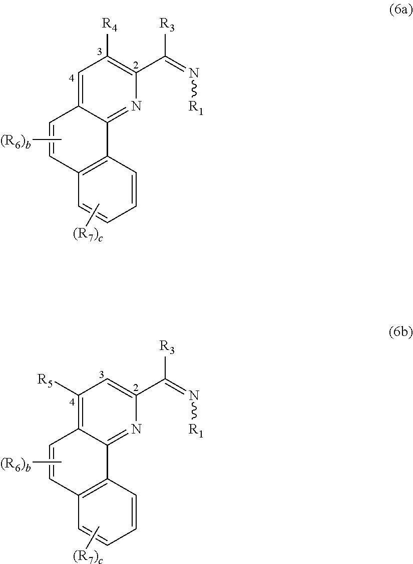

R₁, R₃, R₄, R₅, R₆, R₇, b and c are as generally described above.

It will be understood that, in the depictions herein, where R₁ is connected by a wavy line both or either enantiomer may be present.

In one embodiment, the reduction may be a hydrogenation reaction. The hydrogenation reaction may comprise reacting the compound (6a) or (6b) with gaseous hydrogen in the presence of a hydrogenation catalyst and an acid in a suitable solvent. The hydrogenation catalyst may be a heterogeneous or homogeneous catalyst, preferably a heterogeneous catalyst. The catalyst (whether heterogeneous or homogeneous) should be selected such that the catalyst preferentially reduces the —(R₃)C═N(R₁)— double bond rather than reducing another group present in the compound (6a) or (6b). In one embodiment, the heterogeneous catalyst is a heterogeneous platinum group metal (PGM) catalyst, for example, a heterogeneous palladium or platinum catalyst. In one embodiment, the heterogeneous catalyst is a heterogeneous palladium catalyst. Examples of palladium catalysts include but are not limited to colloidal palladium, palladium sponge, palladium plate or palladium wire. Examples of platinum catalysts include but are not limited to colloidal platinum, platinum sponge, platinum plate or platinum wire.

The heterogeneous PGM metal catalyst may be a PGM on a solid support. The support may be selected from the group consisting of carbon, alumina, calcium carbonate, barium carbonate, barium sulfate, titania, silica, zirconia, ceria and a combination thereof. When the support is alumina, the alumina may be in the form of alpha-Al₂O₃, beta-Al₂O₃, gamma-Al₂O₃, delta-Al₂O₃, theta-Al₂O₃ or a combination thereof. When the support is carbon, the carbon may be in the form of activated carbon (e.g. neutral, basic or acidic activated carbon), carbon black or graphite (e.g. natural or synthetic graphite). An example of a heterogeneous PGM catalyst is palladium on carbon. An example of another heterogeneous PGM catalyst is platinum on carbon.

The catalyst loading may be up to about 20 mole %. A greater catalyst loading may perform the desired reduction, however, increasing the quantity of the PGM may make the process uneconomical. In one embodiment, the catalyst loading may be up to 10 mole % and, in another embodiment, may be in the range of about 0.1-10.0 mole %.

The acid may be any suitable acid, such as a hydrohalide acid e.g. hydrochloric acid, hydrobromic acid or hydroiodic acid. The acid may be added as a reagent to the hydrogenation reaction or the compounds (6a) and (6b) may be reacted as acid addition salts. The salts are as generally described above. Without wishing to be bound by theory, it is believed that the benzo-fused pyridinyl N atom needs to be protonated in order for the hydrogenation to proceed.

Any suitable solvent may be utilised e.g. polar solvents, such as an alcohol. The alcohol may be selected from the group consisting of methanol, ethanol, isopropanol and mixtures thereof. In one embodiment, the solvent is methanol.

The compound (6a) or (6b) may be placed in a pressure vessel together with the hydrogenation catalyst. The pressure vessel may then be assembled and purged with one or more nitrogen/vacuum cycles (e.g. one, two, three or four cycles). The alcohol solvent may then added via the injection port to form a solution of the compound (6a) or (6b), which may have concentration in the range of about 0.01 to about 1 molar, such as about 0.3 molar. If the hydrogenation catalyst is heterogeneous, the catalyst will not dissolve in the alcohol solvent. However, if the hydrogenation catalyst is homogeneous, it may dissolve in the alcohol solvent and form a solution with the compound (5a) or (5b).

Once the alcohol solvent has been added, the pressure vessel may be purged once again with one or more nitrogen/vacuum cycles (e.g. one, two, three, four or five cycles), followed by one or more hydrogen/vacuum cycles (e.g. one, two, three, four or five cycles). During purging the reaction mixture may be agitated (by either stirring or shaking) to encourage removal of dissolved oxygen. The pressure vessel may then be pressurised with hydrogen (e.g. to about 5 bar), stirred and heated to temperature (e.g. about 30° C.). Hydrogen gas uptake may begin after a period of time has elapsed (e.g. after about 45 minutes on a 6 g scale reaction). Once hydrogen uptake begins, the pressure vessel may optionally be depressurised with hydrogen

While it is typically sufficient for a single charge of hydrogenation catalyst to be added to the reaction mixture, a second or further charge may be added and the hydrogenation continued if it has been determined (e.g. via in-process analysis) that the reaction has not gone substantially to completion and starting material remains.

There is no particular limitation on the pressure at which the hydrogenation is carried out. In this regard, the hydrogenation may conveniently be carried out with an initial hydrogen pressure in the range of up to about 7 bar (about 100 psi) e.g. about 5±1 bar.

The reaction temperature may be suitably in the range from about 15 to about 75° C., such as in the range from about 20 to about 60° C., for example, about 25 to about 50° C. In one embodiment, the reaction temperature may be about 30° C.

The reaction mixture may then be stirred in the presence of hydrogen gas until hydrogen uptake is no longer apparent. The hydrogenation reaction is carried out for a period of time until it is determined that the reaction is substantially complete. Completion of the reaction may be determined by in-process analysis or by identifying that there is no longer an uptake of hydrogen gas. Typically the hydrogenation is complete within about 24 hours, and in some embodiments, within about 90 minutes.

On completion of the reaction, the reaction vessel may be cooled to ambient temperature and purged with one or more nitrogen/vacuum cycles (e.g. one, two, three, four or five cycles) to remove excess hydrogen gas. The hydrogenation catalyst may be removed by any appropriate method, such as filtration (e.g. using a pad of Celite), washed one or more times with alcohol solvent (e.g. one, two, three or more times) and the filtrate further treated as desired. A proportion of the solvent may be evaporated if desired prior to recovery of the compound of formula (4a) or (4b).

Howsoever the compound (4a) or (4b) is recovered, the separated compounds may be washed and then dried. Drying may be performed using known methods, for example at temperatures in the range 10-60° C. and preferably 20-40° C. under 1-30 mbar vacuum for 1 hour to 5 days. If desired the compound (4a) or (4b) may be recrystallised, although in certain embodiments this is generally not required and the compounds (4a) and (4b), or salts thereof, may be used to form compounds (1a) and (1 b), or salts thereof, without further purification.

In this embodiment, in the compounds (6a) and (6b), R₁ may be as generally described above or may be OH. In one embodiment, R₁ is OH i.e. the —(R₃)C═N(OH) group is an oxime. In this instance, the compounds (6a) and (6b) have the following structure:

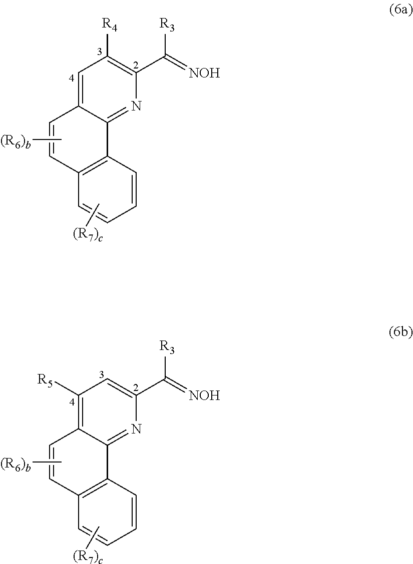

In this embodiment, when the —(R₃)C═N(OH)— group is hydrogenated, the OH is replaced by a H during the reaction. The compound (1a) or (1 b), therefore, may be prepared directly from a compound (6a) or (6b) as the compound (1a) or (1b) comprises a primary amine i.e. an NH₂ group.

Alternatively, when R₁ is OH for the compounds (6a) and (6b), the oxime group —(R₃)C═N(OH) may be reduced to the primary amine using a reducing agent selected from the group consisting of lithium aluminium hydride (LiAlH₄), LiAlH(OMe)₃, LiAlH(OEt)₃, AlH₃, BH₃.THF (borane tetrahydrofuran complex) solution, BH₃.DMS (borane dimethyl sulfide complex) solution, sodium borohydride (NaBH₄) and B₂H₆. In one embodiment, the reducing agent may be LiAlH₄. In another embodiment, the reducing agent may be NaBH₄.

In another embodiment, when R₁ is OH for the compounds (6a) and (6b), the oxime group —(R₃)C═N(OH) may be reduced to the primary amine using a reducing agent which is zinc and acetic acid.

In another embodiment, the reduction may be a transfer hydrogenation reaction. The transfer hydrogenation reaction may comprise reacting a compound (6a) or (6b) with a hydrogen donor in the presence of a transfer hydrogenation catalyst. The hydrogen donor may be selected from formic acid, a formic acid alkali salt (for example, sodium formate) and an alcohol, such as an alcohol having a hydrogen atom at a carbon that is a to the carbon atom to which the alcohol group is attached. An example of a suitable alcohol includes but is not limited to iso-propanol. In this embodiment, hydrogen is formally added across the —(R₃)C═N(R₁)— double bond, however, gaseous hydrogen (H₂) is not the source.

The transfer hydrogenation catalyst may be catalysts of the type [(sulphonylated diamine) RuCl (arene)] or heterogeneous PGM catalysts as described above.

In this embodiment, R₁ is not OH and is as generally described above.

When R₁ is not H or OH, the compound (6a) or (6b) may be reduced with an achiral catalyst to form a racemate. Compounds (4a) and (4b) can then be obtained in enantiomerically pure form by resolution of the racemic mixture as generally described above. Suitable acid resolving agents are also as generally described above.

Alternatively, when R₁ is not H or OH, the compound (6a) or (6b) may be asymmetrically reduced with a chiral catalyst to produce an enantiomerically enriched compound (4a) or (4b). Each enantiomer is within the scope of the present invention.

The compounds of formula (6a) or (6b) may form a salt with a suitable acid. The compounds (6a) and (6b) may be reacted as the free base with a suitable acid to form the salt. Alternatively, the acid may be present in situ during the preparation of compounds (6a) and (6b). For example, the compounds (7a) and (7b), described below, may be reacted as acid addition salts of compounds (7a) and (7b) forming the acid addition salts of compounds (6a) and (6b). The extra addition of acid to the reaction mixture comprising compounds (7a) and (7b), therefore, may not be necessary in order to prepare salts of compounds (6a) and (6b). Suitable acids are as generally described above.

In one embodiment, the acid may be a hydrohalide acid, such as hydrochloric acid, hydrobromic acid or hydroiodic acid. The salts of compounds (6a) and (6b) may accordingly be hydrochloride salts, hydrobromide salts or hydroiodide salts. In one embodiment, the salt is a hydrochloride salt.

### Preparation of Compounds of Formulae (6a) and (6b)

The compound (6a) or (6b), or salts thereof, may be prepared by the reaction of a compound of formula (7a) or (7b). In this respect, a compound (7a) reacts to form a compound (6a), or salt thereof, and a compound (7b) reacts to form a compound (6b), or salt thereof.

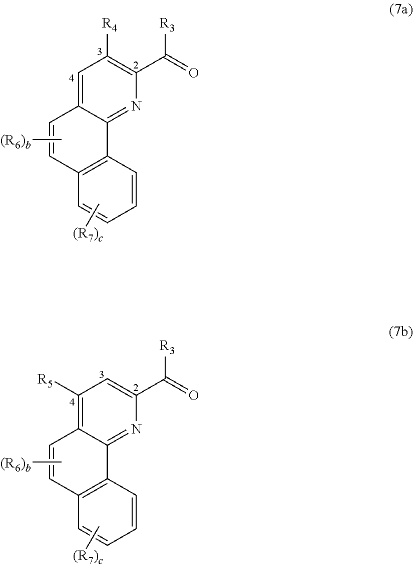

R₃, R₄, R₅, R₆, R₇, b and c are as generally described above.

Compounds (7a) and (7b) may be reacted with a compound of formula (8), or salt thereof, in an alcohol solvent to form compound (6a) or (6b).

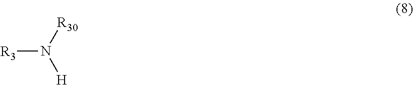

wherein,
R₃ is as defined above; and
R₃₀ is selected from the group consisting of H and OH.

The compound (8) reacts with the carbonyl group of compounds (7a) and (7b) to form the iminyl group of compounds (6a) and (6b). In one embodiment, R₃₀ is H i.e. the compound (8) is a primary amine. In another embodiment, R₃₀ is OH i.e. the compound (8) is a hydroxylamine.

Salts of compounds (8) may be used in this reaction. The salts of compounds (1a) or (1b) may be hydrochloride salts, hydrobromide salts or hydroiodide salts. In one embodiment, the salt is a hydrochloride salt. Salts of compounds (6a) and (6b) may be precipitated from the reaction mixture when salts of compounds (8) are utilised as a reactant, thus facilitating the isolation of the compounds (6a) and (6b) and, if desired, subsequent purification.

When compound (8) is a hydroxylamine (i.e. when R₃₀ is OH) and the hydroxylamine is reacted as the hydrochloride salt, the inventors have noted that the oxime hydrochlorides (6a) and (6b) may precipitate from the reaction mixture as stable solids.

The compound (8), or salt thereof, may be present in stoichiometric or greater quantities to the compound (7a) or (7b). The molar ratio of the compound (7a) or (7b) to compound (8), or salt thereof, may be in the range of about 1 to about 5, such as about 1 to about 3, for example, about 1 to about 2. In one embodiment, the molar ratio of the compound (7a) or (7b) to compound (8), or salt thereof, is about 1 to about 1. In another embodiment, the molar ratio of the compound (7a) or (7b) to compound (8), or salt thereof, is about 1 to about 1.8.

When the free base of compound (7a) or (7b) is reacted, stoichiometric or slight excess of base may be suitable, for example, about 1:about 1.1 to about 1:about 1.5 molar ratio of compound (1a) or (1 b) to base.

The reaction comprises an alcohol solvent. The alcohol may be selected from the group consisting of methanol, ethanol, isopropanol and mixtures thereof. In one embodiment, the solvent is ethanol. The concentration of compound (7a) or (7b) in the alcohol solvent may be about 0.001 mol/L to about 1.0 mol/L, such as about 0.01 to about 0.75 mol/L, for example, about 0.1 mol/L to about 0.5 mol/L. In one embodiment, the concentration of compound (7a) or (7b) in the alcohol solvent is about 0.2 to about 0.4 mol/L, for example, about 0.28 mol/L or about 0.37 mol/L.

The compound (7a) or (7b), the solvent and the compound (8) may be added in any suitable order. In one embodiment, however, the compound (7a) or (7b) is suspended in the alcohol solvent in a reaction vessel, optionally heated to temperature, and then the compound (8) is added. The compound (8) may be added in one portion or portionwise. In one embodiment, the compound (8) is added in one portion. When compound (8) is a hydroxylamine hydrochloride (i.e. when R₃₀ is —OH), the reaction mixture may form a solution on addition of the hydroxylamine.

The reaction temperature may be suitably in the range from about 15 to about 75° C., such as in the range from about 20 to about 60° C., for example, about 25 to about 50° C. In one embodiment, the reaction temperature may be about 40° C.

The reaction is carried out for a period of time until it is determined that the reaction is substantially complete. Completion of the reaction may be determined by in-process analysis. Typically the reaction is complete within about 24 hours, and in some embodiments, within about 90 minutes.

On completion of the reaction, the reaction mixture may be cooled (e.g. to 0° C. using an ice-bath). When a free base of compound (8) has been used, the free base of the compounds (6a) and (6b) may be isolated as the product by evaporating a proportion of the solvent. Alternatively, salts of compounds (6a) and (6b) may be isolated by treating the reaction mixture comprising the free bases of the compounds (6a) and (6b) with a suitable acid. Suitable acids are as generally described above. In one embodiment, the acid may be a hydrohalide acid, such as hydrochloric acid, hydrobromic acid or hydroiodic acid. The salts of compounds (6a) and (6b) may accordingly be hydrochloride salts, hydrobromide salts or hydroiodide salts. In one embodiment, the salt is a hydrochloride salt. In yet another embodiment, salts of compounds (6a) and (6b) may be obtained on utilising a salt of compound (8). In this instance, on completion of the reaction and on cooling the reaction vessel additional product may precipitate from the reaction mixture. The solid may be filtered and washed one or more times with alcohol solvent (e.g. one, two, three or more times).

Howsoever the compound (6a) or (6b), or salt thereof, is recovered, the compounds may be dried. Drying may be performed using known methods, for example at temperatures in the range 10-60° C. and preferably 20-40° C. under 1-30 mbar vacuum for 1 hour to 5 days. Typically, the compounds (6a) and (6b), or salts thereof, may be used to form the compounds (4a) and (4b) without further purification.

### Preparation of the Compounds of Formulae (7a) and (7b)

The compounds of formula (7a) or (7b) may be prepared in a process comprising the steps of:
(a) reacting a compound of formula (9a) or (9b) with a lithiating agent in an ethereal solvent to form the lithiated compound (10a) or (10b); and

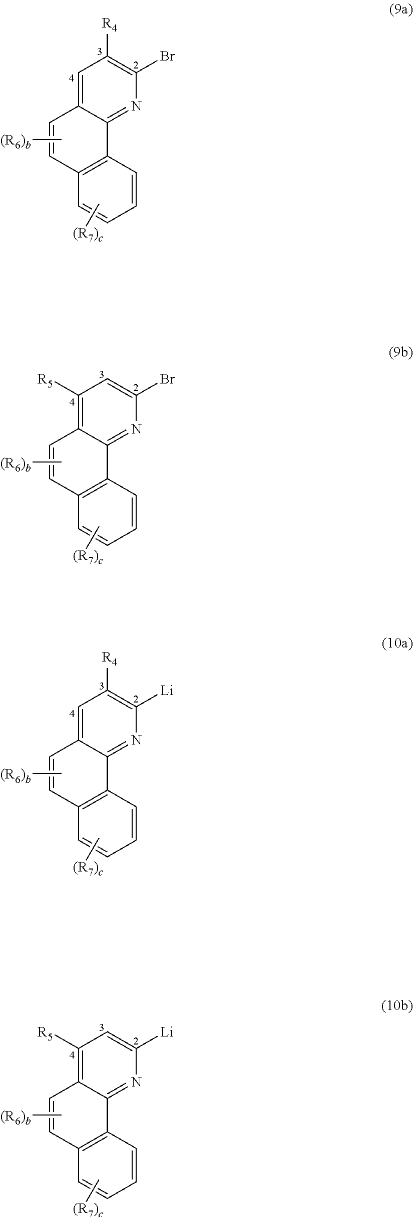

(b) reacting the lithiated compound (10a) or (10b) with a compound of formula (11) to form the compound of formula (7a) or (7b).

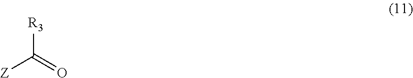

wherein:
R₃, R₄, R₅, R₆, R₇, b and c are as generally described above; and

### Z is —N(alkyl)₂ or -Hal.

A compound (9a) reacts via compound (10a) to form a compound (7a) and a compound (9b) reacts via a compound (10b) to form a compound (7b).

The lithiating agent may be an alkyl lithium reagent, such as n-BuLi or sec-BuLi. The alkyl lithium reagent may be conveniently purchased as a solution in a solvent, such as hexane. Stoichiometric or slight excess of lithiating agent may be used. For example, the molar ratio of compound (9a) or (9b) to lithiating agent may be about 1 to about 1 or about 1.1 to about 1 to about 1.5, such as about 1 to about 1.25.

The ethereal solvent may be an alkyl ether. Preferably, the alkyl ether is anhydrous. In one embodiment, the alkyl ether is a cyclic alkyl ether and more preferably tetrahydrofuran (THF). In another embodiment, the alkyl ether is diethyl ether or methyl tert-butyl ether (MTBE). With regard to THF and MTBE, the use of alkyl ethers such as these have higher flashpoint temperatures and, as such, may provide improved safety in handling. The concentration of compound (9a) or (9b) in the ethereal solvent may be about 0.001 mol/L to about 1.0 mol/L, such as about 0.01 to about 0.9 mol/L, for example, about 0.1 mol/L to about 0.85 mol/L. In one embodiment, the concentration of compound (9a) or (9b) in the ethereal solvent is about 0.25 to about 0.8 mol/L, for example, about 0.72 mol/L or about 0.33 mol/L.

The solution of the compound (9a) or (9b) may be cooled to e.g. about −78° C. before the lithiating agent is added. In this respect, the reaction temperature at which the lithiating reaction may occur can be suitably in the range from about −78 to about −20° C., such as in the range from about −78 to about −50° C. In one embodiment, the reaction temperature may be about −78° C. An isopropanol/dry ice bath may be used to cool the reaction mixture to about −78° C.

The compound (9a) or (9b), the ethereal solvent and the lithiating agent may be added in any suitable order. In one embodiment, the compound (9a) or (9b) is dissolved in the ethereal solvent in a reaction vessel, cooled, before adding the lithiating agent. The lithiating agent may be added in one portion or portionwise (e.g. dropwise) over a period of time. In one embodiment, the lithiating agent is added portionwise. The lithiating agent may be added using a syringe or a dropping funnel. If desired, the syringe or dropping funnel may be washed with a portion of ethereal solvent and the wash added to the reaction mixture.

The reaction mixture of step (a) is stirred for a period of time of up to about 3 hours when reacting compounds (9a) and (9b) with the lithiating agent on a scale of about 22 g or less. For larger reactions, however, the lithiating step may require a longer reaction time.

The compound of formula (11) is added to the reaction mixture comprising the compound (10a) or (10b) to form the compound (7a) or (7b). Stoichiometric or excess of compound (11) may be used. For example, the molar ratio of compound (9a) or (9b) to compound (11) may be about 1 to about 1 or about 1 to about 1.1 to about 1 to about 1.5, such as about 1 to about 1.25.

The compound (11) may be selected from the group consisting of N,N-dimethylformamide (DMF), N,N-dimethylacetamide (DMA), N,N-dimethylpropionamide, N,N-dimethylbutionamide and N,N-dimethylbenzamide. DMF provides a compound (7a) or (7b) where R₃ is —H, DMA provides a compound (7a) or (7b) where R₃ is -Me, N,N-dimethylpropionamide provides a compound (7a) or (7b) where R₃ is -Et, N,N-dimethylbutionamide provides a compound (7a) or (7b) where R₃ is —Bu and N,N-dimethylbenzamide provides a compound (7a) or (7b) where R₃ is -Ph.

Step (b) may be carried out at one or more temperatures in the range of about −78 to about 30° C. In one embodiment, the compound (11) is reacted with the compound (10a) or (10b) at a temperature lower than −65° C. and the reaction mixture allowed to warm slowly to room temperature.

Step (b) is carried out for a period of time until it is determined that the reaction is substantially complete. Completion of the reaction may be determined by in-process analysis. Typically the reaction is complete within about 24 hours, and in some embodiments, within about 16 hours.

Steps (a) and (b) are typically conducted under an inert atmosphere, such as nitrogen or argon.

On completion of the reaction, an alcohol (e.g. methanol) and an organic acid (e.g. acetic acid) may be added to quench the reaction mixture, followed by the addition of water and an aprotic solvent (such as dichloromethane). The organic phase may be separated from the aqueous phase and the organic phase washed one or more times with water (e.g. one, two, three or more times), one or more times with brine (e.g. one, two, three or more times), dried (e.g. using magnesium sulfate) and concentrated in vacuo to give the compound (7a) or (7b) as an oil or solid. Typically, the compounds (7a) and (7b) may be used to form the compounds (6a) and (6b) without further purification.

### Preparation of the Compounds of Formulae (9a) and (9b)

The compound of formula (9a) or (9b) may be prepared in a process comprising the reaction of a compound of formula (12a) or (12b) with a halogenating agent in a solvent.

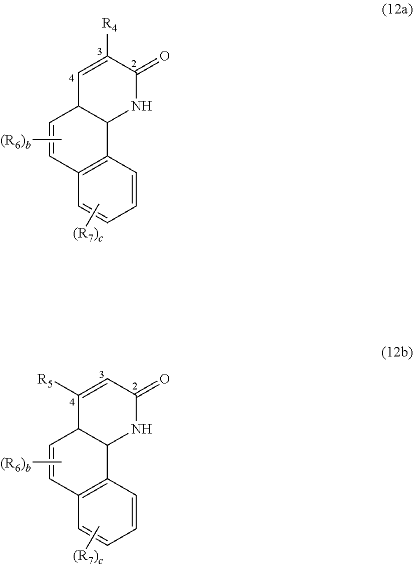

wherein:
R₄, R₅, R₆, R₇, b and c are as generally described above.

The compound (12a) reacts to form the compound (9a) and the compound (12b) reacts to form the compound (9b).

The halogenating agent may be a brominating agent or a chlorinating agent. The halogenating agent may be selected from the group consisting of phosphoryl bromide (POBr₃) and phosphoryl chloride (POCl₃). In one embodiment, the halogenating agent is POBr₃. In another embodiment, the halogenating agent is POCl₃.

Any suitable solvent may be used, for example, an aromatic hydrocarbon, such as benzene, toluene or xylene or amide solvent, such as dimethylformamide or dimethacetamide. In one embodiment, the aromatic solvent is toluene. In another embodiment, the amide solvent is dimethylformamide. In one embodiment, the solvent is anhydrous. The concentration of compound (12a) or (12b) in the solvent may be about 0.001 mol/L to about 2.0 mol/L, such as about 0.01 to about 1.75 mol/L, for example, about 0.05 mol/L to about 1.5 mol/L. In one embodiment, the concentration of compound (12a) or (12b) in the solvent is about 0.5 to about 2.0 mol/L, for example, about 0.7 to about 1.0, such as about 0.74 mol/L or about 0.75 mol/L or about 0.969 mol/L. In one embodiment, the concentration of compound (12a) or (12b) in the solvent is about 0.01 to about 0.5 mol/L, for example, about 0.05 to about 0.1 mol/L, such as about 0.06 mol/L.

If desired, the compound (12a) or (12b) may be azeotropically dried before it is reacted with the halogenating agent.

The compound (12a) or (12b), the solvent and the halogenating agent may be added in any suitable order. In one embodiment, however, the compound (12a) or (12b) and halogenating agent are combined with the solvent in a reaction vessel. In another embodiment, the compound (12) or (12b) is charged to a reaction vessel with the solvent, followed by the addition of the halogenating agent.

The reaction mixture may be heated to a temperature in the range from about 50 to about 200° C., such as in the range from about 60 to about 175° C., for example, about 75 to about 160° C. In one embodiment, the reaction may be heated to the reflux temperature of the solvent. Accordingly, when the solvent is benzene, the reaction temperature may be the boiling point of benzene i.e about 80° C. When the solvent is toluene, the reaction temperature may be the boiling point of toluene i.e. about 111° C. When the solvent is xylene, the reaction temperature may be in the boiling point of xylene i.e. in the range of about 138 to about 144° C. When the solvent is dimethylformamide, the reaction temperature may be the boiling point of DMF i.e. about 153° C.

The reaction may be conducted under an inert atmosphere, such as argon or nitrogen.

The reaction is carried out for a period of time until it is determined that the reaction is substantially complete. Completion of the reaction may be determined by in-process analysis. Typically the reaction is complete within about 24 hours, and in some embodiments, within about 16 hours. Hydrogen halide (e.g. HBr or HCl) may be formed during the course of the reaction which may be released through the use of a bubbler.

On completion of the reaction, the reaction mixture may be suspended in ice/water, stirred for a period of time (e.g. about 2 hours), filtered and dried in vacuum. Drying may be performed using known methods, for example at temperatures in the range 10-60° C. and preferably 20-40° C. under 1-30 mbar vacuum for 1 hour to 5 days.

Alternatively, the reaction mixture may be cooled (e.g. to room temperature). Water may be added to the reaction mixture and optionally an inorganic base. Examples of suitable inorganic bases include but are not limited to hydroxides and alkoxides. Suitable hydroxides include alkali metal hydroxides (e.g. lithium hydroxide, sodium hydroxide or potassium hydroxide) or tetraalkylammonium hydroxides (e.g. tetrabutylammonium hydroxide). In one embodiment, the inorganic base is a hydroxide which is sodium hydroxide. Sodium hydroxide may be added to the reaction mixture until the pH is about 10-14. Suitable alkoxides include alkali metal alkoxides (e.g. lithium alkoxide, sodium alkoxide or potassium alkoxide, such as lithium methoxide, sodium methoxide or potassium methoixde) or tetraalkylammonium alkoxides (e.g. tetrabutylammonium hydroxide).

The aqueous and organic phases may be separated and the aqueous phase washed one or more times with solvent (for example, one, two or three times with an aromatic solvent as described above). The organic phases may be combined and washed one or more times with brine (e.g. one, two, three or more times), dried (e.g. using magnesium sulfate) and concentrated in vacuo to give the compound (9a) or (9b). The compound (9a) or (9b) may be dissolved in a polar aprotic solvent (such as dichloromethane), optionally passed through a pad of silica gel, and the solvent removed in vacuo to provide a pure product.

Alternatively, the combined organic phases may be dried and concentrated in vacuo. The product may be taken up in a ketone solvent (e.g. acetone) and the solution heated to reflux, before being filtered hot. The ketone solvent may then be partially evaporated to produce a slurry, which may be filtered and dried.

Typically, the compounds (9a) and (9b) may be used to form the compounds (7a) and (7b) without further purification.

### Preparation of the Compounds of Formula (12a) and (12b)

The compound of formula (12a) or (12b) may be prepared in a process comprising the step of reacting a compound of formula (13a) or (13b) with an acid.

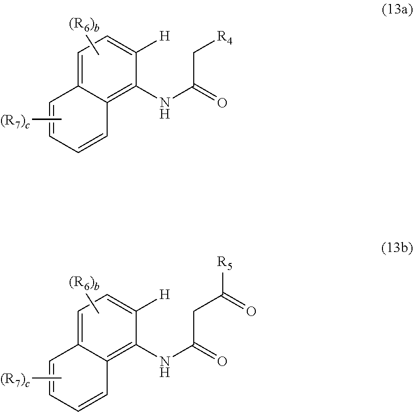

wherein:
R₄, R₅, R₆, R₇, b and c are as generally described above.

The compound (13a) reacts to form the compound (12a) and the compound (13b) reacts to form the compound (12b).

Any suitable acid may be used which is capable of cyclising the compound (13a) or (13b) to form the compound (12a) or (12b). The acid may be mineral acid, such as sulphuric acid or hydrochloric acid. In one embodiment, the acid may be concentrated acid (e.g. 98% sulphuric acid). In another embodiment, the acid may be an aqueous solution of acid. Any suitable w/w ratio of water:acid may be used. For example, the w/w/ratio of water:acid may be from about 10:about 0.01 to about 0.01: about 10, such as about 5:about 1 to about 1:about 5, e.g. about 1:about 3. The quantities of water and/or acid are not particularly limiting provided there is enough water and/or acid to cyclise the compound (13a) or (13b) into the compound (12a) or (12b).

The w/w ratio of compound of formula (13a) or (13b): acid may be in the range from about 10:about 0.01 to about 0.01:about 10, such as about 5:about 1 to about 1:about 5, e.g. about 1:about 3.

The acid may be heated to a temperature in the range of about 50 to about 95° C., such as about 50 to about 85° C., for example about 60 to about 80° C. e.g. about 75° C. before it is reacted with the compound (13a) or (13b). The compound (13a) or (13b) and the acid may be added in any suitable order. In one embodiment, however, the acid is charged to a reaction vessel and the compound (13a) or (13b) is added to the acid. The compound (13a) or (13b) may be added in one portion or portionwise over a period of time (e.g. 30 minutes). In another embodiment, the compound (13a) or (13b) is charged to a reaction vessel and the acid is added to the compound (13a) or (13b). The acid may be added in one portion or portionwise over a period of time.

The reaction mixture may be heated to a temperature in the range from about 50 to about 100° C., such as in the range from about 60 to about 100° C., for example, about 75 to about 100° C. The reaction mixture is typically stirred during the course of the reaction and if any lumps of solid are produced, these may be broken up as appropriate (e.g. using a Teflon rod).

The reaction is carried out for a period of time until it is determined that the reaction is substantially complete. Completion of the reaction may be determined by in-process analysis. Typically the reaction is complete within about 24 hours, and in some embodiments, within about 5 hours.

On completion of the reaction, the reaction mixture may be cooled (e.g. to room temperature). The reaction mixture may be diluted with water e.g. by adding the reaction mixture to water or adding water to the reaction mixture to afford a precipitate. The precipitate may be filtered and optionally washed one or more times with water (e.g. one, two, three or more times) and dried. In one embodiment, the precipitate may then crystallised from ethanol and the solid obtained stripped with an aromatic hydrocarbon solvent, such as toluene, one or more times (e.g. one, two, three or more times) to remove residual water. In another embodiment, the precipitate may be washed with a ketone solvent, such as acetone, one or more times (e.g. one, two, three or more times) and the solid dried.

Howsoever the compound (12a) or (12b) is recovered, the compounds may be dried. Drying may be performed using known methods, for example at temperatures in the range 10-60° C. and preferably 20-40° C. under 1-30 mbar vacuum for 1 hour to 5 days. Typically, the compounds (12a) and (12b) may be used to form the compounds (9a) and (9b) without further purification.

### Preparation of the Compound of Formula (13a)

The compound of formula (13a) may be prepared in a process comprising the step of reacting a naphthylamine of formula (14), or salt thereof, with a compound of formula (15):

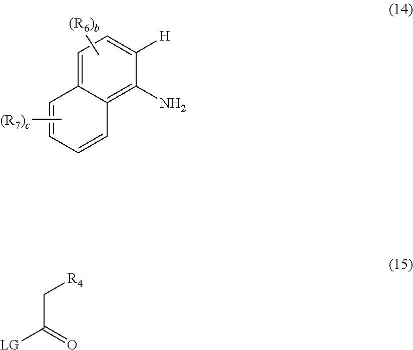

wherein:
R₄, R₆, R₇, b and c are as generally described above; and
LG is a leaving group.

The naphthylamine of formula (14) may be a free base or salt thereof. In one embodiment, the salt of compounds (14) may be a hydrochloride salt, hydrobromide salt or hydroiodide salt.

LG is a leaving group which may be selected from the group consisting of a halide, —O-alkyl and a sulfonate ester. In one embodiment, the leaving group is a halide, such as —Cl, —Br or —I. In another embodiment, the leaving group is an —O-alkyl, such as —O-Et or -Me.

In one embodiment, the compound of formula (15) is propionyl chloride.

The reaction may further comprise a base. Any suitable base may be used which is capable of deprotonating the NH₂ group of the compound (14) but does not otherwise adversely affect the reaction. Suitable bases include but are not limited to inorganic bases, such as sodium acetate, and organic bases, such as lutidine or triethylamine.

The compound (15) may be present in stoichiometric or greater quantities to the compound (14), or salt thereof. When the free base of compound (15) is reacted, stoichiometric or slight excess of base may be suitable, for example, about 1:1.1 to 1:1.5 molar ratio of compound (15) to base. When salts of compound (15) are utilised, however, excess base is generally required in order to form the free base of the compound (15) from the salt of compound (15), and deprotonate the amino group. In this respect, the molar ratio of the salts of compound (15) to base may be about 1:5 to about 1:20, such as about 1:7.5 to about 1:15, such as about 1:10.

The reaction may further comprise a solvent. Any suitable solvent may be used, for example, chlorinated solvents, such as dichloromethane (DCM), aromatic hydrocarbons, such as benzene, toluene or xylene, or ethereal solvents, for example alkyl ethers, such as THF or MTBE. In one embodiment, the solvent is xylene. The concentration of compound (14) in the solvent may be about 0.001 mol/L to about 10.0 mol/L, such as about 0.01 to about 7.5 mol/L, for example, about 0.05 mol/L to about 5.0 mol/L. In one embodiment, the concentration of compound (14) in the solvent is about 0.78 mol/L.

The reaction may be conducted under an inert atmosphere, such as argon or nitrogen.

The compound (14), the compound (15), the base (if any) and the solvent (if any) may be added in any suitable order. In one embodiment of the invention, however, the compound (14) and the solvent (if any) are charged to a reaction vessel, the base (if any) and compound (15) are added.

While the compound (15) is added to the reaction mixture, the temperature range of the reaction may generally be maintained at one or more temperatures between about 10° C. to about 35° C. In one embodiment, the reaction mixture is maintained at a temperature of less than about 5° C., such as about 0° C. In order to keep the temperature of the reaction mixture within these ranges, the compound of formula (15) may be added slowly over a period of time.

The reaction may be continued for a period of from about 30 minutes to about 72 hours, such as about 30 minutes to about 24 hours. During this time, the reaction temperature may be varied one or more times between about 10° C. and about 25° C. On completion of the reaction, the precipitate may be filtered off and the filtrate extracted with one or more times (e.g. one, two, three or more times) with e.g. DCM/10% HCl. The organic layer may be separated from the aqueous layer and the organic layers combined, dried (e.g. using magnesium sulfate) and concentrated in vacuo. Drying may be performed using known methods, for example at temperatures in the range 10-60° C. and preferably 20-40° C. under 1-30 mbar vacuum for 1 hour to 5 days. Typically, the compound (13a) may be used to form the compound (12a) without further purification.

### Preparation of the Compound of Formula (13b)

The compound of formula (13b) may be prepared by reacting a compound of formula (14) with a compound of formula (16) or a compound of formula (17).

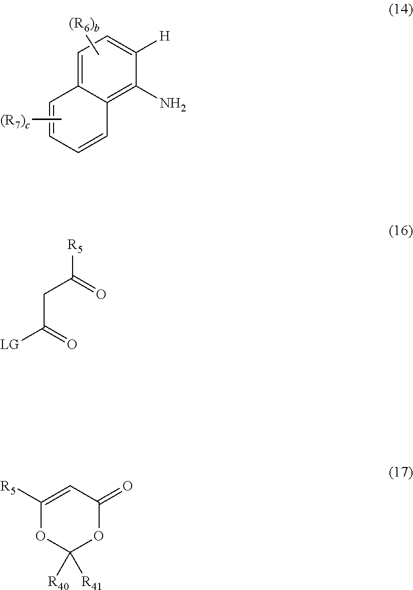

wherein:
R₅, R₆, R₇, b and c are as generally defined above;
R₄₀ and R₄₁ are independently selected from the group consisting of unsubstituted alkyl and substituted alkyl, or R₄₀ and R₄₁ are interconnected to form a ring with the carbon to which they are attached; and
LG is a leaving group.

In one embodiment, R₄₀ and R₄₁ are methyl groups.

When R₄₀ and R₄₁ are interconnected to form a ring with the carbon atom to which they are attached, the groups may form substituted or unsubstituted chiral or achiral bridges which are derived, for example, from the skeletons —(CH₂)ₙ— (n=2, 3 or 4), —CH(CH₃)CH(CH₃)—, —CH(CH₃)CH₂CH(CH₃)—, —CMe₂-, —CHMe-, no limitation being implied by this listing.

LG is a leaving group which may be selected from the group consisting of a halide, —O-alkyl and a sulfonate ester. In one embodiment, the leaving group is a halide, such as —Cl, —Br or —I. In another embodiment, the leaving group is an —O-alkyl, such as —O-Et or —O-Me.

The reaction may further comprise a base. Any suitable base may be used which is capable of deprotonating the —NH₂ group of the compound (14) but does not otherwise adversely affect the reaction. Suitable bases include but are not limited to inorganic bases, such as sodium acetate, and organic bases, such as lutidine or triethylamine.

The compound (14) may be present in stoichiometric or greater quantities to the compound (14), or salt thereof. When the free base of compound (14) is reacted, stoichiometric or slight excess of base may be suitable, for example, about 1:1.1 to 1:1.5 molar ratio of compound (14) to base. When salts of compound (14) are utilised, however, excess base is generally required in order to form the free base of the compound (14) from the salt of compound (14), and deprotonate the amino group. In this respect, the molar ratio of the salts of compound (14) to base may be about 1:5 to about 1:20, such as about 1:7.5 to about 1:15, such as about 1:10.

The reaction may further comprise a solvent. Any suitable solvent may be used, for example, chlorinated solvents, such as dichloromethane (DCM), aromatic hydrocarbons, such as benzene, toluene or xylene, or ethereal solvents, for example alkyl ethers, such as THF or MTBE. In one embodiment, the solvent is xylene. The concentration of compound (14) in the solvent may be about 0.001 mol/L to about 10.0 mol/L, such as about 0.01 to about 7.5 mol/L, for example, about 0.05 mol/L to about 5.0 mol/L. In one embodiment, the concentration of compound (14) in the solvent is about 0.78 mol/L. In another embodiment, the concentration of compound (14) in the solvent is about 4.11 mol/L.

The napthylamine of formula (14), LG, the base (if any), the solvent (if any) are as generally described above.

The compound (16) or (17) may be present in stoichiometric or greater quantities to the compound (14), or salt thereof. When the free base of compound (14) is reacted, stoichiometric or slight excess of compound (16) or (17) may be suitable, for example, about 1:1.1 to 1:1.5 molar ratio of compound (14) to compound (16) or (17). When salts of compound (14) are utilised, however, excess base is generally required in order to form the free base of the compound (14) from the salt of compound (14), and deprotonate the amino group. In this respect, the molar ratio of the salts of compound (14) to base may be about 1:5 to about 1:20, such as about 1:7.5 to about 1:15, such as about 1:10.

The reaction may be conducted under an inert atmosphere, such as argon or nitrogen.

The compound (14), the compound (16) or (17), the base (if any) and the solvent (if any) may be added in any suitable order. In one embodiment of the invention, however, the compound (14) and the solvent (if any) are charged to a reaction vessel, the base (if any) and compound (16) or (17) are added.

While the compound (16) or (17) is added to the reaction mixture, the temperature range of the reaction may generally be maintained at one or more temperatures between about 50° C. to about 200° C. The temperature selected is such that the desired amide is formed instead of an imine. Without wishing to be bound by theory, it is believed that higher temperatures (e.g. by refluxing the reaction mixture in xylene) favour the formation of the desired amide, whereas lower temperatures favour the formation of an imine. In one embodiment, the reaction mixture is maintained at a temperature of less than about 175° C., such as about 160-165° C. In another embodiment, the reaction is maintained at the reflux temperature of THF i.e. at about 66° C.

The reaction may be continued for a period of from about 30 minutes to about 72 hours, such as about 30 minutes to about 24 hours. On completion of the reaction, the reaction mixture may be concentrated in vacuo until the product solidifies in the reaction flask. The precipitate may be collected using an alkane solvent (such as hexane or heptane) to do so and optionally washed one or more times with further alkane solvent (such as hexane or heptane). Alternatively, aqueous acid (e.g. aqueous HCl acid) may be added to the reaction mixture with vigorous stirring for a period of time before filtering the precipitate. The precipitate may then be washed one or more times with water and dried in a desiccator.

The precipitate may be dried using known methods, for example at temperatures in the range 10-60° C. and preferably 20-40° C. under 1-30 mbar vacuum for 1 hour to 5 days.

Alternatively, on completion of the reaction, the reaction mixture may be diluted with an ester solvent (such as ethyl acetate), washed one or more times (e.g. one, two, three or more times) with water, washed one or more times (e.g. one, two, three or more times) with brine and dried (e.g. over sodium sulfate). The product may be obtained by removal of the organic solvents, such as by increasing the temperature or reducing the pressure using distillation or stripping methods well known in the art.

The compound of formula (13b) may be used to form the compound (12b) without further purification.

### Preparation of Compounds of Formulae (1a) and (1b)

In addition to the process described above, the compounds of formulae (1a) and (1b), or salts thereof, (illustrated below) may be prepared by reducing a compound of formula (20a) or (20b), or salts thereof. A compound (20a) is reduced to the compound (1a) and the compound (20b) is reduced to the compound (1b).

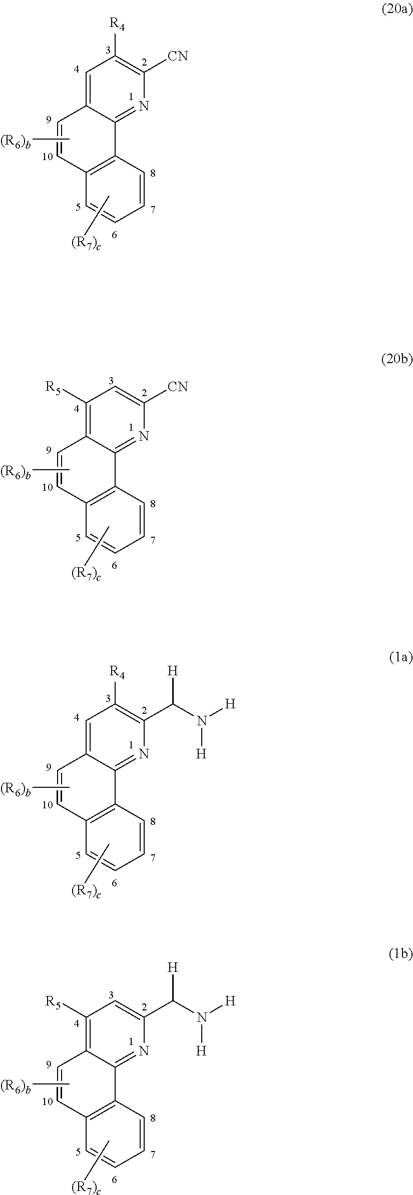

As the process comprises the reduction of a cyano (—CN) group, R₁, R₂ and R₃ in the compounds of formulae (1a) and (1 b) are all —H.

R₄, R₅, R₆, R₇, b and c are as generally described above.

In one embodiment, the reduction may be a hydrogenation reaction. The hydrogenation reaction may comprise reacting the compound (20a) or (20b) with gaseous hydrogen in the presence of a hydrogenation catalyst in a suitable solvent. The hydrogenation catalyst may be a heterogeneous or homogeneous catalyst, preferably a heterogeneous catalyst. The catalyst (whether heterogeneous or homogeneous) should be selected such that the catalyst preferentially reduces the cyano (—CN) group rather than reducing another group present in the compound (20a) or (20b). In one embodiment, the heterogeneous catalyst is a heterogeneous platinum group metal (PGM) catalyst, for example, a heterogeneous palladium or platinum catalyst. In one embodiment, the heterogeneous catalyst is a heterogeneous palladium catalyst. Examples of palladium catalysts include but are not limited to colloidal palladium, palladium sponge, palladium plate or palladium wire. Examples of platinum catalysts include but are not limited to colloidal platinum, platinum sponge, platinum plate or platinum wire.

The heterogeneous PGM metal catalyst may be a PGM on a solid support. The support may be selected from the group consisting of carbon, alumina, calcium carbonate, barium carbonate, barium sulfate, titania, silica, zirconia, ceria and a combination thereof. When the support is alumina, the alumina may be in the form of alpha-Al₂O₃, beta-Al₂O₃, gamma-Al₂O₃, delta-Al₂O₃, theta-Al₂O₃ or a combination thereof. When the support is carbon, the carbon may be in the form of activated carbon (e.g. neutral, basic or acidic activated carbon), carbon black or graphite (e.g. natural or synthetic graphite). An example of a heterogeneous PGM catalyst is palladium on carbon. An example of another heterogeneous PGM catalyst is platinum on carbon.

The catalyst loading may be up to about 20 mole %. A greater catalyst loading may perform the desired reduction, however, increasing the quantity of the PGM may make the process uneconomical. In one embodiment, the catalyst loading may be up to 10 mole % and, in another embodiment, may be in the range of about 0.1-10.0 mole %.

The reaction mixture may further comprise an acid. Without wishing to be bound by theory, it is believed the acid helps the formation of the amine by avoiding dimerization side reactions. The acid may be any suitable acid, such as a hydrohalide acid e.g. hydrochloric acid, hydrobromic acid or hydroiodic acid. The acid may be added as a reagent to the hydrogenation reaction or the compounds (20a) and (20b) may be reacted as acid addition salts. The salts are as generally described above. Without wishing to be bound by theory, it is believed that the benzo-fused pyridinyl N atom needs to be protonated in order for the hydrogenation to proceed.

Any suitable solvent may be utilised e.g. polar solvents, such as an alcohol. The alcohol may be selected from the group consisting of methanol, ethanol, isopropanol and mixtures thereof. In one embodiment, the solvent is methanol.

The compound (20a) or (20b) may be placed in a pressure vessel together with the hydrogenation catalyst. The pressure vessel may then be assembled and purged with one or more nitrogen/vacuum cycles (e.g. one, two, three or four cycles). The alcohol solvent may then added via the injection port to form a solution of the compound (20a) or (20b), which may have concentration in the range of about 0.01 to about 1 molar, such as about 0.3 molar. If the hydrogenation catalyst is heterogeneous, the catalyst will not dissolve in the alcohol solvent. However, if the hydrogenation catalyst is homogeneous, it may dissolve in the alcohol solvent and form a solution with the compound (20a) or (20b).

Once the alcohol solvent has been added, the pressure vessel may be purged once again with one or more nitrogen/vacuum cycles (e.g. one, two, three, four or five cycles), followed by one or more hydrogen/vacuum cycles (e.g. one, two, three, four or five cycles). During purging the reaction mixture may be agitated (by either stirring or shaking) to encourage removal of dissolved oxygen. The pressure vessel may then be pressurised with hydrogen (e.g. to about 5 bar), stirred and heated to temperature (e.g. about 30° C.). Hydrogen gas uptake may begin after a period of time has elapsed. Once hydrogen uptake begins, the pressure vessel may optionally be depressurised with hydrogen

While it is typically sufficient for a single charge of hydrogenation catalyst to be added to the reaction mixture, a second or further charge may be added and the hydrogenation continued if it has been determined (e.g. via in-process analysis) that the reaction has not gone substantially to completion and starting material remains.

There is no particular limitation on the pressure at which the hydrogenation is carried out. In this regard, the hydrogenation may conveniently be carried out with an initial hydrogen pressure in the range of up to about 7 bar (about 100 psi) e.g. about 5±1 bar.

The reaction temperature may be suitably in the range from about 15 to about 75° C., such as in the range from about 20 to about 60° C., for example, about 25 to about 50° C. In one embodiment, the reaction temperature may be about 30° C.

The reaction mixture may then be stirred in the presence of hydrogen gas until hydrogen uptake is no longer apparent. The hydrogenation reaction is carried out for a period of time until it is determined that the reaction is substantially complete. Completion of the reaction may be determined by in-process analysis or by identifying that there is no longer an uptake of hydrogen gas. Typically the hydrogenation is complete within about 24 hours, and in some embodiments, within about 90 minutes.

On completion of the reaction, the reaction vessel may be cooled to ambient temperature and purged with one or more nitrogen/vacuum cycles (e.g. one, two, three, four or five cycles) to remove excess hydrogen gas. The hydrogenation catalyst may be removed by any appropriate method, such as filtration (e.g. using a pad of Celite), washed one or more times with alcohol solvent (e.g. one, two, three or more times) and the filtrate further treated as desired. A proportion of the solvent may be evaporated if desired prior to recovery of the compound of formula (1a) or (1 b).

Howsoever the compound (1a) or (1 b) is recovered, the separated compounds may be washed and then dried. Drying may be performed using known methods, for example at temperatures in the range 10-60° C. and preferably 20-40° C. under 1-30 mbar vacuum for 1 hour to 5 days. If desired the compound (1a) or (1 b) may be recrystallised, although in certain embodiments this is generally not required.

### Preparation of Compounds of Formulae (20a) and (20b)

The compounds of formulae (20a) and (20b) may be prepared by cyanating the compounds of formulae (9a) and (9b) (discussed above).

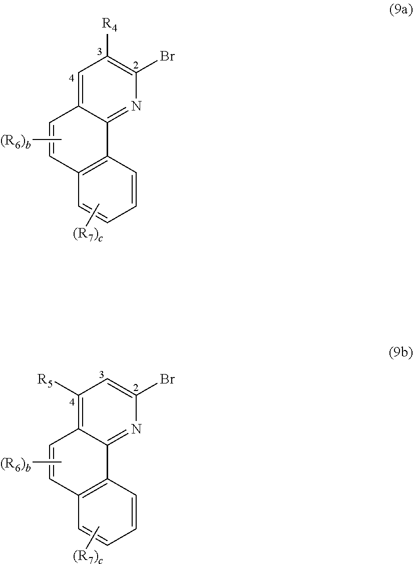

In this respect, the compound (9a) is cyanated to the compound (20a) and the compound (9b) is cyanated to the compound (20b).

R₄, R₅, R₆, R₇, b and c are as generally described above.

The process may comprise treating the compound of formula (20a) or (20b) with a cyanating reagent in solvent.

The cyanation reagent may be any suitable cyanation reagent, such as copper(I) cyanide, Zn(CN)₂ or K₄Fe(CN)₆ (potassium ferrocyanide).

The solvent may be any suitable solvent, such as polar aprotic solvents. Polar aprotic solvents may be selected from the group consisting of amides (such as N,N-dimethylformamide (DMF) or N,N-dimethylacetamide (DMA)) and N-(alkyl)-pyrrolidinones (such as N-methyl-2-pyrrolidinone). In one embodiment, the solvent is N-methyl-2-pyrrolidinone (NMP). In one embodiment, the solvent is anhydrous. The concentration of compound (9a) or (9b) in the solvent may be about 0.001 mol/L to about 2.0 mol/L, such as about 0.01 to about 1.75 mol/L, for example, about 0.05 mol/L to about 1.5 mol/L. In one embodiment, the concentration of compound (9a) or (9b) in the solvent is about 0.1 to about 1.0 mol/L, for example, about 0.1 to about 0.9, such as about 0.2 mol/L or about 0.6 mol/L or about 0.7 mol/L. In one embodiment, the concentration of compound (9a) or (9b) in the solvent is about 0.01 to about 0.9 mol/L, for example, about 0.3 to about 0.7 mol/L, such as about 0.47 or 0.6 mol/L.

The compound (9a) or (9b), the cyanation reagent and the solvent may be added in any suitable order. In one embodiment, however, the compound (9a) or (9b) and cyanation reagent are combined with the solvent in a reaction vessel. In another embodiment, the compound (9a) or (9b) is charged to a reaction vessel with the solvent, followed by the addition of the cyanation reagent.

The reaction mixture may be heated to a temperature in the range from about 50 to about 200° C., such as in the range from about 60 to about 175° C., for example, about 100 to about 160° C. e.g. 150° C.

The reaction may be conducted under an inert atmosphere, such as argon or nitrogen.

The reaction is carried out for a period of time until it is determined that the reaction is substantially complete. Completion of the reaction may be determined by in-process analysis. Typically the reaction is complete within about 24 hours, and in some embodiments, within about 4 hours.

On completion of the reaction, the reaction mixture may be quenched (e.g. by adding it to a mixture of iron(III) chloride hexahydrate, water and hydrochloric acid), stirred for a period of time (e.g. about 2 hours) and extracted with a chlorinated solvent such as dichloromethane. The crude product may be recovered simply by evaporating the chlorinated solvent, whereupon it may be slurried in water and filtered. The compound of formula (20a) or (20b) may obtained in pure form by fractionally crystallising the crude material from toluene.

Howsoever the complex is recovered, the separated compound is preferably dried. Drying may be performed using known methods, for example, at temperatures in the range of about 10-60° C. and such as about 20-40° C. under 0.1-30 mbar for 1 hour to 5 days.

### Transition Metal Complexes of Formula (3)

In another aspect, the invention provides transition metal complexes of formula (3):

[MX(L¹)ₘ(L²)] (3)

wherein:
M is ruthenium, osmium or iron;
X is an anionic ligand;
L¹ is a monodentate phosphorus ligand, or a bidentate phosphorus ligand;
m is 1 or 2, wherein,
when m is 1, L¹ is a bidentate phosphorus ligand;
when m is 2, each L¹ is a monodentate phosphorus ligand; and
L² is a tridentate ligand of formula (2a) or (2b):

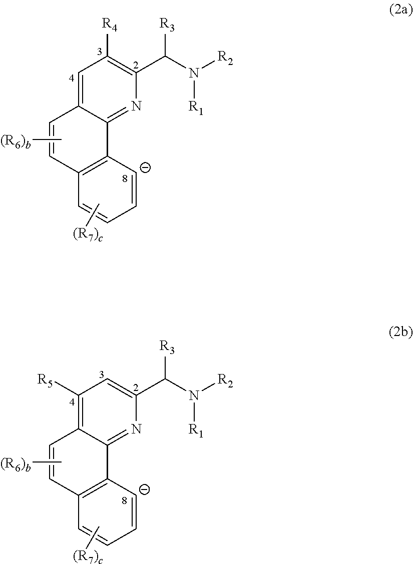

wherein:
R₁ and R₂ are independently selected from the group consisting of —H, —OH, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl;
R₃ is selected from the group consisting of —H, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl;
R₄ is selected from the group consisting of unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, substituted C₅₋₂₀-aryl;
R₅ is selected from the group consisting of unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, substituted C₅₋₂₀-aryl;
R₆ is selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″ and —CONR′R″, wherein R′ and R″ are independently selected from the group consisting of H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, substituted C₇₋₂₀-arylalkyl, or R′ and R″ together with the atom to which they are attached form a substituted or unsubstituted C₂₋₂₀-heterocycloalkyl group;
R₇ is selected from the group consisting of —H, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″ and —CONR′R″, wherein R′ and R″ are independently selected from the group consisting of H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, substituted C₇₋₂₀-arylalkyl, or R′ and R″ together with the atom to which they are attached form a substituted or unsubstituted C₂₋₂₀-heterocycloalkyl group;
b is an integer selected from 0, 1 or 2; and
c is an integer selected from 0, 1, 2 or 3.

M is a transition metal selected from the group consisting of ruthenium, osmium or iron. In one embodiment, M is ruthenium. When M is ruthenium, M may be Ru(II). In another embodiment, M is osmium. When M is osmium, M may be Os(II). In another embodiment, M is iron.

X is an anionic ligand and may be a coordinating or non-coordinating. In one embodiment, X is a coordinating anionic ligand. In another embodiment, X is a non-coordinating anionic ligand. The anionic ligand may be selected from the group consisting of halide, hydride (—H) or C₁₋₁₀-alkoxide (—O—C₁₋₁₀-alkyl). When the anionic ligand is a halide, the halide may be selected from the group consisting of —Cl, —Br and —I, for example, X is —Cl. In another embodiment, the anionic ligand may be a hydride (—H). In yet another embodiment, the anionic ligand may be an alkoxide selected from the group consisting of OMe, —OEt, —OPr (n- or i-), —OBu (n-, i- or t-).

L¹ is a phosphorus ligand. Any suitable phosphorus compound capable of forming a ligand-metal interaction with the M atom may be used. In the ligand, each phosphorus atom is covalently bonded to either 3 carbon atoms (tertiary phosphines) or to n heteroatoms and 3-n carbon atoms, where n=1, 2 or 3. Preferably, the heteroatom is selected from the group consisting of N and 0.

The ligand L¹ may be chiral or achiral, although in many instances it is preferred that the phosphorus ligand is chiral. A variety of chiral phosphorus ligands has been described and reviews are available, for example see W. Tang and X. Zhang, Chem Rev. 2003, 103, 3029-3070 and J. C. Carretero, Angew. Chem. Int. Ed., 2006, 45, 7674-7715.

When L¹ is a monodentate phosphorus ligand, m is 2. Each L¹ may be the same or different. Preferably, L¹ is a tertiary phosphine ligand PR₁₁R₁₂R₁₃. R₁₁, R₁₂ and R₁₃ may be independently selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. R₁₁, R₁₂ and R₁₃ may be independently substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more substituents such as halide (F, —Cl, —Br or —I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). Substituted or unsubstituted heteroaryl groups such as pyridyl may also be used. In an alternative embodiment, any two of R₁₁, R₁₂ and R₁₃ may be linked to form a ring structure with the phosphorus atom, preferably 4- to 7-membered rings. Preferably, R₁₁, R₁₂ and R₁₃ are the same and are phenyl i.e. PR₁₁R₁₂R₁₃ is triphenylphosphine. Alternatively, R₁₁, R₁₂ and R₁₃ may be the same and are tolyl i.e. PR₁₁R₁₂R₁₃ is tritolylphosphine (e.g. ortho-, meta- or para-tritolylphosphine).

Alternatively, L¹ is a bidentate phosphorus ligand and, in this instance, m is 1. Phosphorus ligands that may be used in the present invention include but are not restricted to the following structural types:

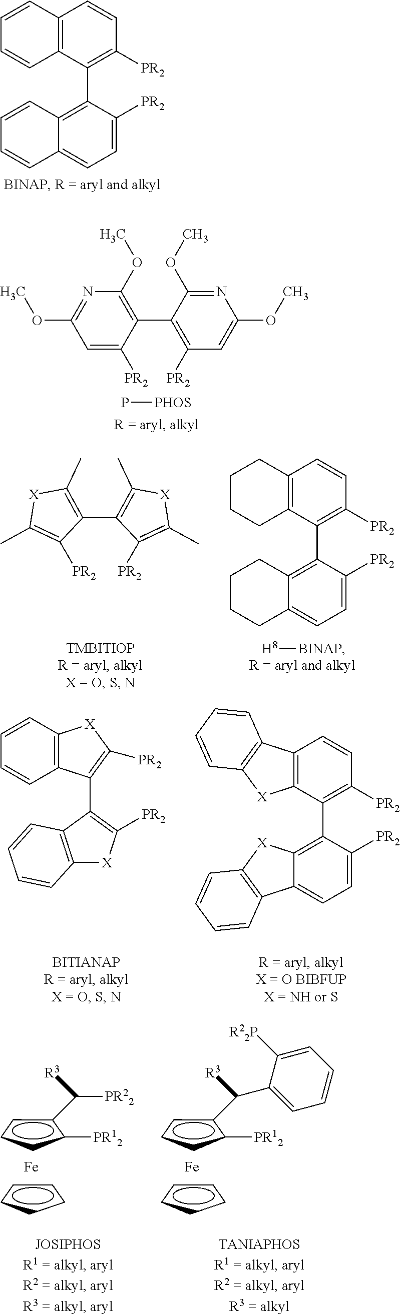

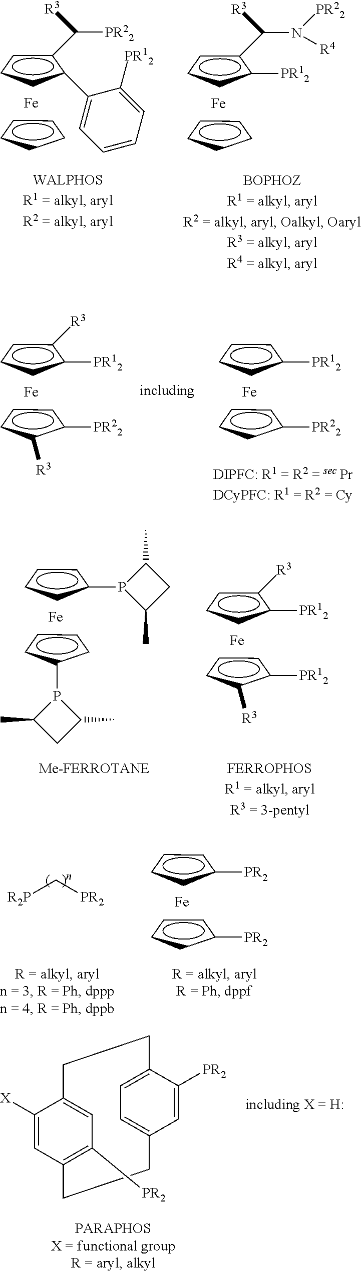

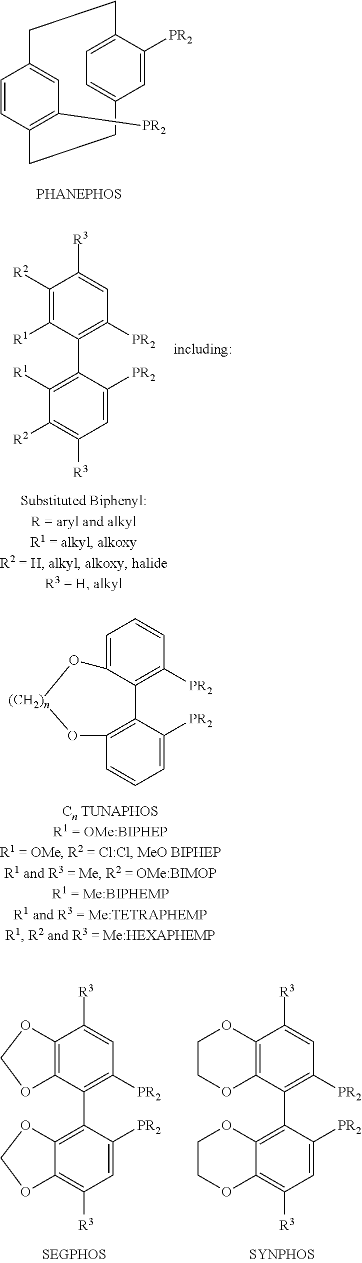

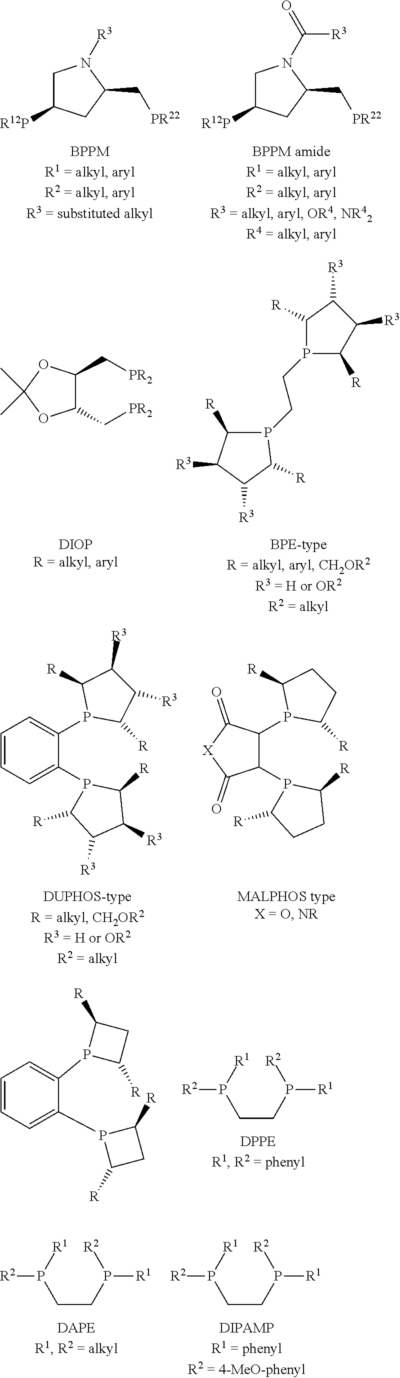

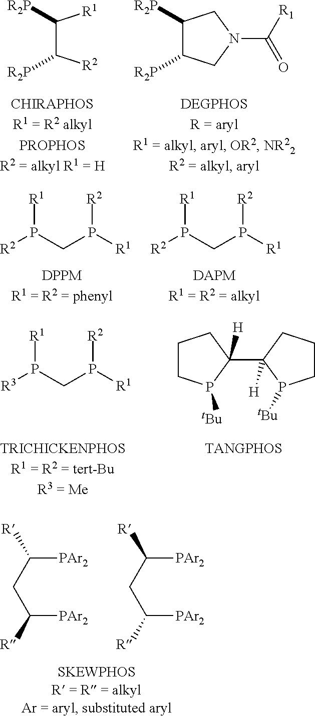

In the above structures —PR₂ may be —P(alkyl)₂ in which alkyl is preferably C₁-C₁₀ alkyl, —P(aryl)₂ where aryl includes phenyl and naphthyl which may be substituted or unsubstituted or —P(O-alkyl)₂ and —P(O-aryl)₂ with alkyl and aryl as defined above. PR₂ may also be substituted or unsubstituted P(heteroaryl)₂, where heteroaryl includes furanyl (e.g. 2-furanyl or 3-furanyl). —PR₂ is preferably either —P(aryl)₂ where aryl includes phenyl, tolyl, xylyl or anisyl or —P(O-aryl)₂. If —PR₂ is —P(O-aryl)₂, the most preferred O-aryl groups are those based on chiral or achiral substituted 1,1′-biphenol and 1,1′-binaphtol. Alternatively, the R groups on the P-atom may be linked as part of a cyclic structure.

Substituting groups may be present on the alkyl or aryl substituents in the phosphorus ligands. Such substituting groups are typically branched or linear C₁₋₆ alkyl groups such as methyl, ethyl, propyl, isopropyl, tert butyl and cyclohexyl.

The phosphorus ligands are preferably used in their single enantiomer form. These phosphorus ligands are generally available commercially and their preparation is known. For example, the preparation of PARAPHOS ligands is given in WO 04/111065, the preparation of Bophoz ligands in WO02/26750 and U.S. Pat. No. 6,906,212 and the preparation of Josiphos ligands in EP564406B and EP612758B.

The phosphorus ligand L¹ preferably includes Binap ligands, PPhos ligands, PhanePhos ligands, QPhos ligands, Josiphos ligands and Bophoz ligands.

When L¹ is a Binap ligand, the ligand may be of formula (1a) or (1b):

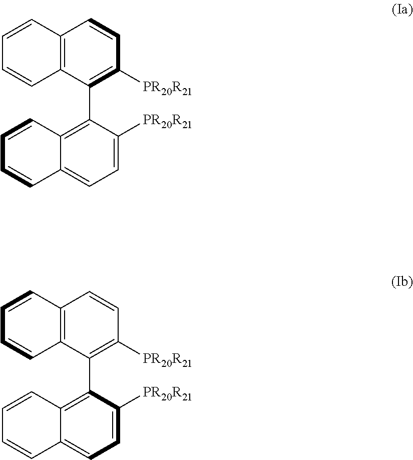

wherein,
R₂₀ and R₂₁ are each independently selected from the group consisting of unsubstituted C₃₋₂₀-cycloalkyl, substituted C₃₋₂₀-cycloalkyl, unsubstituted C₆₋₂₀-aryl and substituted C₆₋₂₀-aryl. In one embodiment, R₂₀ and R₂₁ are each independently selected from the group consisting of cycloalkyl groups such as cyclopropyl, cyclobutyl, cyclopentyl, cyclohexyl or adamantyl, or aryl groups such as phenyl, naphthyl or anthracyl. In one embodiment, the cycloalkyl groups may be optionally substituted with one or more substituents such as halide (—F, —Cl, —Br or —I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). Preferably, R₂₀ and R₂₁ are the same and are selected from the group consisting of phenyl, tolyl (o-, m- or p-, preferably p-tolyl) and xylyl (e.g. 3,5-xylyl).

When L¹ is a Josiphos ligand, the ligand may be of formula (IIa) or (IIb):

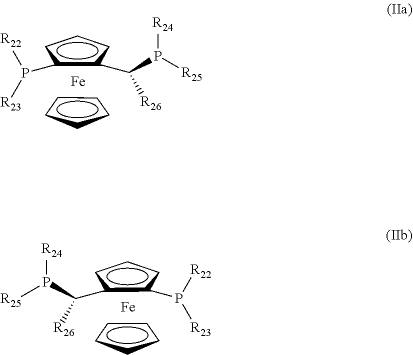

wherein,
R₂₂ and R₂₃ are independently selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl;
R₂₄ and R₂₅ are independently selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl; and
R₂₆ is selected from the group consisting of unsubstituted C₁₋₂₀-alkyl and substituted C₁₋₂₀-alkyl.

In one embodiment, R₂₂ and R₂₃ are independently selected from the group consisting of substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, aryl groups such as phenyl, naphthyl or anthracyl and heteroaryl groups such as furyl. In one embodiment, the alkyl groups may be optionally substituted with one or more substituents such as halide (—F, —Cl, —Br or —I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). The heteroaryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino or tri(halo)methyl (e.g. F₃C—). Preferably, R₂₂ and R₂₃ are the same and are selected from the group consisting of tert-butyl, cyclohexyl, phenyl, 3,5-bis(trifluoromethyl)phenyl, 4-methoxy-3,5-dimethylphenyl, 4-trifluoromethylphenyl, 1-naphthyl, 3,5-xylyl, 2-methylphenyl and 2-furyl, most preferably tert-butyl, cyclohexyl, phenyl, 3,5-bis(trifluoromethyl)phenyl, 4-methoxy-3,5-dimethylphenyl, 4-trifluoromethylphenyl, 1-naphthyl and 2-furyl.

In one embodiment, R₂₄ and R₂₅ are independently selected from the group consisting of substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, aryl groups such as phenyl, naphthyl or anthracyl and heteroaryl groups such as furyl. In one embodiment, the alkyl groups may be optionally substituted with one or more substituents such as halide (—F, —Cl, —Br or —I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). The heteroaryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino or tri(halo)methyl (e.g. F₃C—). Preferably, R₂₄ and R₂₅ are the same and are selected from the group consisting of tert-butyl, cyclohexyl, phenyl, 3,5-bis(trifluoromethyl)phenyl, 4-methoxy-3,5-dimethylphenyl, 4-trifluoromethylphenyl, 1-naphthyl, 3,5-xylyl, 2-methylphenyl and 2-furyl, most preferably tert-butyl, cyclohexyl, phenyl, 3,5-xylyl and 2-methylphenyl.

In one embodiment, R₂₆ is an unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl. Preferably, R₂₆ is methyl.

In one embodiment, the ligand of formula (IIa) is selected from the group consisting of:
(R)-1-[(S)-2-(diphenylphosphino)ferrocenyl]ethyldicyclohexylphosphine,
(R)-1-[(S)-2-(diphenylphosphino)ferrocenyl]ethyldi-tert-butylphosphine,
(R)-1-[(S)-2-(dicyclohexylphosphino)ferrocenyl]ethyldicyclohexylphosphine,
(R)-1-[(S)-2-(dicyclohexylphosphino)ferrocenyl]ethyldiphenylphosphine,
(R)-1-[(S)-2-(diphenylphosphino)ferrocenyl]ethyldi-3,5-xylylphosphine,
(R)-1-[(S)-2-(di-3,5-bis(trifluoromethyl)phenylphosphino)ferrocenyl]ethyldicyclohexylphosphine,
(R)-1-[(S)-2-(di-4-methoxy-3,5-dimethylphenylphosphino)ferrocenyl]ethyldicyclohexylphosphine,
(R)-1-[(S)-2-(di-3,5-bis(trifluoromethyl)phenylphosphino)ferrocenyl]ethyldi-3,5-xylylphosphine,
(R)-1-[(S)-2-(dicyclohexylphosphino)ferrocenyl]ethyldi-tert-butylphosphine,
(R)-1-[(S)-2-(di-(4-trifluoromethyl)phenylphosphino)ferrocenyl]ethyldi-tert-butylphosphine,
(R)-1-[(S)-2-(di-4-methoxy-3,5-dimethylphenylphosphino)ferrocenyl]ethyldi-tert-butylphosphine,
(R)-1-[(S)-2-(di-2-furylphosphino)ferrocenyl]ethyldi-3,5-xylylphosphine,
(R)-1-[(S)-2-(di-2-furylphosphino)ferrocenyl]ethyldi-tert-butylphosphine,
(R)-1-[(S)-2-(di-1-naphthylphosphino)ferrocenyl]ethyldi-tert-butylphosphine,
(R)-1-[(S)-2-(di-1-naphthylphosphino)ferrocenyl]ethyldi-3,5-xylylphosphine,
(R)-1-[(S)-2-(di-4-methoxy-3,5-dimethylphenylphosphino)ferrocenyl]ethyldi-3,5-xylylphosphine,
(R)-1-[(S)-2-(di-4-methoxy-3,5-dimethylphenylphosphino)ferrocenyl]ethyldi-(2-methylphenyl)phosphine,
(R)-1-[(S)-2-(di-2-furylphosphino)ferrocenyl]ethyldi-(2-methylphenyl)phosphine,
(R)-1-[(S)-2-(di-tert-butylphosphino)ferrocenyl]ethyldiphenylphosphine,
(R)-1-[(S)-2-(di-tert-butylphosphino)ferrocenyl]ethyldi-(2-methylphenyl)phosphine,
(R)-1-[(S)-2-(diphenylphosphino)ferrocenyl]ethyldiphenylphosphine,
(R)-1-[(S)-2-(diphenylphosphino)ferrocenyl]ethyldi(adamantyl)phosphine, and
(R)-1-[(S)-2-(di(adamantyl)phosphino)ferrocenyl]ethyldiphenylphosphine.

In one embodiment, the ligand of formula (IIb) is selected from the group consisting of:
(S)-1-[(R)-2-di(phenylphosphino)ferrocenyl]ethyldicyclohexylphosphine,
(S)-1-[(R)-2-di(phenylphosphino)ferrocenyl]ethyldi-tert-butylphosphine,
(S)-1-[(R)-2-di(cyclohexylphosphino)ferrocenyl]ethyldicyclohexylphosphine,
(S)-1-[(R)-2-di(cyclohexylphosphino)ferrocenyl]ethyldiphenylphosphine,
(S)-1-[(R)-2-di(phenylphosphino)ferrocenyl]ethyldi-3,5-xylylphosphine,
(S)-1-[(R)-2-di-(3,5-bis(trifluoromethyl)phenylphosphino)ferrocenyl]ethyldicyclohexylphosphine,
(S)-1-[(R)-2-di-(4-methoxy-3,5-dimethyl)phenylphosphino)ferrocenyl]ethyldicyclohexylphosphine,
(S)-1-[(R)-2-di-(3,5-bis(trifluoromethyl)phenylphosphino)ferrocenyl]ethyldi-3,5-xylylphosphine,
(S)-1-[(R)-2-di(cyclohexylphosphino)ferrocenyl]ethyldi-tert-butylphosphine,
(S)-1-[(R)-2-di-((4-trifluoromethyl)phenylphosphino)ferrocenyl]ethyldi-tert-butylphosphine,
(S)-1-[(R)-2-di-(4-methoxy-3,5-dimethyl)phenylphosphino)ferrocenyl]ethyldi-tert-butylphosphine,
(S)-1-[(R)-2-di-(2-furyl)phosphino)ferrocenyl]ethyldi-3,5-xylylphosphine,
(S)-1-[(R)-2-di-(2-furyl)phosphino)ferrocenyl]ethyldi-tert-butylphosphine,
(S)-1-[(R)-2-di(1-naphthyl)phosphino)ferrocenyl]ethyldi-tert-butylphosphine,
(S)-1-[(R)-2-di(1-naphthyl)phosphino)ferrocenyl]ethyldi-3,5-xylylphosphine,
(S)-1-[(R)-2-di-(4-methoxy-3,5-dimethyl)phenylphosphino)ferrocenyl]ethyldi-3,5-xylylphosphine,
(S)-1-[(R)-2-di-(4-methoxy-3,5-dimethyl)phenylphosphino)ferrocenyl]ethyldi-(2-methylphenyl)phosphine,
(S)-1-[(R)-2-di-(2-furyl)phosphinoferrocenyl]ethyldi-(2-methylphenyl)phosphine,
(S)-1-[(R)-2-di(tert-butylphosphino)ferrocenyl]ethyldiphenylphosphine,
(S)-1-[(R)-2-di(tert-butylphosphino)ferrocenyl]ethyldi-(2-methylphenyl)phosphine,
(S)-1-[(R)-2-diphenylphosphinoferrocenyl]ethyldiphenylphosphine,
(S)-1-[(R)-2-(diphenylphosphino)ferrocenyl]ethyldi(adamantyl)phosphine, and
(S)-1-[(R)-2-(di(adamantyl)phosphino)ferrocenyl]ethyldiphenylphosphine.

In one preferred embodiment, the ligand of formula (IIa) is (R)-1-[(S)-2-diphenylphosphinoferrocenyl]ethyldiphenylphosphine. In another preferred embodiment, the ligand of formula (IIb) is (S)-1-[(R)-2-diphenylphosphinoferrocenyl]ethyldiphenylphosphine.
The phosphorus ligand L¹ also preferably includes PPh₃, PCy₃ (tricyclohexylphosphine), dppf (1,1′-bis(diphenylphosphino)ferrocene), dppp (1,3-bis(diphenylphosphino)propane), dppb (1,4-bis(diphenylphosphino)butane), Dipfc (1,1′-bis(di-isopropylphosphino)ferrocene), dCyPfc (1,1′-bis(di-cyclohexylphosphino)ferrocene and DB^{t}PF (1,1′-bis(di-tert-butylphosphino)ferrocene). In one embodiment, the phosphorus ligand L¹ is unsubstituted. In another embodiment, the ligand L¹ is substituted.

Particularly preferred phosphorus ligands L¹ may be selected from the group consisting of dppf, dppp and dppb.

L² is a CNN tridentate ligand of formula (2a) or (2b), each comprising a carbon-M bond, a pyridinyl group and an amino group. The ligands are tridentate as they each coordinate to the M atom via:
a) a carbon-M bond (at C-8). The carbon-M bond is a carbon-metal bond created by orthometallation during the synthesis of the [MX(L¹)ₘ (L²)] complex of formula (3);
b) the nitrogen atom of the pyridinyl ring; and
c) the nitrogen atom of the amino group.

In one embodiment, L² is a tridentate ligand of formula (2a). In another embodiment, L² is a tridentate ligand of formula (2b).

R₁, R₂, R₃, R₄, R₅, R₆, R₇ and b are as generally described above.

R₇ may be present or absent. When absent, c is 0 i.e. the aryl ring is unsubstituted. When R₇ is present, c may be 1, 2 or 3. When c is 2 or 3, each R₇ may be the same or different to each other. The or each R₇ are as generally described above. In one preferred embodiment, c is 0 i.e. R₇ is absent.

### Preparation of the Complex of Formula (3)

The complex of formula (3) may be prepared by reacting a suitable transition metal complex, a ligand L¹, a compound of formula (1a) or (1b) or salts thereof, and a base in an alcohol solvent, provided C-8 of the compound of formula (1a) or (1b) is —H.

The compound of formula (1a) or salts thereof, the compound of formula (1b) or salts thereof and the ligand L¹ are as generally described above.

The ligand L¹ may be present in stoichiometric or greater quantities to the compound (1a) or (1b), or salt thereof. When the free base of compound (1a) or (1b) is reacted, stoichiometric or slight excess of L¹ may be suitable, for example, about 1:1.1 to 1:1.5 molar ratio of compound (1a) or (1b) to L¹.

The transition metal complex may be selected from the group consisting of [ruthenium (arene) (halogen)₂]₂, [ruthenium (halogen) (P(unsubstituted or substituted aryl)₃)], [osmium (arene) (halogen)₂], [osmium (halogen)₂ (P(unsubstituted or substituted aryl)₃)] and [osmium (N(unsubstituted or substituted alkyl)₃)₄ (halogen)₂].

The arene may be an unsubstituted or substituted benzene wherein the substituents are selected from chain C₁₋₆ alkyl, C₁₋₆ alkoxy, C₁₋₆ carboalkoxy, —OH or NO₂. In one embodiment, the arene may be selected from the group consisting of benzene, cymene, toluene, xylene, trimethylbenzene, hexamethylbenzene, ethylbenzene, t-butylbenzene, cumene (isopropylbenzene), anisole (methoxybenzene), methylanisole, chlorobenzene, dichlorobenzene, trichlorobenzene, bromobenzene, fluorobenzene, methylbenzoate and methyl methyl benzoate (e.g. methyl 2-methylbenzoate). In another embodiment, the arene is benzene, p-cymene or mesitylene (1,3,5-trimethylbenzene).

The halogen may be selected from the group consisting of chlorine, bromine and iodine, e.g. chlorine.

The P(unsubstituted or substituted aryl)₃ may be a P(substituted aryl)₃ or a P(unsubstituted aryl)₃. Examples of P(substituted aryl)₃ and P(unsubstituted aryl)₃ include but are not limited to PPh₃ or P(Tol)₃, where the tolyl group may be ortho-, para- or meta-substituted.

The N(unsubstituted or substituted alkyl)₃ may be a N(substituted alkyl)₃ or a N(unsubstituted alkyl)₃ (such as NEt₃).

In one embodiment, the [ruthenium (halogen) (P(unsubstituted or substituted aryl)₃)] may be RuCl₂PPh₃ or RuCl₂(P(o-Tol)₃). In one embodiment, the [osmium (halogen)₂ (P(unsubstituted or substituted aryl)₃)] may be OsCl₂PPh₃ or OsCl₂(P(o-Tol)₃).

In one embodiment, the [ruthenium (arene) (halogen)₂]₂ may be [RuCl₂(p-cymene)]₂, [RuCl₂(benzene)]₂ or [RuCl₂(mesitylene)]₂. In one embodiment, the [osmium (arene) (halogen)₂] may be [OsCl₂(p-cymene)], [OsCl₂(benzene)] or [OsCl₂(mesitylene)]

In one embodiment, the [osmium (N(unsubstituted or substituted alkyl)₃)₄ (halogen)₂] may be [(Et₃N)₄OsCl₂].

In the presence of a suitable base and when a hydrogen atom is present at C-8 of the compounds (1a) and (1b), the compounds (1a) and (1b) orthometallate with the transition metal atom (e.g. Ru or Os) to form a transition metal complex comprising the CNN-tridentate ligands (2a) and (2b). R₁, R₂, R₃, R₄, R₅, R₆, R₇ and b are as generally described above and c may be 0, 1, 2 or 3 (but not 4).

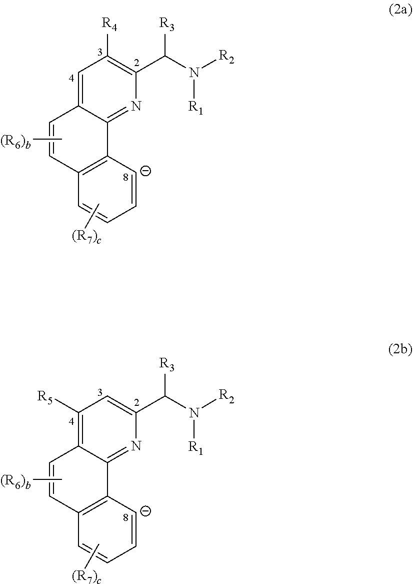

The base may be any suitable base which is capable of removing the hydrogen at C-8 in the compounds (1a) or (1b). Examples of bases include trialkylamines (such as triethylamine), pyridine, dimethylpyridine (e.g. 2,6-, 2,3-, 3,5-, 2,5- or 3,4-dimethylpyridine), alkali metal hydroxides (such as sodium hydroxide or potassium hydroxide) or alkali metal alkoxides (such as sodium methoxide or potassium methoxide).

The base may be present in stoichiometric or greater quantities to the compound (1a) or (1b), or salt thereof. When the free base of compound (1a) or (1b) is reacted, stoichiometric or slight excess of base may be suitable, for example, about 1:1.1 to 1:1.5 molar ratio of compound (1a) or (1b) to base. When salts of compound (1a) or (1b) are utilised, however, excess base is generally required in order to form the free base of the compound (1a) or (1 b) from the salt of compound (1a) and (1b), and deprotonate the compound (1a) or (1 b) at C-14 to form the ligand (2a) or (2b). In this respect, the molar ratio of the salts of compound (1a) or (1b) to base may be about 1:5 to about 1:20, such as about 1:7.5 to about 1:15, such as about 1:10.

Any suitable alcohol solvent may be utilised. Suitable alcohols have boiling points at atmospheric pressure (i.e. 1.0135×105 Pa) below 120° C., more preferably below 110° C. and even more preferably below 100° C. Preferably the alcohol is dry. The alcohol solvent may be selected from the group consisting of methanol, ethanol, isopropanol and mixtures thereof. In one embodiment, the alcohol solvent is iso-propanol (i.e. 2-propanol).

The concentration of the transition metal complex in the solvent may be about 0.001 mol/L to about 10.0 mol/L, such as about 0.01 to about 1.0 mol/L, for example, about 0.02 mol/L to about 0.5 mol/L.

In combining the transition metal complex, the ligand L¹, the ligand L² and base in the alcohol, the components may be mixed in any suitable order, although, in one embodiment, the transition metal complex and ligand L¹ are slurried or suspended in the alcohol solvent, followed by the addition of the ligand L² and the base. After the transition metal complex and the ligand L¹ are combined with the alcohol, the reaction mixture may be stirred and heated (e.g. at reflux) for a period of time (e.g. for up to 2-3 hours). The mixture may be stirred for a period e.g. preferably 1 minute to 3 hours, more preferably 2 minutes to 2 hours and most preferably 2.5 minutes to 1.5 hours. The ligand L² and the base may then be added to the reaction mixture and the reaction mixture stirred and heated (e.g. at reflux) for a further period of time (e.g. for up to 5-6 hours).

The reaction may be conducted under an inert atmosphere, such as nitrogen or argon.

The reaction mixture may be treated with an alkane (such as pentane, hexane or heptane) which causes the complex (3) to precipitate or crystallise. The solid complex (3) may be recovered directly by filtering, decanting or centrifuging. If desired a proportion of the alcohol/alkane solvent mixture may be evaporated prior to the recovery of the complex.

Alternatively, the solid complex (3) may be recovered simply by evaporating the alcohol/alkane solvent mixture.

Howsoever the complex is recovered, the separated complex is preferably dried. Drying may be performed using known methods, for example, at temperatures in the range of about 10-60° C. and such as about 20-40° C. under 0.1-30 mbar for 1 hour to 5 days. It may be desirable to store the complex under conditions which substantially excludes light.

The complexes prepared by the processes of the present invention are pure and may be used in catalytic applications as obtained or further dried. The methods are suited to large-scale manufacture and large-scale catalytic applications.

### Methods of Catalysis

In one aspect of the invention there is provided the use of a complex of formula (3) as a catalyst, for example in a hydrogenation reaction or a transfer hydrogenation reaction. Such reactions may be broadly referred to as hydrogen reduction reactions. It is envisaged that the complexes may also be used in deuteration reactions, tritiation reactions, the isomerization of allylic alcohols, dehydrogenation reactions which may be carried out with or without a hydrogen acceptor (e.g. the dehydrogenation of alcohols to aldehydes or ketones, or the dehydrogenation of alcohols to esters), the reduction of the alkenyl bond in α,ß-unsaturated carbonyls and in “hydrogen borrowing” reactions (which include dehydrogenation and hydrogenation steps, e.g. the alkylation of amines with alcohols). The complex of formula (3) is as described above.

In one embodiment, the method comprises the step of reacting a substrate comprising a carbon-oxygen double bond in the presence of a complex of formula (3).

In one embodiment, the reaction is a hydrogenation reaction, and the method includes reacting the substrate with hydrogen gas in the presence of a complex of formula (3). The reaction may further comprise an alkali metal alkoxide (such as i-PrONa).

In one embodiment, the reaction is a deuteration reaction, and the method includes reacting the substrate with deuterium gas in the presence of a complex of formula (3). The reaction may further comprise an alkali metal alkoxide (such as i-PrONa).

In one embodiment, the reaction is a tritiation reaction, and the method includes reacting the substrate with tritium gas in the presence of a complex of formula (3). The reaction may further comprise an alkali metal alkoxide (such as i-PrONa).

In one embodiment, the reaction is a transfer hydrogenation, and the method includes reacting the substrate with a hydrogen donor in the in the presence of a complex of formula (3). The hydrogen donor may be selected from formic acid, a formic acid alkali metal salt, and an alcohol, such as an alcohol having a hydrogen atom at a carbon atom that is a to the carbon atom to which the alcohol group is attached, such as iso-propanol. The reaction may further comprise an alkali metal alkoxide (such as i-PrONa). In one embodiment, the substrate may be an aldehyde and the hydrogen donor may be ammonium formate. In this instance, the aldehyde is reduced to a primary alcohol. As used herein, a hydrogen donor is not gaseous hydrogen.

Examples of compounds containing a carbon-oxygen double bond include ketones, aldehydes, esters and lactones, amongst others.

The method may include the step of reducing a substrate, for example the hydrogenation of a carbonyl-containing substrate to yield the corresponding alcohol.

A suitable substrate to be hydrogenated includes, but is not limited to, a carbonyl of formula (1):

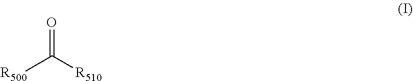

wherein,
R₅₀₀ and R₅₁₀ are each independently selected from the group consisting of hydrogen, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₃₋₂₀-cycloalkoxy, substituted C₃₋₂₀-cycloalkoxy, unsubstituted C₂₋₂₀-alkenyl, substituted C₂₋₂₀-alkenyl, unsubstituted C₄₋₂₀-cycloalkenyl, substituted C₄₋₂₀-cycloalkenyl, unsubstituted C₂₋₂₀-alkynyl, substituted C₂₋₂₀-alkynyl, unsubstituted C₆₋₂₀-aryl, substituted C₆₋₂₀-aryl, unsubstituted C₁₋₂₀-heteroalkyl, substituted C₁₋₂₀-heteroalkyl, unsubstituted C₂₋₂₀-cycloheteroalkyl, substituted C₂₋₂₀-cycloheteroalkyl, unsubstituted C₃₋₂₀-heteroaryl, substituted C₃₋₂₀-heteroaryl, —NR₆₀₀R₆₁₀, —COR₆₀₀, —COOR₆₀₀, —CONR₆₀₀R₆₁₀, unsubstituted —C₁₋₂₀-alkyl-COOR₆₀₀, substituted —C₁₋₂₀-alkyl-COOR₆₀₀, unsubstituted —C₁₋₂₀-alkyl-COR₆₀₀, substituted —C₁₋₂₀-alkyl-COR₆₀₀, unsubstituted —C₁₋₂₀-alkyl-CONR₆₀₀R₆₁₀, substituted —C₁₋₂₀-alkyl-CONR₆₀₀R₆₁₀, unsubstituted —C₂₋₂₀-alkynyl-C₆₋₂₀-aryl, substituted —C₂₋₂₀-alkynyl-C₆₋₂₀-aryl, unsubstituted C₂₋₂₀-alkynyl-C₁₋₂₀-alkyl, substituted C₂₋₂₀-alkynyl-C₁₋₂₀-alkyl; or
R₅₀₀ and R₅₁₀ are bound by an unsubstituted C₁₋₂₀ alkyl, substituted C₁₋₂₀ alkyl, unsubstituted C₁₋₂₀ alkoxy, substituted C₁₋₂₀ alkoxy, unsubstituted C₂₋₂₀ alkenyl or substituted C₂₋₂₀ alkenyl; or
R₅₀₀ and R₅₁₀ are bound to form a 5, 6 or 7 membered ring by an unsubstituted —(CH₂)ₜ-(ortho-C₅₋₆-aryl)-(CH₂)ᵤ— chain, substituted —(CH₂)ₜ-(ortho-C₅₋₆-aryl)-(CH₂), chain, unsubstituted —(CH₂)ₜ-(ortho-C₅₋₆-aryl)-L^{Q}-(CH₂)ᵤ— chain, substituted —(CH₂)ₜ-(ortho-C₅₋₆-aryl)-L⁴-(CH₂), chain, unsubstituted —(CH₂)ₜ-(ortho-C₅₋₆-heteroaryl)-(CH₂)ᵤ— chain or substituted —(CH₂)ₜ-(ortho-C₅₋₆-heteroaryl)-(CH₂), chain;
wherein t is an integer selected from 0 or 1,
u is an integer selected from 2, 3 or 4,
-L^{Q}- is selected from the group consisting of —O—, —N— and —SO₂—,
wherein the substituents are selected from the group consisting of unsubstituted unsubstituted C₃₋₂₀-cycloalkyl, unsubstituted C₁₋₂₀-alkoxy, unsubstituted C₃₋₂₀-cycloalkoxy, unsubstituted C₆₋₂₀-aryl, unsubstituted C₆₋₂₀ aryloxy, unsubstituted C₁₋₂₀-heteroalkyl, unsubstituted C₂₋₂₀-cycloheteroalkyl, unsubstituted C₃₋₂₀-heteroaryl, straight or branched tri-C₁₋₂₀-alkylsilyl-, -Hal, —OH, —CN, —NR₆₀₀R₆₁₀, —COR₆₀₀, —COOR₆₀₀, —CONR₆₀₀R₆₁₀ and —CF₃,
wherein R₆₀₀ and R₆₁₀ are independently selected from the group consisting of hydrogen, unsubstituted C₁₋₂₀-alkyl, unsubstituted C₃₋₂₀-cycloalkyl, unsubstituted C₁₋₂₀-alkoxy, unsubstituted C₃₋₂₀-cycloalkoxy, unsubstituted C₆₋₂₀-aryl, unsubstituted C₆₋₂₀ aryloxy and —OH.

In one embodiment, R₅₀₀ and R₅₁₀ are not both hydrogen.

In one embodiment, one of R₅₀₀ and R₅₁₀ is hydrogen and the other of R₅₀₀ and R₅₁₀ is selected from the groups described above i.e. the carbonyl of formula (I) is an aldehyde.

In one embodiment, R₅₀₀ and R₅₁₀ are independently selected from the groups described above provided that neither R₅₀₀ or R₅₁₀ are hydrogen i.e. the carbonyl of formula (I) is a ketone.

The reaction may be a non-asymmetric or asymmetric reduction reaction.

When R₅₀₀ and/or R₅₁₀ are different, the compounds of formula (I) are prochiral when the compound of formula (I) is an aldehyde or ketone. In this instance, the hydrogenation catalysed by the complex of formula (3) may be enantioselective when the phosphorus ligand L¹ or the ligand L² is chiral.

The enantiomeric excess may be greater than 80% ee. In certain embodiments, the enantiomeric excess may be greater than 85% ee, in certain embodiments greater than 90% ee, in certain embodiments greater than 93% ee.

The reaction conditions for the reduction reactions are not particularly limited, and may be performed at the temperatures, pressures, concentrations that are appropriate to maximise the yield and stereoselectivity of the reaction, whilst minimising reaction time and reaction impurities.

Example reaction conditions for transfer hydrogenation reactions are described in WO2009/007443, the contents of which are hereby incorporated by reference.

After the reduction reaction is deemed complete, the reaction mixture may be at least partially separated, for example to isolate the product, and/or to isolate the complex. In a stereoselective reaction the product may be isolated from undesired stereoisomers.

The complexes of the invention may be separated from the reaction mixture by precipitation, for example following the addition of an anti-solvent to the reaction mixture or following the concentration of the reaction mixture.

The methods described above may be performed under an inert atmosphere, such as an argon or nitrogen atmosphere.

### Other Preferences

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be described by the way of the following non-limiting Examples.

### EXAMPLES

All reactions were carried out under argon or nitrogen atmosphere. Anhydrous THF, toluene, MeOH, 2-propanol were purchased from Aldrich and absolute EtOH was purchased from VWR. The bisphosphines dppp, dppb, dppf and rac-BINAP were purchased from Alfa Aesar (Johnson Matthey), whereas (S,R)-JOSIPHOS was purchased from STREM. RuCl₂(PPh₃)₃ and [RuCl₂(p-cymene)]₂ used were commercial grade products from Johnson Matthey. NMR measurements were recorded on Bruker AC 200 and Bruker Advance 400 spectrometers and the chemical shifts, in ppm, are relative to TMS for ¹H and ¹³C{¹H}, and 85% H₃PO₄ for ³¹P{¹H}. High-resolution mass spectra (HRMS) were acquired on a Bruker BioApex II 4.7e FTICR mass spectrometer, whereas the GC analysis was performed with a Varian GP-3380 gas chromatograph equipped with a MEGADEX-ETTBDMS-β chiral column.

### Abbreviations

AMPY 2-(aminomethyl)pyridine
DCM dichloromethane
DMF dimethylformamide
dppp 1,3-bis(diphenylphosphino)propane
dppb 1,4-bis(diphenylphospino)butane
dppf 1,1′-bis(diphenylphosphino)ferrocene

### (S,R)-JOSIPHOS (S)-1-{(R)-2-[diphenylphosphine]ferrocenyl}ethyldicyclohexylphosphine

eq. equivalent
h hour
HY hydrogenation

### L Litre

mL millilitre

### RT Room Temperature

TH transfer hydrogenation

### Example 1

### Synthesis of N-(naphthalen-1-yl)-3-oxo-3-phenylpropanamide (1)

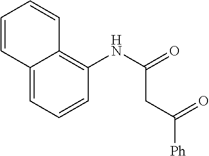 [SMILES: O=C(CC(=O)c1ccccc1)Nc1cccc2ccccc12]

The 1-naphthylamine reagent used might have contained a few ppm quantity of the highly carcinogenic 2-naphtylamine. While the 1-naphthylamine reagent had a quality allowing its use, 2-naphtylamine is banned from use in Europe and many other countries. An occupational health assessment required that in order to minimise exposure the N-(naphthalen-1-yl)-3-oxo-3-phenylpropanamide 1 should be assayed and characterised as a crude product and then converted on as described in Example 2. 1-Naphthylamine (1183 g, 8.26 mol) and xylene (isomer mixture, 10 L) was charged to a 20 L round bottom flask, equipped with a distillation setup that allowed distillation of the reaction side product ethanol as azeotrope with xylene. The reaction was heated at an oil bath temperature of 160° C. Ethyl benzoylacetate (1775 g, 9.23 mol) was added over 1.5 hours, resulting in a steady distillation of ethanol/xylene. After completion of the addition, the reaction temperature was kept at 160° C. (oil bath) for two hours and then allowed to cool to 120° C. At this temperature the reaction solvent was distilled by vacuum distillation. The resulting brown solid was cooled to room temperature and slurried in n-heptane (9 L).

The slurry was filtered, the solid product further washed with 1 L of heptane and dried under vacuum in a desiccator (over KOH) at 40° C. to afford the pale brown solid 1, 1929 g, 81% yield. The product may be used in the next step without further purification. ¹H NMR (400 MHz, CDCl₃): δ 10.03 (br s, 1H, NH), 8.05 (d, 1H, J=7.6), 8.00 (d, 2H, J=7.8), 7.79 (d, 1H, J=7.8), 7.77 (d, 1H, J=8.3), 7.68-7.58 (m, 1H), 7.55-7.35 (m, 6H), 4.18 (s, 2H) (complex spectrum, only major resonances given). ¹³C{¹H} NMR (100.61 MHz, CDC₃,): δ 197.31 (C C═O), 164.3 (C C═O amide), 136.11, 134.51, 134.10, 132.39, 129.05, 128.71, 128.63, 128.36, 128.71, 127.93, 126.51, 125.78, 125.47, 120.8, 119.72, 44.9 (complex spectrum, only major resonances given).

### Example 2

### Synthesis of 4-phenylbenzo[h]quinolin-2(1H)-one (2)

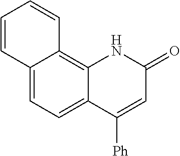 [SMILES: O=c1cc(-c2ccccc2)c2ccc3ccccc3c2[nH]1]

To crushed ice (1608 g) in a 20 L round bottom flask with efficient overhead stirring was added cautiously 4828 g of 98% sulphuric acid. At the end of the addition, the mixture had a temperature (internal) of 80° C. N-(naphthalen-1-yl)-3-oxo-3-phenylpropanamide (1, 1929 g, as produced in example 1) was added as solid in portions over 30 minutes. After the addition had completed, the mixture had a temperature (internal) of 49° C. The reaction was then carefully heated in an oil bath, set to 100° C. A very thick slurry of purple solid had formed after 5 hours at this temperature and the mixture was allowed to cool to room temperature. 3 L of cold water was added with external cooling by crushed ice and the mixture was stirred for three hours. The slurry was filtered and the solid purple product washed with 3 L of water and sucked dry as much as possible. The product was then transferred to a 10 L flask and stirred with 6 L of acetone for 30 minutes. The slurry was again filtered and the solid washed with 4×1 L of acetone. The pale brown purple solid was dried in a desiccator over KOH at 40° C. to afford the solid product 2 (1571 g, 87% yield, 70% in two steps from 1-naphtylamine used in Example 1). The product may be used in the next step without further purification. HRMS found: [M+H]⁺ 272.1058. calcd for C₁₉H₁₄NO: 272.1070. ¹H NMR (400 MHz, DMSO-d⁶): δ 12.26 (s, br, NH) 8.94 (1H, d, J=7.9 Hz), 7.97 (1H, d, J=6.9 Hz), 7.72-7.62 (2H, m), 7.60-7.46 (6H, m), 7.40 (1H, d, J=8.8 Hz), 6.54 (1H, s). ¹³C{¹H} NMR (100.61 MHz, DMSO-d⁶): δ 162.34, 152.95, 137.66, 134.06, 129.26, 129.20, 128.80, 128.66, 127.13, 123.46, 123.10, 122.65 (the solubility of the compound is so low that only the 12 non quaternary carbons are visible

### Example 3

### Synthesis of 2-bromo-4-phenylbenzo[h]quinoline (3)

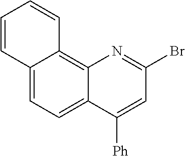 [SMILES: Brc1cc(-c2ccccc2)c2ccc3ccccc3c2n1]

The product obtained in example 2, 1571 g of 4-phenylbenzo[h]quinolin-2(1H)-one (2, 5.79 mol) was dissolved in 7.8 L of toluene and azeotropically dried using Dean-Stark distillation. At room temperature 1660 g of POBr₃ was carefully added in portions. After the reaction mixture was heated overnight at 120° C. it was cooled to room temperature. This mixture was added to 10 L of water and aqueous concentrated NaOH was added until a pH=14 was measured in the water phase. At this stage the reaction mixture had to be filtered over Celite to remove a very fine, very insoluble impurity. The Celite pad was washed with several 1 L quantities of toluene. The organic filtrate was stripped to dryness and the residue recrystallized from isopropyl alcohol to afford the product 3 as a brown powder (1392 g, 72% yield). This batch was assayed for water content and 0.06% wt/wt residual water content was determined. HRMS found: [M+H]⁺ 334.0221. calcd for C₁₉H₁₃BrN: 334.0226. ¹H NMR (400 MHz, CDCl₃): δ 9.19 (1H, d, J=7.7 Hz), 7.79 (1H, d, J=7.5 Hz), 7.70-7.57 (4H, m), 7.51 (1H, s), 7.48-7.37 (5H, m). ¹³C{¹H} NMR (100.61 MHz, CDCl₃): δ 150.91, 147.61, 140.50, 137.05, 133.60, 130.70, 129.60, 129.55, 128.86, 128.81, 128.73, 128.03, 127.58, 127.32, 126.20, 125.24, 123.52, 122.64.

### Example 4

### Synthesis of 4-phenyl-benzo[h]quinoline-2-carbaldehyde (4)

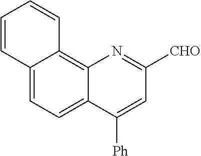 [SMILES: O=Cc1cc(-c2ccccc2)c2ccc3ccccc3c2n1]

33.1 g of 2-bromo-4-phenylbenzo[h]quinoline (3, 0.1 mol) was dissolved in dry THF (300 mL) in a 1 L three neck round bottom flask and the mixture was cooled to −75° C. (IPA/dry ice bath). 45 mL of 2.5M n-butyl lithium in hexanes (0.1125 mol, 1.125 eq) was added slowly so that the internal temperature never went above −70° C. After stirring the reaction for another one hour at −75° C., 11 g of anhydrous dimethylformamide (0.15 mol, 1.5 eq) was added in small drops so that the internal temperature never went above −65° C. The reaction was then allowed to reach room temperature overnight. The next day, 100 mL of water was added to quench the reaction, followed by 15 mL of glacial acetic acid. The organic layer was separated and washed with 50 ml of saturated sodium chloride solution. It was then dried over sodium sulphate. The filtrate after removal of the sodium sulphate was concentrated to dryness. The residue was treated with 75 mL of ethanol and the resulting slurry filtered to obtain the product 4, which is dried under vacuum. Yield 20.0 g (70.6%). HRMS found: [M+H]⁺ 284.1073. calcd for C₂₀H₁₄NO: 284.1070. ¹H NMR (400 MHz, CDCl₃): δ 10.41 (1H, s), 9.50 (1H, d, J=7.7 Hz), 8.12 (1H, s), 7.93 (1H, d, J=7.7 Hz), 7.89 (2H, s), 7.85-7.75 (7H, m). ¹³C{¹H} NMR (100 MHz, CDCl₃): δ 194.3, 150.5, 149.5, 147.0, 137.8, 133.5, 131.7, 130.5, 129.7, 129.0, 128.7, 128.2, 127.8, 127.0, 125.0, 124.0, 122.8, 118.9.

In a second reaction, 2-bromo-4-phenylbenzo[h]quinoline (21.56 g, 64.51 mmol) in less solvent (90 mL of dry THF) was reacted as above at −78° C. with 32.3 mL n-BuLi (2.5 M in hexane, 80.63 mmol, 1.25 eq.), then with dry DMF (6.29 mL, 80.63 mmol, 1.25 eq). After a workup similar to above, 19.35 g of impure product was obtained and used without further purification for the synthesis of 5.

### Example 5

### Synthesis of 2-carbaldehyde-4-phenylbenzo[h]quinoline oxime hydrochloride (5)

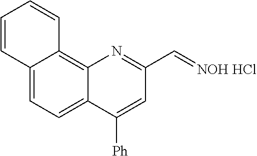

The crude aldehyde 4 from the second reaction above (19.35 g, 68.3 mmol) was slurried in absolute ethanol (240 mL) and heated at 40° C. Hydroxylamine hydrochloride (8.54 g, 122.3 mmol, 1.8 eq.) was added at once, affording a red solution which was stirred at 40° C. for 1.5 h. During this time the formed oxime started to precipitate as a bright yellow solid. The reaction mixture was cooled down to 0° C. for 1 h, affording an additional yellow precipitate. The solid was filtered, washed with EtOH (10 mL) and dried under reduced pressure to give the hydrochloride salt of the oxime as a bright yellow solid (5, 14 g, 41.82 mmol, 61%). HRMS found: [M+H]⁺ 299.1166. calcd for C₂₀H₁₅N₂O: 299.1179. ¹H NMR (400 MHz, methanol-d₄): δ 9.12 (1H, t, J=4.7 Hz), 8.63 (1H, s), 8.17 (1H, s), 7.99 (1H, t, J=4.6 Hz), 7.95 (1H, d, J=9.2 Hz), 7.82 (2H, t, J=4.6 Hz), 7.77 (1H, d, J=9.2 Hz), 7.57-7.50 (5H, m). ¹³C{¹H} NMR (100 MHz, methanol-d₄): δ 155.3, 147.5, 144.1, 140.25, 136.38, 134.5, 130.5, 130.2, 129.7, 129.5, 129.3, 128.8, 128.7, 128.3, 125.6, 125.45, 123.4, 122.2, 119.17.

When the 20 g (0.07 mol) of pure solid 4-phenylbenzoquinoline-2-carboxaldehyde (4) obtained in example 4 were slurried in 250 mL of ethanol followed by addition of 6.9 g (0.1 mol) of hydroxylamine hydrochloride in one lot, a quantitative yield of 20.93 g of 2-carbaldehyde-4-phenylbenzo[h]quinoline oxime hydrochloride 5 was obtained. The ethanol slurry of the 4-phenylbenzoquinoline-2-carboxaldehyde and the hydroxylamine hydrochloride was heated to 50° C. for 2 hours. The slurry was filtered and the solid product washed with ethanol.

### Example 5a

### Synthesis of 4-phenyl-2-cyanobenzo[h]quinoline (5a)

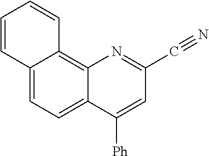 [SMILES: N#Cc1cc(-c2ccccc2)c2ccc3ccccc3c2n1]

10 g (0.03 mol) of 4-phenyl-2-bromo-benzo[h]quinoline was combined with 3.2 g (0.036 mol) of copper(I) cyanide and 50 mL of commercial grade N-methylpyrrolidone. The reaction mixture was heated to 150° C. for 4 hours, at which point no starting material remained. The cooled reaction mixture was quenched by its addition to a mixture of 10 g iron(III) chloride hexahydrate, 1 L of water and a few drops of concentrated hydrochloric acid. The mixture was extracted with dichloromethane. The dichloromethane phase was stripped and the crude product slurried in water to give a solid which was isolated by filtration. The crude product was taken up in toluene. Fractional crystallisation gives initially a compound fraction that was identified and characterised as 4-phenyl-benzo[h] quinoline-2-carboxamide (5b). As second pure fraction 3.3 g (39%) of the title compound 4-phenyl-2-cyanobenzo[h]quinoline 5a was isolated.

4-Phenyl-2-cyanobenzo[h] quinoline 5a: MS(ESI) m/z: 281 (MH+)¹H-NMR (DMSO-D6, 400 MHz) δ: 9.18 (1H, m), 8.18 (1H, s), 8.09 (2H, m, J=9.1), 7.86 (2H, m), 7.78 (1H, d, J=9.2), 7.64 (5H, m). ¹³C{¹H} NMR (DMSO-D6, 100 MHz) δ: 149.5, 146.7, 136.2, 133.3, 131.2, 131.1, 130.1, 129.9, 129.4, 129.0, 128.4, 128.3, 125.8, 125.7, 124.5, 122.2, 118.1 (shift overlap of two ¹³C resonances)

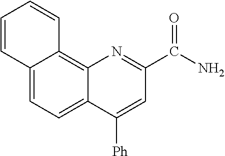 [SMILES: NC(=O)c1cc(-c2ccccc2)c2ccc3ccccc3c2n1]

4-Phenylbenzo[h]quinoline-2-carboxamide 5b: MS(ESI) m/z: 299 (MH+). ¹H-NMR (DMSO-D6, 400 MHz) δ: 9.68 (1H, m), 8.80 (1H, s), 8.19 (1H, s), 8.08 (1H, m), 8.03 (1H, d, J=9.2 Hz), 7.91 (1H, s), 7.84 (2H, m), 7.81 (1H, d, J=9.2 Hz), 7.63 (5H, m). ¹³C{¹H} NMR (DMSO-D6, 100 MHz) δ: 166.3, 149.2, 148.5, 145.2, 137.6, 133.3, 131.1, 129.7, 129.5, 129.2, 129.0, 128.9, 128.0, 127.7, 125.7, 125.1, 122.5, 120.0.

### Example 6

### Synthesis of 4-phenyl-2-aminomethyl-benzo[h]quinoline hydrochloride (HCNN^{Ph}.HCl) (6)

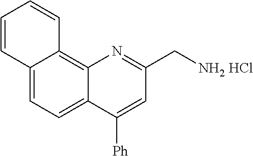

2-carbaldehyde-4-phenylbenzo[h]quinoline oxime hydrochloride (5, 6.0 g, 17.9 mmol) was placed in a 100 mL Parr autoclave followed by 10% Pd/C Type 338 (1.94 g of paste catalyst, Manufacturer Johnson Matthey). The autoclave was assembled, purged with nitrogen and depressurized. MeOH (60 mL) was added via the injection port. Stirring was started and autoclave was purged again with N₂ (5×2 bar) and H₂ (5×5 bar). The autoclave was pressurized with hydrogen to 5 bar and heated at 30° C. The gas uptake starts occurring after ca 45 min. Hydrogen was refilled to keep 5 bar and the reaction mixture was stirred until gas uptake was no longer apparent (ca. 90 min.). The autoclave was carefully depressurized and purged with N₂ (5×2 bar). Reaction mixture was filtered over a pad of celite and the pad was washed with MeOH (50 mL). The solvent was evaporated under reduced pressure to give the title compound as off-white solid (6, 5.5 g, 96% yield). HRMS found: [M+H]⁺ 285.1387. calcd for C₂₀H₁₇N₂: 285.1386. ¹H NMR (400 MHz, methanol-d₄): δ 9.52 (1H, d, J=8.0), 7.94 (1H, d, J=7.6), 7.86-7.70 (4H, m), 7.61-7.51 (6H, m), 4.5 (2H, s). ¹³C{¹H} NMR (100 MHz, methanol-d₄): δ 150.7, 149.9, 145.9, 137.8, 133.7, 131.0, 129.3, 128.5, 127.8, 127.5, 126.9, 124.8, 123.6, 122.2, 120.2, 43.1.

### Example 7

### Synthesis of N-(naphthalen-1-yl)-3-oxobutanamide (7)

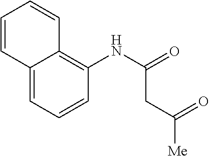 [SMILES: CC(=O)CC(=O)Nc1cccc2ccccc12]

The 1-naphthylamine reagent used might have contained a few ppm quantity of the highly carcinogenic 2-naphtylamine. While the 1-naphthylamine reagent had a quality allowing its use, 2-naphtylamine is banned from use in Europe and many other countries. An occupational health assessment required that in order to minimise exposure the N-(naphthalen-1-yl)-3-oxobutanamide 7 should be assayed and characterised as a crude product and then converted on as described in Example 8.

1-Naphthylamine (500 g, 3.49 mol) was placed in a 10 L round bottom flask and dissolved in THF (850 mL). Solid anhydrous sodium acetate (286.7 g, 3.6 mol) was charged next, followed by 2,2,6-trimethyl-4H-1,3-dioxin-4-one (700 g, 4.92 mol). The slurry was heated at reflux temperature for 26 hours. Then the reaction mixture was cooled to room temperature and 3 L dilute 2M aqueous HCl was added with vigorous stirring. The resulting slurry was stirred for 1 hour and then filtered. The pale purple solid was washed with water (2×150 mL) and dried in a desiccator over KOH at 40° C. 720 g of N-(naphthalen-1-yl)-3-oxobutanamide (7, 91% yield) were obtained. ¹H NMR (400 MHz, CDCl₃): δ 9.97 (br s, 1H, NH), 7.98 (d, 1H, J=7.5), 7.94 (d, 1H, J=8.3), 7.78 (d, 1H, J=8.0), 7.59 (d, 1H, J=8.3), 7.48 (t, 1H, J=7.5), 7.42 (t, 1H, J=7.6), 7.37 (t, 1H, J=8.0), 3.64 (s, 2H), 2.28 (s, 3H). ¹³C{¹H} NMR (100.61 MHz, CDC₃): δ 206.22 (C C═O), 163.9 (C C═O amide), 134.07, 132.28, 128.70, 126.60, 126.47, 125.74, 125.51, 120.76, 119.79, 49.19, 31.43.

In a repeat synthesis 1-Naphthylamine (900 g, 6.29 mol) was dissolved in 1.5 L of THF in a 10 L round bottom flask with overhead stirrer. Solid anhydrous sodium acetate (516 g, 6.29 mol) was charged next, followed by 2,2,6-trimethyl-4H-1,3-dioxin-4-one (1260 g, 8.86 mol). In this repeat 952 g of N-(naphthalen-1-yl)-3-oxobutanamide (7, 67% yield) were obtained.

### Example 8

### Synthesis of 4-methylbenzo[h]quinolin-2(1H)-one (8)

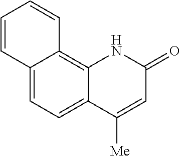 [SMILES: Cc1cc(=O)[nH]c2c1ccc1ccccc12]

3000 g of 98% sulphuric acid were heated in a 10 L round bottom flask with efficient overhead stirring to an internal temperature of 65° C. 952 g of N-(naphthalen-1-yl)-3-oxobutanamide 7 was added in portions so that despite the very exothermic reaction the internal temperature did not exceed 90° C. The mixture was heated to 95° C. for 1 hour and then cooled to 50° C. This mixture was slowly added to 15 kg of crushed ice in another 20 L round bottom flask with efficient overhead stirring. The slurry was stirred for 1 hour and then filtered. The purple solid was washed with 3×1 L of water and sucked dry as much as possible. The product is then transferred to a 10 L flask and stirred with 4 L of ethanol for 30 minutes. The slurry is again filtered and the pale purple solid is dried in a desiccator over KOH at 40° C. to afford the solid product (8, 814 g, 93% yield). HRMS found: [M+H]⁺ 210.0906. calcd for C₁₄H₁₀NO: 210.0913. ¹H NMR (400 MHz, CDCl₃): δ 8.51 (1H, d, J=8.3 Hz), 7.85 (1H, d, J=8.0 Hz), 7.77-7.56 (4H, m), 6.68 (1H, s), 2.56 (3H, s). ¹³C{¹H} NMR (100.61 MHz, CDCl₃): δ 162.84, 150.70, 134.71, 134.05, 128.73, 128.18, 127.28, 123.4, 121.29, 121.89, 121.03, 119.90, 116.96, 19.94.

### Example 9

### Synthesis of 2-bromo-4-methylbenzo[h]quinoline (9)

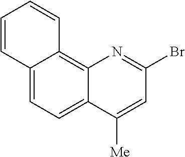 [SMILES: Cc1cc(Br)nc2c1ccc1ccccc12]

814 g of 4-methylbenzo[h]quinolin-2(1H)-one (8, 3.876 mol), obtained in Example 8 was dissolved in 4 L of toluene and azeotropically dried using Dean-Stark distillation. At room temperature 1115 g of POBr₃ was carefully added in portions. After the reaction mixture was heated overnight at 120° C. and 6.5 L of water were added, NaOH was added until a pH=14 was measured in the water phase. At this stage the reaction mixture had to be filtered over Celite to remove a very fine, very insoluble impurity. The Celite pad was washed with five 1 L quantities of toluene. The combined organic filtrate was dried and all toluene was removed by reduced pressure distillation. The residue was taken up in acetone (8.5 L), heated to reflux and the hot solution filtered through Celite. The acetone was partially distilled under reduced pressure until a very thick slurry was obtained. The slurry at room temperature was filtered to give the product 9 as a grey solid (584 g, 55% yield).

By repeating this reaction, another 621 g were obtained in higher yield of 75%. The 584 g and the 621 g were combined and dissolved in hot toluene, treated with activated charcoal and the charcoal was removed by filtration and the charcoal pad washed with further toluene. The combined toluene fractions were partially stripped giving a crop of 887 g of a pure cream solid 2-bromo-4-methylbenzo[h]quinoline 9. The water assay by Karl Fischer method gave 0.06% wt/wt residual water.

As further fractions 155 g, then 80 g of less pure material were obtained. Analysis on the pure product: HRMS found: [M+H]⁺ 272.0059. calcd. for C₁₄H₁₁BrN: 272.0069. ¹H NMR (400 MHz, CDCl₃): δ 9.0964 (1H, dd, J=2.0, 7.0 Hz), 7.75 (1H, dd, J=2.0, 7.0 Hz), 7.67 (s, 1H), 7.65 (s, 1H), 7.59 (1H, d, J=6.0 Hz), 7.65-7.56 (m, 1H), 7.31 (s, 1H), 2.51 (s, 3H). ¹³C{¹H} NMR (100 MHz, CDCl₃): δ 146.9, 146.5, 140.7, 133.5, 130.7, 128.5, 127.7, 127.6, 127.2, 126.6, 125.1, 124.6, 120.8, 18.7.

### Example 10

### Synthesis of 4-methylbenzo[h]quinoline-2-carbaldehyde (10)

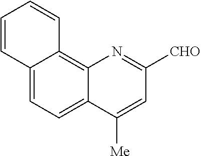 [SMILES: Cc1cc(C=O)nc2c1ccc1ccccc12]

54.4 g of 2-Bromo-4-methylbenzo[h]quinoline 9 (0.2 mol) was dissolved in 400 mL of THF in a 1 L three neck round bottom flask and the mixture was cooled to −75° C. (IPA/dry ice bath). 100 mL of 2.5 m n-butyl lithium in hexanes (0.25 mol, 1.25 eq) was added slowly so that the internal temperature never went above −70° C. The mixture was left to stir for 45 minutes at −75° C. Then 22 g of anhydrous dimethylformamide (0.30 mol, 1.5 eq) was added in small drops so that the internal temperature never went above −65° C. The reaction was then allowed to reach room temperature overnight. The next day, 350 mL of water was added to quench the reaction, followed by 40 mL of glacial acetic acid. A solid precipitated and was filtered off and washed with water and n-heptane to give a first crop. The organic layer of the filtrate was separated from the aqueous phase and the solvents were removed by distillation at reduced pressure. The residue was triturated with 150 mL of methanol to give a second crop that was filtered off and washed with methanol. The two crops were combined and dried under vacuum affording compound 10. Yield 29.4 g (68%). ¹H NMR (400 MHz, CDCl₃): δ 10.35 (1H, s), 9.47 (1H, d, J=8.1 Hz), 8.04-7.93 (4H, m), 7.86-7.75 (3H, m), 2.89 (3H, s). ¹³C{¹H} NMR (100 MHz, CDCl₃): δ 194.5, 150.5, 146.1, 145.4, 133.4, 131.9, 130.3, 128.7, 128.4, 127.9, 127.7, 121.1, 119.3, 19.3.

### Example 11

### Synthesis of 4-methylbenzo[h]quinoline-2-carbaldehyde oxime hydrochloride (11)

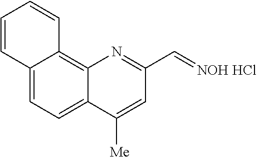

29 g of 4-methylbenzo[h]quinoline-2-carbaldehyde 10 (0.13 mol) from example 10 were slurried in 300 mL of ethanol followed by addition of 9.6 g (0.143 mol) of hydroxylamine hydrochloride in one lot. The ethanol slurry of the 4-phenylbenzoquinoline-2-carboxaldehyde and the hydroxylamine hydrochloride was heated to 50° C. for 90 minutes and the slurry then filtered and the solid product washed with cold ethanol. A 76% yield of 26.0 g of 2-carbaldehyde-4-methylbenzo[h]quinoline oxime hydrochloride 11 was obtained. HRMS found: [M+H]⁺ 237.1018. calcd for C₁₅H₁₃N₂O: 237.1022. ¹H NMR (400 MHz, methanol-d₄): δ 9.16-9.10 (1H, m), 8.75 (1H, s), 8.32 (1H, s), 8.20-8.10 (3H, m), 7.95-7.91 (2H, m), 3.05 (3H, s). ¹³C{¹H} NMR (100 MHz, methanol-d₄): δ 155.6, 146.6, 142.7, 137.4, 134.7, 130.79, 130.4, 129.0, 128.5, 127.2, 124.1, 123.03, 120.7, 119.7, 19.3.

### Example 11a

### Synthesis of 4-methyl-2-cyanobenzo[h]quinoline (11a)

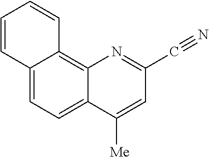 [SMILES: Cc1cc(C#N)nc2c1ccc1ccccc12]

13.6 g (0.035 mol) of 4-methyl-2-bromo-benzo[h]quinoline (9) were combined with 5.6 g (0.062 mol) of copper(I) cyanide and 75 mL of commercial grade N-methylpyrrolidone. The reaction mixture was heated to 150° C. for 4 hours, at which point no starting material remained. The cooled reaction mixture was quenched by its addition to a mixture of 20 g iron(III) chloride hexahydrate, 150 mL of water and 2 mL of concentrated hydrochloric acid. A solid precipitated and was isolated by filtration. After washing with water and drying the solid was recrystallized from toluene to give 7.5 g of 4-methyl-2-cyanobenzo[h]quinoline (11a) contaminated with less than 20% w/w 4-methyl-benzo[h]quinoline-2-carboxamide (11b).

4-methyl-2-cyanobenzo[h]quinoline 11a: MS(ESI) m/z: 219 (MH⁺). ¹H-NMR (DMSO-D6, 400 MHz) δ: 9.08 (1H, m), 8.13-7.96 (4H, m), 7.82 (2H, m), 2.75 (3H, s). ¹³C{¹H} NMR (DMSO-D6, 100 MHz) δ: 147.3, 133.4, 130.6, 129.5, 128.3, 128.2, 126.9, 126.3, 124.4, 121.5, 118.2, 18.6 (shift overlap of four ¹³C resonances).

4-Methylbenzo[h]quinoline-2-carboxamide 11b has been identified as a separate peak in LCMS with MS(ESI) m/z: 237 (MH+). Mass difference+18 (water) as expected.

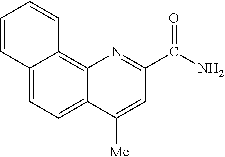 [SMILES: Cc1cc(C(N)=O)nc2c1ccc1ccccc12]

### Example 12

### Synthesis of 4-methyl-2-aminomethyl-benzo[h]quinoline hydrochloride (HCNN^{Me}.HCl) (12)

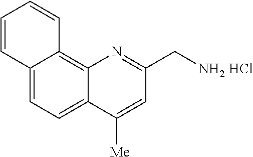

Compound 11 (100 mg, 0.36 mmol) was placed in an glass insert of an Biotage Endeavor pressure screening unit followed by 10% Pd/C Type 338 (15 mg of paste catalyst, Johnson Matthey product). The Biotage Endeavor was assembled, the vial purged with nitrogen and depressurized. MeOH (3 mL) was added via the injection port. The stirring was started and the autoclave was purged with N₂ (5× to 2 bar) and H₂ (5× to 5 bar). The system was pressurized with hydrogen to 5 bar and heated at 30° C. Gas uptake started after 45 minutes. The hydrogen pressure was kept at 5 bar and the reaction mixture was stirred until gas uptake was no longer apparent (ca. 90 min). The system was carefully depressurized and purged with N₂ (5× to 2 bar). The reaction mixture was filtered over a pad of celite and the pad was washed with MeOH (10 mL). The solvent was evaporated under reduced pressure to give 4-methyl-2-aminomethyl-benzo[h]quinoline hydrochloride 12 as an off-white solid (93 mg). HRMS found: [M+H]⁺ 223.1230. calcd for C₁₅H₁₅N₂: 223.1230. ¹H NMR (400 MHz, methanol-d₄): δ 7.89 (1H, d, J=8.0 Hz), 6.41 (2H, d, J=8.7 Hz), 6.35 (1H, d, J=9.1 Hz), 6.25-6.14 (2H, m), 5.92 (1H, s), 2.97 (2H, s), 1.21 (3H, s). ¹³C{¹H} NMR (100 MHz, methanol-d₄): δ 149.0, 144.5, 143.4, 132.2, 129.6, 126.5, 126.0, 125.9, 125.2, 123.6, 123.2, 119.3, 119.0, 41.4, 16.2.

### Example 13

### Synthesis of RuCl(CNN^{Ph})(dppp) (13)

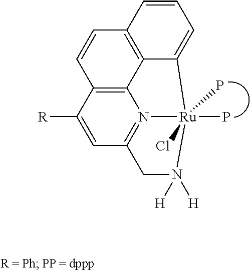

RuCl₂(PPh₃)₃ (222 mg, 0.232 mmol) and dppp (101 mg, 0.244 mmol) were slurried in 2-propanol (4 mL) and the mixture was refluxed in a 25 mL round bottom flask for 1 h. Compound 6 (82 mg, 0.256 mmol) and NEt₃ (0.32 mL, 2.3 mmol) were added and the mixture was refluxed for 1 h. The suspension was cooled to room temperature and heptane (4 mL) was added. The orange precipitate was filtered, washed with MeOH (1 mL), heptane (3×1 mL) and dried under reduced pressure (13, 171 mg, 89% yield). HRMS found: [M-Cl]⁺797.1787. calcd for C₄₇H₄₁N₂P₂Ru: 797.1783. ¹H NMR (200.1 MHz, CD₂Cl₂): δ 8.06 (m, 1H), 7.95-7.81 (m, 4H), 7.66-6.90 (m, 21H), 6.59 (t, J=7.1 Hz, 1H), 6.28 (t, J=6.8 Hz, 2H), 5.77 (t, J=8.2 Hz, 2H), 4.53 (d, J=14.4 Hz, NCH₂, 1H), 4.12 (m, NH₂, 1H), 3.91 (m, NCH₂, 1H), 2.98 (t, J=12.6 Hz, CH₂, 1H), 2.65 (t, J=12.4.1 Hz, CH₂, 1H), 2.37 (t, J=13.8 Hz, CH₂, 1H), 2.20 (m, NH₂, 1H), 1.72-1.58 (m, CH₂, 2H), 1.29 (m, CH₂, 1H). ¹³C{¹H} NMR (50.3 MHz, CD₂Cl₂): δ 170.0 (dd, ²J(CP)=15.1, 9.5 Hz; CRu), 155.2, 152.2, 146.4, 146.1, 143.2, 142.4, 138.9, 138.6, 138.1, 137.7, 137.1, 135.9, 135.7, 134.3, 133.8, 133.6, 133.4, 131.6, 131.5, 130.0, 129.8, 129.2, 129.1, 128.9, 128.8, 128.6, 128.5, 128.2, 128.0, 127.6, 127.4, 127.2, 125.9, 125.7, 123.0, 120.5, 118.4, 117.1, 52.0 (d, J=2.0 Hz, CH₂N) 29.8, (d, J=26.4 Hz, CH₂P), 24.8, (d, J=35.6 Hz, CH₂P), 21.0 (s, CH₂P). ³¹P{¹H} NMR (81.0 MHz, CD₂Cl₂): δ 54.7 (d, J=48.6 Hz), 35.7 (d, J=48.6 Hz).

### Example 14

### Synthesis of RuCl(CNN^{Ph})(dppb) (14)

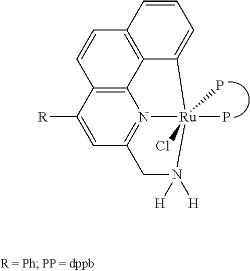

RuCl₂(PPh₃)₃ (2.22 g, 2.32 mmol) and dppb (1.04 g, 2.44 mmol) were suspended in anhydrous 2-propanol (40 mL) and the mixture was refluxed in a 250 mL round bottom flask for 1.5 h. Compound 6 (820 mg, 2.56 mmol) and NEt₃ (3.2 mL, 23 mmol) were added and the mixture was refluxed for 1.5 h. The suspension was cooled to room temperature and the bright orange precipitate was filtered, washed with MeOH (10 mL), heptane (3×10 mL) and dried under reduced pressure (14, 1.68 g, 85% yield). HRMS found: [M-Cl]⁺811.1943. calcd for C₄₈H₄₃N₂P₂Ru 811.1940. ¹H NMR (200.1 MHz, CD₂Cl₂): δ 8.25 (pseudo t, J=7.6 Hz, 2H, aromatic protons), 8.04 (d, J=7.0 Hz, 1H, aromatic proton), 7.85 (pseudo t, J=8.0 Hz, 2H, aromatic protons), 7.65-7.31 (m, 20H, aromatic protons), 6.95 (s, 1H, aromatic proton), 6.56 (t, J=7.2 Hz, 1H, aromatic proton), 6.23 (pseudo t, J=7.4 Hz, 2H, aromatic protons), 5.54 (t, J=7.8 Hz, 2H, aromatic protons), 4.37 (dd, J=16.2, 5.2 Hz, 1H, NCH₂), 4.02 (m, 1H, NCH₂), 3.68 (m, 1H, NH₂), 2.96 (m, 2H, CH₂), 2.38-1.00 (m, 7H, CH₂ and NH₂). ¹³C{¹H} NMR (50.3 MHz, CD₂Cl₂): δ 168.1, 166.0, 159.3, 159.1, 157.5, 150.0, 149.8, 144.8, 144.7, 143.3, 142.6, 142.2, 142.1, 141.8, 141.6, 141.4, 141.3, 140.8, 140.6, 139.1, 139.0, 133.9, 109.6, 43.9, 43.2, 40.1, 35.2. ³¹P{¹H} NMR (81.0 MHz, CD₂Cl₂): δ 57.3 (d, J=38.1 Hz), 43.3 (d, J=38.1).

### Example 15

### Synthesis of RuCl(CNN^{Ph})(dppf) (15)

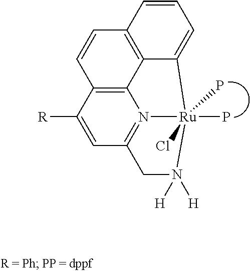

RuCl₂(PPh₃)₃ (2.22 g, 2.32 mmol) and dppf (1.54 mg, 2.78 mmol) were suspended in 2-propanol (20 mL) and the mixture was refluxed in a 250 mL round bottom flask for 1.5 h. Compound 6 (820 mg, 2.55 mmol) and NEt₃ (3.2 mL, 23 mmol) were added and the mixture was refluxed for 5 h. The suspension was cooled to room temperature and heptane (40 mL) was added. The orange precipitate was filtered, washed with MeOH (10 mL), heptane (3×10 mL) and dried under reduced pressure (15, 2.03 g, 90% yield). HRMS found: [M-Cl]⁺939.1301. calcd for C₅₄H₄₃FeN₂P₂Ru: 939.1289. ¹H NMR (200.1 MHz, CD₂Cl₂): δ 8.65-8.5 (m, 1H), 8.13 (pseudo t, J=7.6 Hz, 2H), 7.85-7.0 (m, 22H), 6.64 (pseudo t, J=7.4 Hz, 1H), 6.31 (pseudo t, J=7.2 Hz, 2H), 6.08 (pseudo t, J=8.0 Hz, 2H), 4.85 (m, 1H), 4.5-4.1 (m, 5H), 3.9-3.68 (m, 3H), 3.2 (m, 1H), 2.24 (m, 1H). ¹³C{¹H} NMR (50.3 MHz, CD₂Cl₂): δ 176.3, 173.8, 167.9, 167.6, 167.3, 165.2, 164.6, 160.9, 160.2, 159.4, 159.1, 158.9, 155.3, 155.1, 154.9, 154.1, 153.3, 153.1, 151.6, 150.9, 150.7, 150.5, 150.2, 149.7, 149.5, 149.3, 149.1, 148.9, 147.9, 147.6, 147.4, 146.7, 146.6, 143.5, 141.4, 139.9, 138.4, 109.0, 108.2, 107.7, 106.7, 98.8, 98.5, 98.1, 96.8, 95.1, 94.6, 90.5, 90.4, 90.1, 53.1, 50.3, 44.0, 35.1. ³¹P{¹H} NMR (81.0 MHz, CD₂Cl₂): δ 62.0 (d, J=35.6 Hz), 45.3 (d, J=35.6 Hz).

### Example 16

### Synthesis of RuCl(CNN^{Me})(dppp) (16)

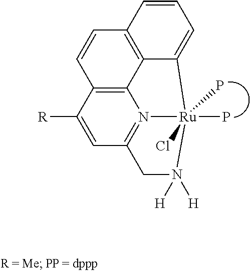

In a 100 mL Schlenk were introduced, under argon atmosphere, RuCl₂(PPh₃)₃ (445 mg, 0.46 mmol), dppp (199 mg, 0.48 mmol) and 2-propanol (10 mL). The reaction mixture was refluxed for 1 h, compound 12 (131 mg, 0.506 mmol) and triethylamine (0.64 mL, 4.6 mmol) were added and the reaction mixture was refluxed overnight. The reaction mixture was cooled to RT and the solid was filtered off. The precipitate was washed with MeOH (2 mL) and dried in vacuum (16, 278 mg, 78% yield). HRMS found: [M-Cl]⁺735.1638. calcd for C₄₂H₃₉N₂P₂Ru: 735.1627. ¹H NMR (400 MHz, CDCl₃): δ 8.47 (pseudo t, J=8.0 Hz, 2H), 8.37 (d, J=6.5 Hz, 1H), 8.11 (pseudo t, J=8.0 Hz, 2H), 8.0-6.9 (m, 15H), 6.48 (t, J=7.2 Hz, 1H), 6.42 (s, 1H, aromatic proton), 6.22 (t, J=6.9 Hz, 2H), 5.85 (t, J=8.1 Hz, 2H), 4.56 (m, 1H), 3.79 (m, 2H), 3.25 (m, 1H), 3.08 (m, 2H), 2.83 (m, 1H), 2.62 (m, 1H), 2.30 (m, 5H). ¹³C{¹H} NMR (100 MHz, C₆D₆): 142.9, 139.9, 139.4, 139.0, 136.2, 133.3, 132.1, 128.4, 127.1, 126.7, 125.2, 64.5, 28.5, 25.2, 18.7, 17.8. ³¹P{¹H} NMR (162 MHz, C₆D₆): δ 54.2 (d, J=47.7 Hz), 35.5 (d, J=47.7 Hz).

### Example 17

### Synthesis of RuCl(CNN^{Me})(dppb) (17)

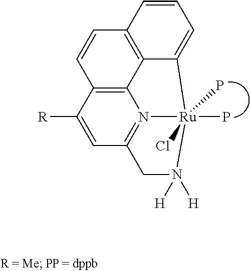

The preparation of 17 was carried out substantially as described for complex 16, but using dppb (237 mg, 0.56 mmol) in place of dppp (17, 300 mg, 83% yield). HRMS found: [M-Cl]⁺749.1788. calcd for C₄₃H₄₁N₂P₂Ru: 749.1783. ¹H NMR (400 MHz, CDCl₃): δ 8.71 (pseudo t, J=8.0 Hz, 2H), 8.45 (d, J=7.0 Hz), 8.23 (pseudo t, J=8.0 Hz, 2H), 7.85-7.36 (m, 8H), 7.25-7.16 (m, 7H), 6.39 (pseudo t, J=7.4 Hz, 1H), 6.28 (s, 1H, aromatic proton), 6.15 (pseudo t, J=7.5 Hz, 2H), 5.67 (t, J=7.8 Hz, 2H), 4.09 (m, 1H), 3.55-3.4 (m, 2H), 3.25-3.15 (m, 2H), 2.4-2.3 (m, 1H), 2.15 (s, 3H, Me), 2.10-1.70 (m, 6H). ¹³C{¹H} NMR (100 MHz, C₆D₆): 155.1, 153.7, 146.4, 154.3, 144.6, 141.7, 136.4, 136.3, 133.8, 133.6, 131.4, 131.3, 131.2, 130.6, 130.5, 129.6, 129.1, 125.8, 125.1, 125.0, 123.8, 118.6, 116.7, 51.6, 33.0, 29.7, 26.3, 21.4, 17.8. ³¹P{¹H} NMR (162 MHz, CDCl₃): δ 57.5 (d, J=38.5 Hz), 43.2 (d, J=38.5 Hz).

### Example 18

### Synthesis of RuCl(CNN^{Me})(dppf) (18)

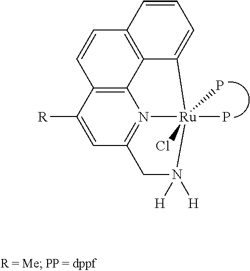

The preparation of 18 was carried out substantially as described for complex 16, but using dppf (311 mg, 0.56 mmol) in place of dppp (18, 184 mg, 44% yield). HRMS found: [M-Cl]⁺877.1148. calcd for C₄₉H₄₁FeN₂P₂Ru: 877.1132. ¹H NMR (400 MHz, CDCl₃): δ 9.11 (d, J=6.0 Hz, 1H), 9.0 (pseudo t, J=8.0 Hz, 2H), 8.79 (m, 1H), 8.0-7.71 (m, 5H), 7.4-7.07 (m, 10H), 6.45 (t, J=6.4 Hz, 1H), 6.27-6.17 (m, 4H), 5.8 (s, 1H, aromatic proton), 5.01 (m, 1H), 4.38-4.35 (m, 2H), 4.2 (m, 1H), 3.98 (m, 2H), 3.65 (m, 2H), 3.32 (m, 1H), 3.08 (m, 1H), 2.22 (s, 3H, Me), 1.92 (m, 1H). ¹³C{¹H} NMR (100 MHz, C₆D₆): 176.8, 155.4, 152.1, 147.2, 146.8, 144.1, 143.8, 141.6, 139.7, 139.4, 138.7, 138.6, 135.8, 135.4, 135.2, 133.9, 132.6, 126.6, 126.1, 125.3, 123.8, 119.0, 118.6, 117.5, 88.7, 88.3, 86.8, 86.3, 77.9, 77.2, 75.5, 73.9, 73.3, 69.2, 68.9, 68.6, 63.6, 50.9, 25.2, 17.9. ³¹P{¹H} NMR (162 MHz, C₆D₆): δ 61.4 (d, J=35.7 Hz), 45.1 (d, J=35.7 Hz).

### Example 19

### Synthesis of RuCl(CNN^{Ph})(rac-BINAP) (19)

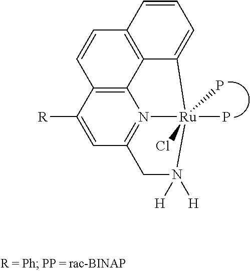

[RuCl₂(p-cymene)]₂ (71 mg, 0.116 mmol) and rac-BINAP (152 mg, 0.244 mmol) were suspended in 2-propanol (2 mL) and the mixture was refluxed in a 25 mL round bottom flask for 2 h. Compound 6 (82 mg, 0.256 mmol) and NEt₃ (0.32 mL, 2.3 mmol) were added and the mixture was refluxed for 6 h. The mixture was cooled to room temperature and heptane (4 mL) was added. The precipitate was filtered, washed with MeOH (1 mL), diethyl ether (5×2 mL) and dried under reduced pressure, obtaining the complex as mixture of two stereoisomers in about 4/3 molar ratio (19, 150 g, 62% yield). HRMS found: [M-Cl]⁺1007.2254. calcd for C₆₄H₄₇N₂P₂Ru: 1007.2258. ¹H NMR (200.1 MHz, CD₂Cl₂): δ 8.59 (d, J=6.4 Hz), 8.40-8.13 (m), 8.02-6.22 (m, aromatic protons), 6.13-5.78 (m, aromatic protons), 5.36 (d, J=7.6 Hz), 4.74-3.35 (m, CH₂ and NH₂), 2.43-2.24 (m, CH₂), 1.73-1.40 (m, NH₂). ¹³C{¹H} NMR (50.3 MHz, CD₂Cl₂): δ 178.0 (dd, J=12.3, 9.1 Hz, CRu), 176.5 (dd, J=14.3, 9.2 Hz, CRu), 156.3, 154.6, 153.6, 153.2, 147.5-123.4 (m, aromatic carbon atoms), 120.5, 120.0, 119.3, 118.2, 117.4 (d, J=2.6 Hz), 115.9 (d, J=2.7 Hz), 52.8 (br s, NCH₂), 52.4 (br s, NCH₂). ³¹P{¹H} NMR (81.0 MHz, CD₂Cl₂): δ 60.6 (minor diastereoisomer, d, J=39.7 Hz), 52.4 (minor diastereoisomer, d, J=39.7 Hz), 52.1 (major diastereoisomer, d, J=34.8 Hz), 51.2 (major diastereoisomer, d, J=34.8 Hz).

### Example 20

### Synthesis of RuCl(CNN^{Ph})[(S,R)-JOSIPHOS)] (20)

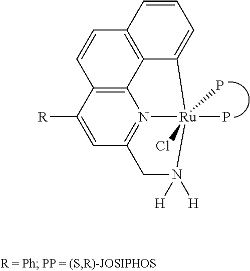

[RuCl₂(p-cymene)]₂ (71.0 mg, 0.116 mmol) and (S,R)-JOSIPHOS (165.5 mg, 0.278 mmol) were suspended in 2-propanol (4 mL) and the mixture was refluxed in a 25 mL round bottom flask for 1 h. Compound 6 (82 mg, 0.256 mmol) and NEt₃ (0.32 mL, 2.3 mmol) were added and the mixture was refluxed for 5 h. The solvent was removed and the solid was dried under reduced pressure The solid was dissolved in CH₂Cl₂ (1 mL), kept at −20° C. for 18 h, affording the precipitation of triethylammonium chloride which was eliminated by filtration. Addition of heptane (2 mL) to the filtrate gave an orange precipitate which was filtered, washed with heptane and dried under reduced pressure (20, 125 mg, 53% yield). HRMS found: [M-Cl]⁺979.2543. calcd for C₅₆H₅₉FeN₂P₂Ru: 979.2546. ¹H NMR (200.1 MHz, CD₂Cl₂): δ 8.38 (d, J=7 Hz, 1H), 8.21 (m, 2H), 7.82-7.13 (m, 20H), 4.76-4.35 (m, 5H), 4.22 (m, 1H), 3.79 (s, 5H), 1.98-1.7 (m, 3H), 1.45-0.95 (m, 22H). ¹³C{¹H} NMR (50.3 MHz, CD₂Cl₂): δ 157.3, 154.8, 147.7, 146.6, 146.0, 145.2, 144.7, 144.6, 140.1, 139.2, 138.7, 137.3, 133.4, 132.2, 130.1, 129.8, 129.2, 128.9, 128.6, 127.5, 127.1, 126.6, 120.3, 118.2, 117.2, 97.6 (dd, J=21.2 Hz, J=3.1 Hz; ipso-O₅H₃), 74.0 (s; C₅H₃), 72.5 (dd, J=37.2 Hz, J=5.0 Hz ipso-O₅H₃), 70.4 (s; C₅H₅), 69.8 (d, J=13.3 Hz; C₅H₃), 68.5 (m, C₅H₃), 52.2 (d, J=2.3 Hz; NCH₂), 40.0 (d, J=15.8 Hz; CH of Cy), 37.6 (d, J=17.6 Hz; CH of Cy), 31.5-26.2 (m; CH₂ of Cy), 29.1 (d, J=3.8 Hz; PCHCH₃), 15.5 ppm (d, J=6.9 Hz; PCHCH₃). ³¹P{¹H} NMR (81.0 MHz CD₂Cl₂): δ 66.5 (d, J=42.1 Hz), 41.3 (d, J=42.1 Hz).

### Example 21

### Synthesis of 1-napthyl-propionamide (21)

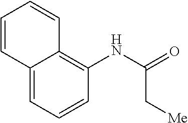 [SMILES: CCC(=O)Nc1cccc2ccccc12]

The 1-naphthylamine reagent used might have contained a few ppm quantity of the highly carcinogenic 2-naphtylamine. While the 1-naphthylamine reagent had a quality allowing its use, 2-naphtylamine is banned from use in Europe and many other countries. An occupational health assessment required that in order to minimise exposure the N-(naphthalen-1-yl)-propionamide 21 should be assayed and characterised as a crude product and then converted on as described in Example 22.

In a 500 mL round bottomed flask were introduced 1-naphthylamine (28.0 g; 156 mmol) and 200 mL of dry dichloromethane. The solution was cooled to 0° C. and triethylamine (24.0 mL, 172 mmol) was added. Propionyl chloride (15.88 g; 171.6 mmol; 14.8 mL) was slowly ((due to a very exothermic reaction) added. The reaction mixture was stirred at 0° C. and allowed to warm up slowly to room temperature. A formed precipitate was removed by filtration and the filtrate was extracted with 10% aqueous hydrochloric acid. The aqueous extract was further extracted twice with 100 mL of dichloromethane. The dichloromethane layers were combined and dried over magnesium sulfate. Dichloromethane was removed under reduced pressure, affording 21. Yield: 21.90 g; 109.9 mmol, 71%. ¹H NMR (400 MHz, CDCl₃): δ 7.70 (4H, t, J=7.4 Hz), 7.56 (1H, d, J=7.9 Hz), 7.37 (2H, m, broad), 7.30 (1H, t, J=7.6 Hz), 2.36 (2H, d, J=7.0 Hz), 1.17 (3H, t, J=7.0 Hz). ¹³C{¹H} NMR (100.61 MHz, CDC₃): δ 172.95, 134.09, 128.62, 127.20, 126.14, 125.91, 125.82, 125.63, 121.39, 120.96, 30.43, 9.94 (possible overlap of two carbon resonances).

### Example 22

### Synthesis of 2-chloro-3-methylbenzo[h] quinoline (22)

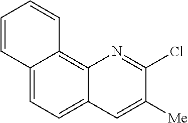 [SMILES: Cc1cc2ccc3ccccc3c2nc1Cl]

In a 100 mL two neck round bottom flask were introduced, under argon atmosphere, 1-naphthyl-propionamide (10.0 g, 50.2 mmol) and anhydrous dimethylformamide (3.89 mL, 1 eq). POCI₃ (20 mL; 4.2 eq) was added dropwise and the reaction mixture was heated to reflux releasing the formed HCl gas through a silicone oil filled bubbler. After heating overnight the reaction mixture was cooled to room temperature and then carefully hydrolysed in a mixture of crushed ice and water. After stirring for 2 hours, a precipitate had formed that was filtered off, washed with water and dried in vacuum. A yield of 8.01 g (35.18 mmol, 70%) of 22 was obtained. ¹H NMR (400 MHz, CDCl₃): δ 9.19 (d, 1H, J=7.9 Hz), 7.93 (s, 1H), 7.88 (d, 1H, J=7.9 Hz) 7.78 (d, 1H, J=8.8 Hz), 7.71 (q, 2H, J=7.3 Hz), 7.58 (d, 1H, J=8.8 Hz) 2.55 (s, 3H). ¹³C{¹H} NMR (100.61 MHz, CDC₃): δ 150.67, 144.66, 138.02, 133.40, 130.64, 130.42, 128.24, 128.00, 127.74, 127.15, 125.59, 124.43, 124.20, 19.96.

### Example 23

### Transfer Hydrogenation of Ketones.

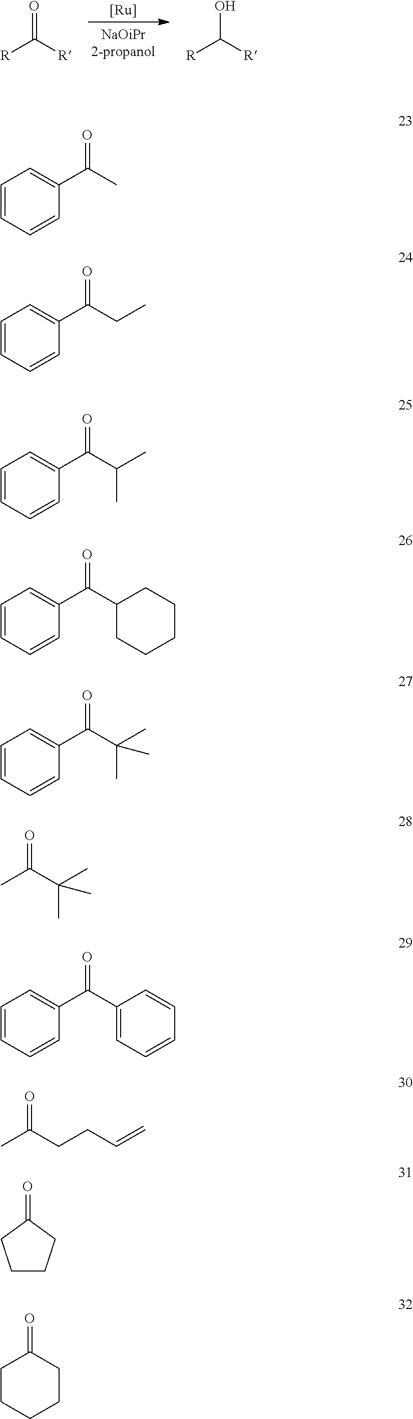

The catalyst (2.5 μmol) used was dissolved in 2.5 mL of 2-propanol. The ketone (2.0 mmol) was dissolved in 2-propanol and the solution (final volume 19.4 mL) was heated under argon at reflux. By addition of 400 μL of NaOiPr (0.1 M, 40 μmol) in 2-propanol and 200 μL of the solution containing the catalyst the reduction of the ketone started immediately and the yield was determined by GC after reaction times given in the Table 1

TABLE 1
Catalytic transfer hydrogenation of ketones (0.1M) with complexes 13-20
(S/C = 5000-20000) and NaOiPr (2 mol %) in 2-propanol at 82° C.

| | | | | | | Conv. |
|---|---|---|---|---|---|---|
| Entry | Complex | Ketone | S (M) | S/C | t (min) | (%)^{a} |
| 1 | 13 | 23 | 0.1 | 10000 | 2 | 98 |
| 2 | 14 | 23 | 0.1 | 5000 | 10 | 99 |
| 3 | 14 | 23 | 0.1 | 10000 | 15 | 99 |
| 4 | 14 | 23 | 0.1 | 20000 | 15 | 98 |
| 5 | 14 | 23 | 0.2 | 10000 | 15 | 96 |
| 6 | 14 | 23 | 0.5 | 10000 | 15 | 93 |
| 7 | 15 | 23 | 0.1 | 10000 | 2 | 97 |
| 8 | 15 | 23 | 0.1 | 20000 | 10 | 97 |
| 9 | 16 | 23 | 0.1 | 10000 | 10 | 96 |
| 10 | 17 | 23 | 0.1 | 10000 | 10 | 95 |
| 11 | 18 | 23 | 0.1 | 10000 | 20 | 93 |
| 12 | 19 | 23 | 0.1 | 10000 | 10 | 97 |
| 13 | 20 | 23 | 0.1 | 10000 | 2 | 97^{b} |
| 14 | 17 | 24 | 0.1 | 10000 | 10 | 94 |
| 15 | 15 | 25 | 0.1 | 10000 | 20 | 99 |
| 16 | 15 | 26 | 0.1 | 10000 | 40 | 95 |
| 17 | 18 | 26 | 0.1 | 10000 | 20 | 93 |
| 18 | 13 | 27 | 0.1 | 5000 | 40 | 99 |
| 19 | 18 | 27 | 0.1 | 5000 | 14 h | 97 |
| 20 | 13 | 28 | 0.1 | 5000 | 40 | 99 |
| 21 | 18 | 28 | 0.1 | 5000 | 14 h | 98 |
| 22 | 15 | 29 | 0.1 | 10000 | 10 | 99 |
| 23 | 15 | 30 | 0.1 | 10000 | 10 | 98 |
| 24 | 17 | 31 | 0.1 | 10000 | 5 | 99 |
| 25 | 17 | 32 | 0.1 | 10000 | 5 | 99 |

^{a}The conversion was determined by GC analysis
^{b}ee = 85% (S)

The catalysts of this investigation reduce a wide structural variety of ketones. In 2-propanol at reflux and in the presence of NaOiPr (2 mol %) the ketones in Table 1 are efficiently reduced via transfer hydrogenation with a S/C ratio up to 20000/1. The ketones are selected to cover a broad range of structures: alkyl-arylketones 23-27, benzophenone 29 and dialkylketones 28, 30-32. Ketones 27 and 28 having bulky ^{tert}Bu substituents are reduced with near complete conversion of the substrate. Reduction of C═O bond of 5-hexen-2-one 30 is entirely chemoselective, without saturation or isomerization of the terminal C═C bond.

The use of methyl-benzo[h]quinoline or phenyl-benzo[h] quinoline ligands allows a fine tuning of catalyst activity and selectivity. The chiral complex 20 containing the (S,R)-JOSIPHOS ligand reduced 23 quantitatively to (S)-1-phenylethanol in 2 min and with 85% ee.

### Example 24

### Diastereomeric Transfer-Hydrogenation of L-Menthone

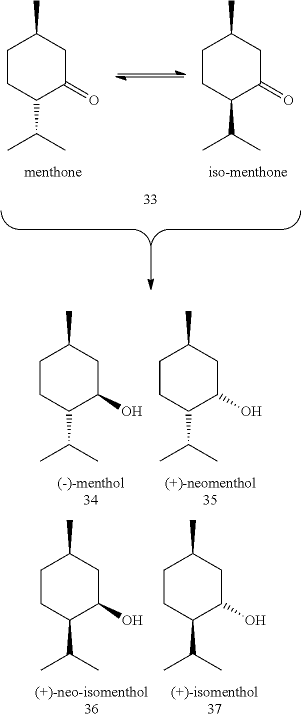

A single batch of L-menthone 33 (Alfa Aesar, Product A13679, batch 10171537) was used for this comparative example. In the presence of even traces of either acid or base the menthone diastereomer equilibrates with the isomenthone diastereomer.

Catalytic Runs were Carried Out (Table 2).

The complex (1 μmol) and 0.17 mL (1 mmol) of L-menthone were dissolved in 9.83 mL of 2-propanol and the solution was purged with argon with three vacuum/argon cycles. The mixture was then heated in an oil bath at reflux. After 2 minutes at temperature, 0.2 mL (0.02 mmol) of a 0.1M solution of NaOi-Pr in 2-propanol was added and the samples were analysed by GC after reaction times given in the Table 2. Pure samples of (−)-menthol 34, (+)-neomenthol 35, and (+)-isomenthol 37 were used as analytical standards to confirm the identity of GC peaks.

TABLE 2
Catalytic transfer hydrogenation of menthone (0.1M) with
complexes 13-18 (S/C = 1000) and NaOiPr (2 mol %) in 2-propanol.

| | | T | Time | Conv. | 34 | 35 | 36 | 37 |
|---|---|---|---|---|---|---|---|---|
| Entry | Complex | (° C.) | (h) | (%)^{a} | (%) | (%) | (%) | (%) |
| 1 | 13 | 82 | 3 | 98 | 2 | 79 | 4 | 13 |
| 2 | 14 | 82 | 1 | 97 | 2 | 58 | 25 | 12 |
| 3 | 15 | 82 | 2 | 90 | 14 | 48 | 18 | 10 |
| 4 | 16 | 50 | 1 | 94 | 6 | 57 | 20 | 11 |
| 5 | 17 | 82 | 1 | 92 | 4 | 53 | 23 | 12 |
| 6 | 18 | 82 | 4 | 99 | 72 | 18 | 6 | 3 |

^{a}The conversion was determined by GC analysis.

Catalysts 13-17 convert the substrate mainly to (+)-neomenthol 35 (derived from the menthone diastereomer) and to 36, 37 (both derived from the iso-menthone diastereomer). Surprisingly, the complex 18 is both selective in the formation of the (−)-menthol 34 and more selective than others in the substrate consumption, preferring reaction with the menthone diastereomer over the iso-menthone diastereomer.

### Example 25

Transfer Hydrogenation of α,β-unsaturated Ketones

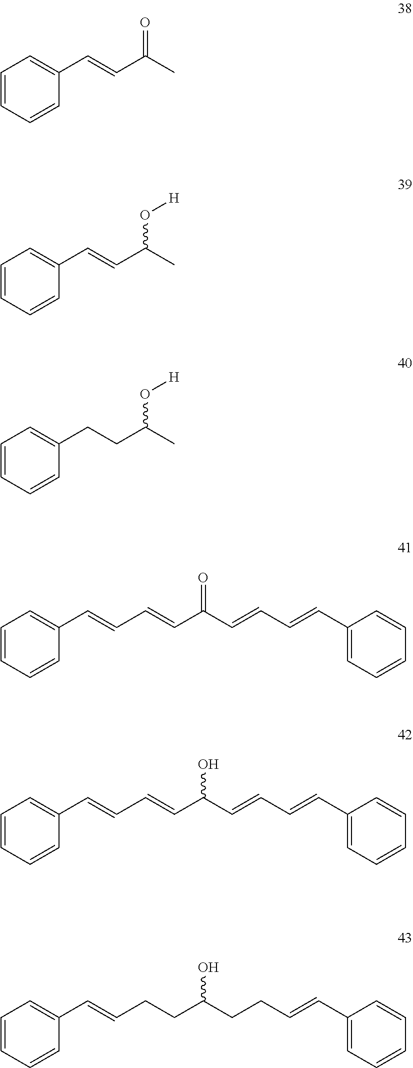 [SMILES: CC(=O)/C=C/c1ccccc1 ; CC(O)/C=C/c1ccccc1 ; CC(O)CCc1ccccc1 ; O=C(/C=C/C=C/c1ccccc1)/C=C/C=C/c1ccccc1 ; OC(/C=C/C=C/c1ccccc1)/C=C/C=C/c1ccccc1 ; OC(CC/C=C/c1ccccc1)CC/C=C/c1ccccc1]

The α,β-unsaturated ketones benzylideneacetone 38 and (1E,3E,6E,8E)-1,9-diphenylnona-1,3,6,8-tetraen-5-one 41 were studied in the TH catalyzed by complexes 13, 14 and 16 in 2-propanol. The commercially available compound 38 can also be prepared by reaction of benzaldehyde and acetone, whereas the ketone 41 was prepared by double aldol type condensation between trans-cinnamaldehyde and acetone. Compounds 38 and 41 are also formed as side products during the TH of benzaldehyde and trans-cinnamaldehyde, respectively in basic 2-propanol, catalyzed by complexes 13-18.

Allylic alcohols 39 and 42 were obtained by NaBH₄ reduction of 38 and 41 and were available as analytical standards.

Catalytic runs were carried out at a molar substrate to complex ratio as indicated in Table 3. Substrate concentration 0.1 M and a base to complex ratio as indicated in the table. 1 mmol of benzylideneacetone 38 or 1 mmol of (1E,3E,6E,8E)-1,9-diphenylnona-1,3,6,8-tetraen-5-one 41 was dissolved in 10 mL of 2-propanol and the solution was purged with argon followed by three vacuum/argon cycles. The required complex quantity and 6.9 mg (0.05 mmol) of K₂CO₃ was charged and the reaction mixture was heated in a preheated oil bath to reflux (82° C.). After the reaction time, the solvent was evaporated under vacuum, the crude mixture dissolved in CDCl₃ and analyzed by ¹H-NMR spectroscopy.

TABLE 3
Catalytic transfer hydrogenation of 38 and 41 (0.1M) with complexes 13, 14, 16
(S/C = 1000-5000) and K₂CO₃ in 2-propanol.

| | | | S/C | S/Base | Base/C | Time | Conv. | | |
|---|---|---|---|---|---|---|---|---|---|
| Entry | Substrate | Complex | molar | molar | molar | (min) | (%)^{a} | (%) | (%) |
| 1 | 38 | 14 | 5000 | 50/1 | 100/1 | 45 | 93 | 0 | 93 |
| | (146 mg) | (0.16 mg) | | | | | | 39 | 40 |
| 2 | 41 | 13 | 1000 | 50/1 | 20/1 | 15 | 100 | 0 | 95 |
| | (286 mg) | (0.8 mg) | | | | | | 42 | 43 |
| 3 | 41 | 14 | 1000 | 50/1 | 20/1 | 15 | 100 | 0 | 95 |
| | (286 mg) | (0.8 mg) | | | | | | 42 | 43 |
| 4 | 41 | 16 | 5000 | 50/1 | 100/1 | 45 | 99 | 0 | 82 |
| | (286 mg) | (0.16 mg) | | | | | | 42 | 43 |
| 5 | 41 | 16 | 5000 | 50/1 | 100/1 | 45 | 100 | 0 | 95 |
| | (286 mg) | (0.16 mg) | | | | | | 42 | 43 |

^{a}The conversion was determined by NMR analysis.

None of the reaction allowed identifying any allylic alcohol 39 or 42 in the reaction product. Only the alcohols 40 and 43 were isolated. Mechanistically either a 1,4-addition pathway (no formation of allylic alcohols at any time) or a very fast catalytic allylic alcohol isomerisation step is observed.

### Example 26

Reduction of Ketones with Hydrogen.

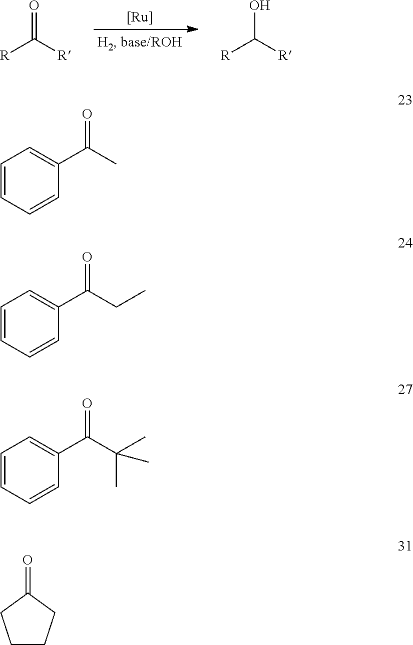

In an autoclave glass insert at 40° C., 5 mmol of substrate and the required amount of base (Tables 4 and 5) were dissolved in the alcohol solvent (total reaction volume 10 mL) and with agitation switched on, purged with nitrogen (pressurise to 3 bar and vent to ambient pressure). The required amount of a complex stock solution in the reaction solvent was added. Directly after the addition, the mixture was purged three times with nitrogen (pressurise to 3 bar and vent to ambient pressure). Then it was purged twice with hydrogen (pressurise to 5 bar and vent to ambient pressure) and then kept pressurised at reaction pressure for the time defined as reaction duration. After this, the autoclave was vented and the product analysed by GC.

TABLE 4
Catalytic hydrogenation of ketones (0.5M) with
complexes 13-15 and 18 (S/C = 10000) under 5 bar of
H₂ and a base (2 mol %) in methanol at 40° C.

| | | | | | | Conv. to |
|---|---|---|---|---|---|---|
| Entry | Complex | Ketone | Base | t (min) | | alcohol (%)^{a} |
| 1 | 13 | 23 | NaOMe | 20 | | 95 |
| 2 | 14 | 23 | NaOMe | 60 | | 96 |
| 3 | 15 | 23 | NaOMe | 20 | | 95 |
| 4 | 15 | 23 | NaOH | 30 | | 96 |
| 5 | 15 | 23 | KOH | 15 | | 95 |
| 6 | 18 | 23 | KOH | 60 | | 94 |
| 7 | 15 | 24 | KOH | 15 | | 93 |
| 8 | 13 | 27 | KOH | 120 | | 8 |
| 9 | 13 | 27 | KOH | 20 | h | 25 |
| 10 | 15 | 31 | KOH | 120 | | 91 |

^{a}As determined by GC analysis the conversion of the substrate and the conversion to alcohol was the same in all cases. There is no decomposition of the substrate under reaction conditions, allowing e.g. the use of higher hydrogen pressure and the use of longer reaction times. Higher temperatures (i.e. 70° C.) can be applied without inducing decomposition of the substrate

TABLE 5
Catalytic hydrogenation of acetophenone (23) (0.5M) with complex 15 at
40° C. with different solvent base combinations.

| | | | | | Conv. to alcohol |
|---|---|---|---|---|---|
| Entry | Solvent | Base | S/C | t (min) | (%)^{a} |
| 1 | EtOH | KOtBu | 5000 | 30 | 2 |
| 2 | EtOH | KOtBu | 5000 | 180 | 2 |
| 3 | MeOH | KOtBu | 25000 | 300 | 34 |
| 4 | MeOH | KOtBu | 25000 | 420 | 38 |
| 5 | MeOH | KOH | 10000 | 15 | 95 |
| 10 | MeOH | KOH | 10000 | 30 | 98 |

^{a}As determined by GC analysis the conversion of the substrate and the conversion to alcohol was the same in all cases. There is no decomposition of the substrate under reaction conditions, allowing e.g. the use of higher hydrogen pressure and the use of longer reaction times. Higher temperatures (i.e. 70° C.) can be applied without inducing decomposition of the substrate

The complexes display high catalytic activity in the hydrogenation of ketones in basic alcohol media. Strong solvent effects (MeOH vs EtOH), choice of base effects are evident from the data. No decomposition of the substrate is observed under reaction condition. Compared to transfer hydrogenation the reactions can be run more volume efficiently i.e. at higher concentration of substrate.

### Example 27

Reduction of Me-Benzoate (44) with Hydrogen.

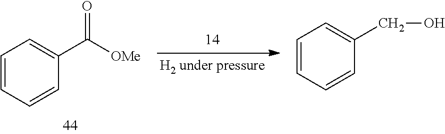 [SMILES: COC(=O)c1ccccc1 ; OCc1ccccc1]

A 10 mL glass tube was charged with complex (0.01 mmol, S/C 500/1), loaded in a Biotage Endevaour, purged with nitrogen five times by pressurizing to 2 bar and releasing pressure. Methyl benzoate (5 mmol, 0.63 mL), 1M KOtBu solution in t-BuOH (0.5 mL) and solvent (4.37 mL) were injected. The vessel was purged again with nitrogen three times, five times under stirring and a further five time with hydrogen (by pressurizing to 28 bar and releasing pressure). The pressure was set at 28 bar of hydrogen and the reaction was stirred (600 rpm) at 50° C. for 16 hours. After cooling to room temperature the pressure was released and the reaction was sampled (2 mL MeOH and 0.5 mL water were added). An aliquot of 100 μL was diluted in 1 mL acetonitrile and analyzed by GC (Table 6).

TABLE 6
Catalytic hydrogenation of Me-benzoate (44)
with complex 14 (0.2 mol %) in the presence of KOtBu at 50° C.
with H₂ (28 bar) in different solvents.

| | | Solvent/ | | Benzyl | Benzyl | |
|---|---|---|---|---|---|---|
| | | 10% | Conversion | Alcohol | benzoate | Others |
| Entry | Complex | tBuOH | (%) | (%) | (%)^{[A]} | (%) |
| 1 | 14 | MeTHF | 75 | 63 | 10 | 2 |
| 2 | 14 | Toluene | 55 | 33 | 14 | 8 |

^{[A]}Benzylbenzoate is the benzyl alcohol ester of benzoic acid and it formation requires the conversion of methyl benzoate by hydrogenation.

The pincer complex 14 catalyses the ester hydrogenation.

### Example 28

Transfer Hydrogenation with 2-Propanol as Hydride Donor on Aromatic Aldehydes

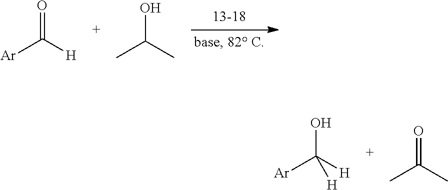

An aldehyde selected from 45-49 (1 mmol), K₂CO₃ (6.9 mg; 0.05 mmol) and 2-propanol were introduced in a Schlenk, subjected to three vacuum-argon cycles and the tube was put in an oil bath at 90° C. From a 250 μM solution of the ruthenium complex in 2-propanol, the required quantity of complex were added to the refluxing mixture to reach a final volume of 10 mL. The reaction was sampled by removing an aliquot of the reaction mixture, adding diethyl ether (1/1 in volume) and after filtration over a silica pad, the conversion was determined by GC analysis. For solid and high boiling compounds, the solvent was evaporated by gently heating under vacuum, the crude mixture was dissolved in CDCl₃ and analyzed by ¹H-NMR spectroscopy;

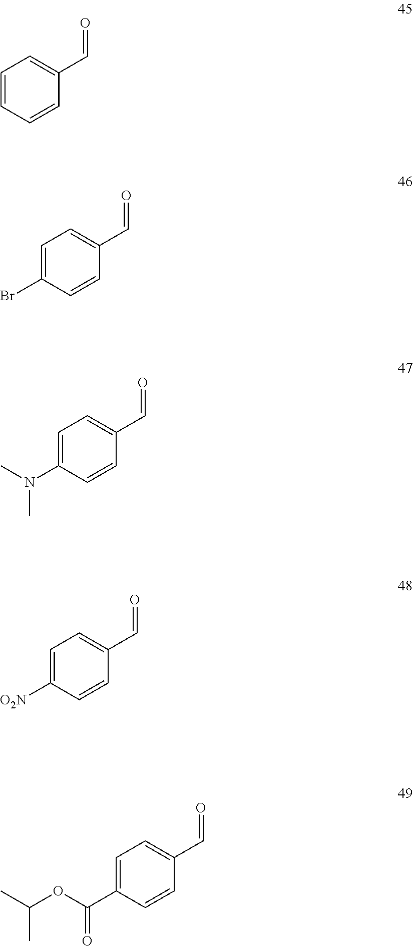

TABLE 7
TH of aromatic aldehydes (0.1M) catalyzed by
complexes 13-18 and K₂CO₃ (5 mol %) in 2-propanol at 82° C.

| | | | Time | Conv. | Alcohol | By-products |
|---|---|---|---|---|---|---|
| Aldehyde | Complex | S/C | (h) | (%)^{[a]} | (%) | (%) |
| 45 | 13 | 2000 | 2 | 100 | 99 | 1 |
| | 14 | 2000 | 1.5 | 100 | >99 | <1 |
| | 15 | 2000 | 1.25 | 99 | 98 | 1 |
| | 16 | 2000 | 5 | 99 | 98 | 1 |
| | 17 | 2000 | 5 | 99 | 98 | 1 |
| | 18 | 2000 | 1.25 | 99 | 98 | 1 |
| 46 | 13 | 2000 | 0.5 | 98 | 78 | 20 |
| | 14 | 2000 | 2 | 100 | 82 | 18 |
| | 15 | 2000 | 0.5 | 98 | 98 | <1 |
| | 16 | 2000 | 3 | 67 | 54 | 23 |
| | 17 | 2000 | 1 | 100 | 81 | 19 |
| | 18 | 2000 | 0.5 | >99 | >99 | <1 |
| 47 | 13 | 5000 | 1.5 | 95 | 95 | <1 |
| | 13 | 10000 | 3 | 98 | 98 | <1 |
| | 14 | 2000 | <0.5 | 98 | 98 | <1 |
| | 14 | 5000 | 0.5 | 98 | 98 | <1 |
| | 14 | 10000 | 1.5 | 97 | 97 | <1 |
| | 14 | 20000 | 3 | 98 | 98 | <1 |
| | 15 | 5000 | 1.5 | 92 | 92 | <1 |
| | 15 | 10000 | 3 | 98 | 98 | <1 |
| | 16 | 2000 | 2 | 98 | 98 | <1 |
| | 17 | 2000 | 2 | 99 | 99 | <1 |
| | 18 | 2000 | 2 | 98 | 98 | <1 |
| 48 | 14 | 2000 | 2 | 41 | 36 | 5 |
| | 14 | 500 | 2 | 80 | 70 | 10 |
| 49 | 13 | 2000 | 5 | 52 | 52 | <1 |
| | 14 | 2000 | 5 | 75 | 75 | <1 |
| | 15 | 2000 | 0.75 | 95 | 95 | <1 |
| | 16 | 2000 | 6 | 33 | 33 | <1 |
| | 17 | 2000 | 5 | 53 | 53 | <1 |
| | 18 | 2000 | 1 | 96 | 96 | <1 |

^{[a]}The conversion was determined by GC analysis or by ¹H-NMR spectroscopy.

With complexes 13-18 the transfer hydrogenation with 2-propanol as hydride donor on aromatic aldehydes benefits from using K₂CO₃ as base. This allows a reaction temperature of 82° C. (reflux of solvent) with byproduct formation <1% for benzaldehyde 45. Typically, in the reduction of benzaldehyde 45 with 4 mol % of i-PrONa as base and at a temperature of 50° C. to limit by-product formation 45 is completely consumed within 2 hours. 8-15% of byproducts are observed under these conditions.

TABLE 8
Transfer hydrogenation of aldehydes. Comparative examples catalyzed by
complexes RuCl₂(dppb)(AMPY) (50)^{[a]} and RuCl₂(dppf)(AMPY) (51)^{[b]}
with K₂CO₃ (5 mol %), aldehyde 0.1M in 2-propanol at 82° C.

| | | | Time | Conv. | Alcohol | By-products |
|---|---|---|---|---|---|---|
| Aldehyde | Complex | S/C | (h) | (%)^{[c]} | (%) | (%) |
| 45 | 50 | 2000 | 1.75 | 98 | 92 | 6 |
| | 51 | 2000 | 4 | 85 | 74 | 11 |
| | 50 | 5000 | 5 | 43 | 39 | 4 |
| | 51 | 5000 | 5 | 59 | 49 | 10 |
| 46 | 50 | 2000 | 0.5 | 98 | 97 | 1 |
| | 51 | 2000 | 2.5 | 92 | 62 | 30 |
| 47 | 50 | 5000 | 1 | 99 | >98 | <1 |
| | 50 | 10000 | 4.5 | 96 | 89 | 6 |
| | 51 | 2000 | 0.5 | 98 | >97 | <1 |
| | 51 | 5000 | 4 | 52 | 49 | 3 |
| 48 | 50 | 2000 | 12 | 0 | 0 | 0 |
| | 51 | 2000 | 12 | 0 | 0 | 0 |

^{[a]}W. Baratta, E. Herdtweck, K. Siega, M. Toniutti, P. Rigo, Organometallics 2005, 24, 1660.
^{[b]}E. Putignano, G. Bossi, P. Rigo, W. Baratta, Organometallics 2012, 31, 1133.
^{[c]}The conversion was determined by GC analysis or by ¹H-NMR spectroscopy.

These complexes are active on commercial grade aldehydes that have not been distilled prior to the reaction. Aldehydes are known to form several side products that can be detrimental to catalytic reactivity. The examples in Table 7 and 8, therefore, demonstrate robust catalytic activity under non-optimal conditions on unpurified substrates.

The reduction of aromatic aldehydes is more selective with pincer complexes 13-18 of the present invention in comparison to the non-pincer complexes DPPB RuCl₂ AMPY 50 and DPPF RuCl₂ AMPY 51 . . . RuCl₂(dppb)(AMPY) (50) and RuCl₂(dppf)(AMPY) (51) are not able to reduce the aldehyde 49, containing a benzoic ester group. This substrate inhibition is not found for the more robust complexes 13-18 using the same batch of 49.

### Example 29

Transfer Hydrogenation with 2-Propanol as Hydride Donor on Trans-Cinnamaldehyde (52) as Example of an α,β-Unsaturated Aldehyde

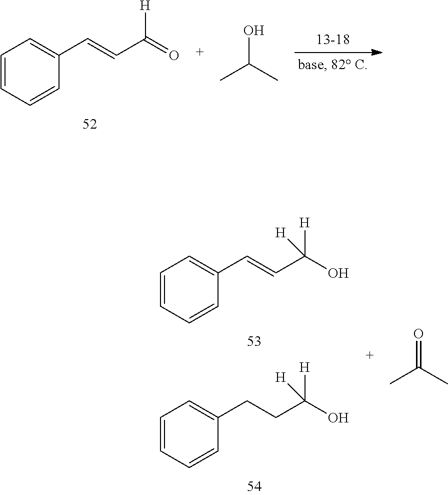

Trans-cinnamaldehyde 52 (1 mmol), K₂CO₃ (6.9 mg; 0.05 mmol) and 2-propanol were introduced in a Schlenk tube, subjected to three vacuum-argon cycles and the tube was put in an oil bath at 90° C. From a 250 μM solution of the ruthenium complex in 2-propanol, the required quantity of complex was added to the refluxing mixture to reach a final volume of 10 mL. At the end of reaction the solvent was evaporated by gently heating under vacuum, the crude mixture was dissolved in CDCl₃ and analyzed by ¹H-NMR spectroscopy.

TABLE 9
TH of trans-cinnamaldehyde (52) (0.1M) catalyzed by complexes 13-18
and 50, 51 with K₂CO₃ (5 mol %) in 2-propanol at 82° C.

| | | Time | Conv | | By-products | |
|---|---|---|---|---|---|---|
| Complex | S/C | (h) | (%)^{[a]} | 53 (%) | (%) | 54 (%) |
| 13 | 5000 | 1 | 99 | 89 | 10 | 10 |
| 13 | 10000 | 6.5 | 68 | 59 | 9 | 1 |
| 14 | 5000 | 1 | 99 | 90 | 9 | 7 |
| 14 | 10000 | 6.5 | 98 | 84 | 14 | 4 |
| 15 | 5000 | 0.5 | 96 | 77 | 19 | 19 |
| 15 | 10000 | 4 | 98 | 80 | 18 | 3 |
| 16 | 5000 | 4 | 93 | 73 | 20 | 3 |
| 16 | 10000 | 4 | 96 | 77 | 19 | 4 |
| 17 | 5000 | 4 | 93 | 73 | 20 | 3 |
| 17 | 10000 | 4 | 96 | 77 | 19 | 4 |
| 18 | 5000 | 1 | 98 | 84 | 14 | 5 |
| 18 | 10000 | 4 | 57 | 44 | 13 | 2 |
| 50 | 2000 | 3 | 78 | 77 | 1 | 1 |
| 51 | 5000 | 3 | 11 | 10 | 1 | 1 |

^{[a]}The conversion was determined by GC analysis or by ¹H-NMR spectroscopy.

Trans-cinnamaldehyde (52) is efficiently reduced by complexes 13-19. The non-pincer complexes 50 and 51 are less efficient. For mostcomplexes, the amount of formation of the saturated alcohol 54 can be reduced by using lower complex loadings. It is likely that the intermediate substrate that forms 54 is the saturated ketone. The saturated ketone can be produced either by the catalyzed isomerization of an allylic alcohol intermediate (known to be efficiently catalyzed by non-pincer complex 51) or following a 1,4 addition pathway by converting the enol intermediate to the saturated ketone.

### Example 30

Transfer Hydrogenation in a Biphasic System with Formate Salts as Hydride Donor on Ketone Substrates

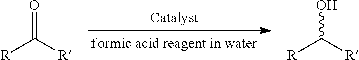

In a Schlenk flask a 0.5M solution of the substrate in toluene (5 mL) was prepared and degassed by 3 vacuum/argon cycles. 5 mL of an argon saturated aqueous stock solution containing the formic acid reagent was added. A paraffin filled bubbler was attached to the Schlenk flask to vent any CO₂ produced. The Schlenk flask was placed in an preheated oil bath at 90° C. and the mixture was vigorously stirred for the required time. ¹H NMR and GC was used to assay the reaction mixtures.

TABLE 10
TH of ketones (0.5M) catalyzed by
complexes 14 and 50, 51 with HCO₂NH₄^{[A]}

| | | | | | | By- |
|---|---|---|---|---|---|---|
| | Com- | | | Time | Alcohol | products |
| Ketone | plex | S/C | Reagent | (h) | (%) | (%) |
| 23 | 50 | 2000 | NH₄-formate^{[B]} | 24 | 1 | 0 |
| | 51 | 2000 | NH₄-formate^{[B]} | 24 | 2 | 0 |
| | 50 | 1000 | NH₄-formate^{[B]} | 24 | 5 | 0 |
| | 51 | 1000 | NH₄-formate^{[B]} | 24 | 4 | 0 |
| 23 | 14 | 2000 | NH₄-formate^{[B]} | 4.5 | 94 | 0 |
| 29 | 14 | 2000 | NH₄-formate^{[B]} | 11 | 50 | 0 |
| 29 | 14 | | Na-formate^{[C]} | | 89 | 0 |
| | 14 | 2000 | (5 NEt₃ + | 24 | 69 | 0 |
| | | | 1 HCOOH)^{[D]} | | | |
| | 14 | 2000 | (5 NEt₃ + | 36 | 90 | 0 |
| | | | 1 HCOOH)^{[E]} | | | |

^{[A]}23 is acetophenone, 29 is benzophenone, 50 is RuCl₂(dppb)(AMPY) and 51 is RuCl₂(AMPY)(dppf).
^{[B]}2 molar equivalents of NH₄-formate.
^{[C]}2 molar equivalents of Na-formate.
^{[E]}2 molar equivalents of formic acid.
^{[F]}5 molar equivalents of formic acid.

The pincer complex 14 reduces the ketone substrates most efficiently and with the lowest amount of reagent when NH₄-formate is used as hydride transfer reagent. The non-pincer complexes RuCl₂(dppb)(AMPY) (50) and RuCl₂(dppf)(AMPY) (51) are poor catalysts with formate reagents.

### Example 31

Transfer Hydrogenation in a Biphasic System with Formate Salts as Hydride Donor on Aldehyde Substrates

The selected aldehyde (2.5 mmol), HCOONH₄ (10 mmol, 0.63 g) and complex (e.g. 1.25 μmol, 1 mg; S/C=2000) are transferred into a 50 ml Schlenk tube. Then toluene (1.2 ml) and water (5 ml) are sequentially added. The biphasic mixture is subjected to four vacuum-argon cycles under vigorous stirring and then put into an oil bath at 90° C. for the desired time. The reaction is sampled by removing 1 ml of the mixture, diethyl ether (4 ml) is added, the organic phase separated, dried over MgSO₄, filtered and the solvent gently removed under reduced pressure. The crude residue was dissolved with CDCl₃ and analyzed by ¹H-NMR. Alternatively, the dried organic fraction is filtered over a short silica pad and the conversion determined by GC analysis.

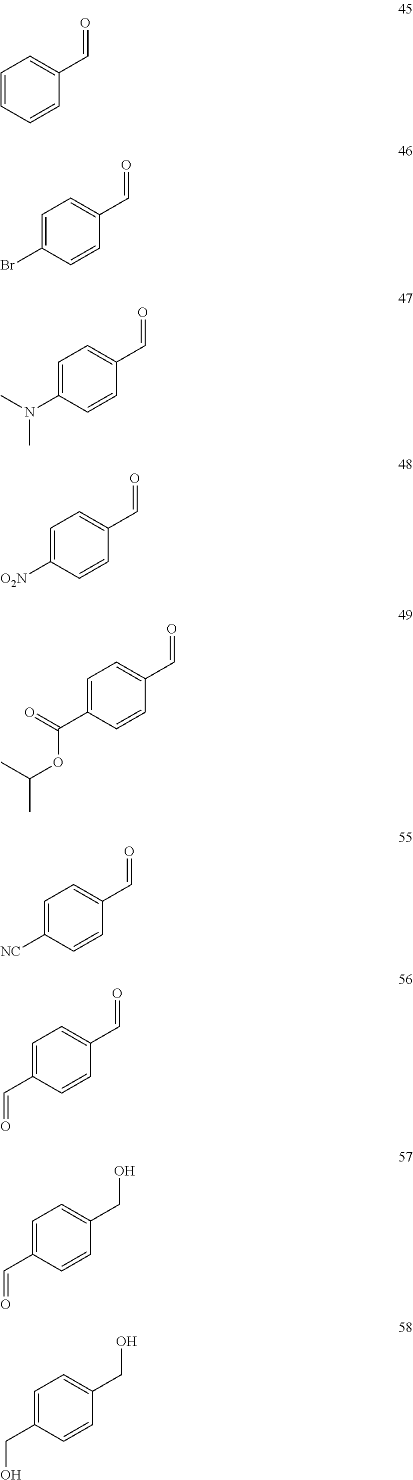

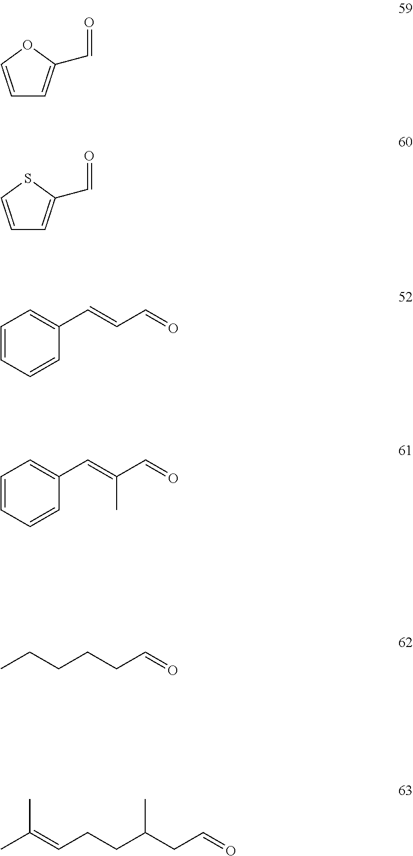

TABLE 11
TH of aldehydes catalyzed by complexes 13-15 with HCO₂NH₄ in toluene/H₂O at 90° C.

| | | | | | | | By- |
|---|---|---|---|---|---|---|---|
| | | | Substrate | NH₄-formate | Time | Alcohol | products |
| Aldehyde | Complex | S/C | molar | molar; equivalents | (h) | (%) | (%) |
| 45 | 13 | 5000 | 0.5 | 1; 2 | 16 | 60 | 0 |
| | 13 | 5000 | 0.5 | 1; 2 | 22 | 76 | 0 |
| | 14 | 5000 | 0.5 | 1; 2 | 15 | 96 | 0 |
| | 14 | 5000 | 0.5 | 1; 2 | 24 | 97 | 0 |
| | 14 | 5000 | 1.0 | 1; 2 | 15 | 86 | 0 |
| | 14 | 5000 | 1.0 | 1; 2 | 24 | 95 | 0 |
| | 14 | 5000 | 0.5 | 1; 4 | 15 | 96 | 0 |
| | 14 | 5000 | 0.5 | 1; 4 | 24 | 96 | 0 |
| | 14 | 5000 | 1.0 | 1; 4 | 15 | 96 | 0 |
| | 14 | 5000 | 1.0 | 1; 4 | 24 | 96 | 0 |
| | 14 | 20000 | 2.0 | 2; 4 | 24 | 94 | 0 |
| | 14 | 20000 | 2.0 | 2; 4 | 48 | 96 | 0 |
| | 15 | 5000 | 0.5 | 1; 2 | 16 | 96 | 0 |
| | 15 | 10000 | 2.0 | 1; 2 | 20 | 86 | 0 |
| | 15 | 20000 | 2.0 | 1; 2 | 40 | 96 | 0 |
| 46 | 14 | 2000 | 0.5 | 1; 2 | 10 | 97 | 0 |
| | 14 | 20000 | 2.0 | 2; 4 | 24 | 62 | 0 |
| | 14 | 20000 | 2.0 | 2; 4 | 48 | 72 | 0 |
| 48 | 14 | 2000 | 0.5 | 2; 4 | 10 | >99 | 0 |
| 55 | 14 | 2000 | 0.5 | 1; 2 | 3.5 | >99 | 0 |
| 56 | 14 | 2000 | 0.5 | 1; 1.5 | 9 | 57: 83 | 0 |
| | | | | | | 58: 17 | |
| | 14 | 2000 | 0.5 | 1; 2 | 10 | 57: 71 | 0 |
| | | | | | | 58: 21 | |
| | 14 | 2000 | 0.5 | 1; 4 | 10 | 57: 0 | 0 |
| | | | | | | 58: 99 | |
| 52 | 14 | 2000 | 0.5 | 1; 2 | 10 | 97 | 54: 10 |
| | 14 | 5000 | 2.0 | 2; 4 | 16 | 78 | 0 |
| | 14 | 5000 | 2.0 | 2; 4 | 24 | 86 | 0 |
| | 14 | 5000 | 2.0 | 2; 4 | 48 | 97 | 0 |
| | 14 | 5000 | 2.0 | 4; 4 | 16 | 84 | 0 |
| | 14 | 5000 | 2.0 | 4; 4 | 24 | 91 | 0 |
| | 14 | 5000 | 2.0 | 4; 4 | 48 | 94 | 0 |
| | 14 | 10000 | 0.5 | 1; 2 | 24 | 38 | 0 |
| | 14 | 10000 | 0.5 | 1; 2 | 38 | 49 | 0 |

With complexes 13-15 the transfer hydrogenation of aldehydes with NH₄-formate is an improvement compared to using 2-propanol as hydride donor and K₂CO₃ as base (examples 28 and 29). The use of less complex (higher S/C ratio) is possible and less by-products are formed. It is important to note that no primary amines are produced by reductive amination of the aldehyde. Interestingly, the presence of the toluene solvent as co-solvent is not entirely required, as shown in the table below. Toluene was not added to the reactions carried out on a 2.5 mmol substrate scale.

TABLE 12
TH of aldehydes catalyzed by complex 14 with HCO₂NH₄ in H₂O at 90° C.

| | | | | | | | By- |
|---|---|---|---|---|---|---|---|
| | | | Substrate | NH₄-formate | Time | Alcohol | products |
| Aldehyde | Complex | S/C | molar | molar; equivalents | (h) | (%) | (%) |
| 45 | 13 | 5000 | 0.5 | 1; 2 | 16 | 60 | 0 |
| | 13 | 5000 | 0.5 | 1; 2 | 22 | 76 | 0 |
| | 14 | 2000 | 2.5 mmol | 1; 2 | 2 | 50 | 0 |
| | 14 | 2000 | 2.5 mmol | 1; 2 | 4 | 76 | 0 |
| | 14 | 2000 | 2.5 mmol | 1; 2 | 7 | 97 | 0 |
| | 14 | 5000 | 2.5 mmol | 1; 2 | 14 | 53 | 0 |
| | 14 | 5000 | 2.5 mmol | 1; 4 | 24 | 97 | 0 |
| | 14 | 5000 | 2.5 mmol | 2; 4 | 24 | 90 | 0 |
| | 14 | 5000 | 0.5 | 1; 2 | 15 | 96 | 0 |
| | 14 | 5000 | 0.5 | 1; 2 | 24 | 97 | 0 |
| | 14 | 5000 | 1.0 | 1; 2 | 15 | 86 | 0 |
| | 14 | 5000 | 1.0 | 1; 2 | 24 | 95 | 0 |
| | 14 | 5000 | 0.5 | 1; 4 | 15 | 96 | 0 |
| | 14 | 5000 | 0.5 | 1; 4 | 24 | 96 | 0 |
| | 14 | 5000 | 1.0 | 1; 4 | 15 | 96 | 0 |
| | 14 | 5000 | 1.0 | 1; 4 | 24 | 96 | 0 |
| | 14 | 20000 | 2.0 | 2; 4 | 24 | 94 | 0 |
| | 14 | 20000 | 2.0 | 2; 4 | 48 | 96 | 0 |
| | 15 | 5000 | 0.5 | 1; 2 | 16 | 96 | 0 |
| | 15 | 10000 | 2.0 | 1; 2 | 20 | 86 | 0 |
| | 15 | 20000 | 2.0 | 1; 2 | 40 | 96 | 0 |
| 46 | 14 | 2000 | 2.5 mmol | 1; 2 | 14 | 33 | 6 |
| | 14 | 2000 | 2.5 mmol | 1; 2 | 16 | 61 | 0 |
| | 14 | 2000 | 0.5 | 1; 2 | 10 | 97 | 0 |
| | 14 | 2000 | 2.0 | 2; 4 | 11 | 97 | 0 |
| | 14 | 10000 | 2.0 | 2; 4 | 24 | 24 | 0 |
| | 14 | 20000 | 2.0 | 2; 4 | 24 | 62 | 0 |
| | 14 | 20000 | 2.0 | 2; 4 | 48 | 72 | 0 |
| 47 | 14 | 5000 | 2.0 | 2; 4 | 15 | 96 | 0 |
| 48 | 14 | 2000 | 0.5 | 2; 4 | 10 | >99, 65^{[b]} | 0 |
| 55 | 14 | 2000 | 0.5 | 1; 2 | 3.5 | >99 | 0 |
| | 14 | 2000 | 2.0 | 2; 4 | 3.5 | 99, 65^{[b]} | 0 |
| 56 | 14 | 2000 | 0.5 | 1; 1.5 | 9 | 57: 83 | 0 |
| | | | | | | 58: 17 | |
| | 14 | 2000 | 0.5 | 1; 2 | 10 | 57: 71 | 0 |
| | | | | | | 58: 21 | |
| | 14 | 2000 | 0.5 | 1; 4 | 10 | 57: 0 | 0 |
| | | | | | | 58: 99 | |
| 52 | 14 | 2000 | 0.5 | 1; 2 | 10 | 97 | 54: 10 |
| | 14 | 5000 | 2.0 | 2; 4 | 16 | 78 | 54: 6 |
| | 14 | 5000 | 2.0 | 2; 4 | 24 | 86 | 0 |
| | 14 | 5000 | 2.0 | 2; 4 | 48 | 97 | 54: 12 |
| | 14 | 5000 | 2.0 | 4; 4 | 16 | 84 | 0 |
| | 14 | 5000 | 2.0 | 4; 4 | 24 | 92 | 54: 5 |
| | 14 | 5000 | 2.0 | 4; 4 | 48 | 94 | 54: 7 |
| | 14 | 10000 | 0.5 | 1; 2 | 24 | 38 | |
| | 14 | 10000 | 0.5 | 1; 2 | 38 | 49 | |
| 59 | 14 | 10000 | 2.0 | 4; 4 | 20 | 98 | 0 |
| 60 | 14 | 10000 | 2.0 | 2; 4 | 24 | 65 | 0 |
| 60 | 14 | 10000 | 2.0 | 4; 4 | 24 | 97 | 0 |
| 61 | 14 | 5000 | 2.0 | 4; 4 | 20 | 98, 88^{[b]} | |
| 62 | 14 | 5000 | 2.0 | 2; 4 | 8 | 95 | |
| 63 | 14 | 2000 | 2.0 | 4; 4 | 9 | 96, 79^{[b]} | |

^{[a]}Conversion and product content were determined by GC analysis or by ¹H-NMR spectroscopy.
^{[b]}Isolated yield.

On 2.5 mmol scale, reduction of benzaldehyde 45 (0.5 molar in toluene) at S/C=2000, 90° C. and 4 equivalents of 2M aqueous Na-formate gave only traces of benzylalcohol after 14 hours. Use of 4 equivalents of (NEt₃H)-formate improves the yield to 50% in 22 hours. Use of 5 equivalents of (NEt₃H)-formate on trans-cinnamaldehyde 52 gives after 18 hours 80% of allylic alcohol 53 and 15% of saturated alcohol 54. NH₄-formate is preferred over the other formate reagents.

### Example 32

Reduction of Aldehydes with Hydrogen.

In an autoclave glass insert 10 mmol of substrate and the required KOtBu (2 mol %) were dissolved in the alcohol solvent (4 mL) and with stir agitation switched on, purged with nitrogen (pressurise to 3 bar and vent to ambient pressure). The required amount of a complex stock solution in the reaction solvent was added. Directly after the addition, the mixture was purged three times with nitrogen (pressurise to 3 bar and vent to ambient pressure). Then it was purged twice with hydrogen (pressurise to 13 bar and vent to ambient pressure) and then kept pressurised at reaction pressure for the time defined as reaction duration. After this, the autoclave was vented and the product analysed by GC and ¹HNMR.

TABLE 13
Hydrogenation of 2 molar in methanol solutions of benzaldehyde 45
and trans-cinnamaldehyde 52 catalyzed by complexes
RuCl₂(dppb)(AMPY) (50) and RuCl₂(dppf)(AMPY) (51) and
pincer complexes 13-15. Base KOtBu (2 mol %).
Hydrogenation in a Biotage Endeavour apparatus at 50° C.

| | | | | | | | By- |
|---|---|---|---|---|---|---|---|
| Sub- | Com- | Loading | P (H₂) | Time | Conv. | Alcohol | products. |
| strate^{[a]} | plex | [S/C] | [atm] | [h] | [%]^{[a]} | [%]^{[a]} | [%]^{[a]} |
| 45 | 50 | 1000 | 10 | 3 | 35 | 33 | 2 |
| 45 | 50 | 2000 | 10 | 8 | 22 | 7 | 15 |
| 45 | 51 | 2000 | 10 | 16 | 100 | 98 | 2 |
| 45 | 13 | 10000 | 10 | 8 | 100 | 96 | 4 |
| 45 | 13 | 20000 | 10 | 8 | 100 | 99 | 1 |
| 45 | 14 | 10000 | 10 | 16 | 98 | 98 | 2 |
| 45 | 14 | 40000 | 13 | 32 | 27 | 26 | 1 |
| 45 | 15 | 10000 | 13 | 16 | 63 | 60 | 3 |
| 52 | 50 | 1000 | 10 | 3 | 95 | 87 | 54: 8 |
| 52 | 51 | 2000 | 10 | 8 | 98 | 89 | 54: 9 |
| 52 | 13 | 10000 | 10 | 8 | 99 | 89 | 54: 10 |
| 52 | 13 | 20000 | 10 | 8 | 96 | 75 | 54: 21 |
| 52 | 14 | 10000 | 10 | 8 | 99 | 90 | 54: 11 |

^{[a]}Conversion and product content were determined by GC analysis or by ¹H-NMR spectroscopy.

TABLE 14
Hydrogenation of 2 molar solutions of benzaldehyde 45 catalyzed pincer
complexes 13-15. Base KOtBu (2 mol %). Hydrogenation at S/C = 10000
in a Biotage Endeavour apparatus at 50° C. and 13 bar H₂ for 16 hours.

| | | | Alcohol | By-products. |
|---|---|---|---|---|
| Complex | Solvent | Conv. [%]^{[a]} | [%]^{[a]} | [%]^{[a]} |
| 13 | MeOH | 100 | 96 | 4 |
| 13 | MeOH/EtOH = 3/1 | 100 | 93 | 7 |
| 13 | MeOH/EtOH = 1/1 | 100 | 88 | 12 |
| 13 | MeOH/EtOH = 1/3 | 100 | 86 | 11 |
| 14 | MeOH | 100 | 98 | 2 |
| 14 | MeOH/EtOH = 3/1 | 100 | 97 | 3 |
| 14 | MeOH/EtOH = 1/1 | 100 | 97 | 3 |
| 14 | MeOH/EtOH = 1/3 | 90 | 80 | 10 |
| 14 | EtOH | 100 | 82 | 18 |
| 15 | MeOH | 63 | 60 | 3 |
| 15 | MeOH/EtOH = 3/1 | 23 | 19 | 4 |
| 15 | MeOH/EtOH = 1/1 | 23 | 18 | 5 |
| 15 | MeOH/EtOH = 1/3 | 19 | 16 | 3 |

^{[a]}Conversion and product content were determined by GC analysis or by ¹H-NMR spectroscopy.

TABLE 15
Hydrogenation of methanol solutions of aldehydes catalyzed by
complex 14. Base KOtBu (2 mol %). Hydrogenation in
Parr autoclave at 50° C. and 5 bar H₂.

| | | Loading | | Conv. | Alcohol | By-products. |
|---|---|---|---|---|---|---|
| Substrate^{[a]} | [S] | [S/C] | Time [h] | [%]^{[a]} | [%]^{[a]} | [%]^{[a]} |
| 47 | 1M | 10000 | 1 | 100 | >99 | 0 |
| 47 | 1M | 20000 | 7 | 98 | >97 | 0 |
| 47 | 1M | 40000 | 22 | 98 | >97 | 0 |
| 60 | 1M | 10000 | 1 | 100 | 99 | 1 |
| 60 | 2M | 5000 | 0.66 | 100 | 95 | 5 |
| 61 | 2M | 15000 | 24 | 100 | >99 | <1 |
| 62 | 1M | 5000 | 1.5 | 99 | >90 | <9 |

^{[a]}Conversion and product content were determined by GC analysis or by ¹H-NMR spectroscopy.

The advantage of using the pincer complexes 13-15 compared to using RuCl₂(dppb)(AMPY) (50) and RuCl₂(dppf)(AMPY) (51) is again shown by the hydrogenation data. In the hydrogenation of trans cinnamaldehyde 52 the formation of fully saturated product cannot be suppressed to a similar degree as with the NH₄-formate hydrogenation. Methanol as reaction solvent is clearly preferable over ethanol and methanol/ethanol mixtures.

1. A benzo[h]quinoline compound of formula (1a) or (1b), or salt thereof: 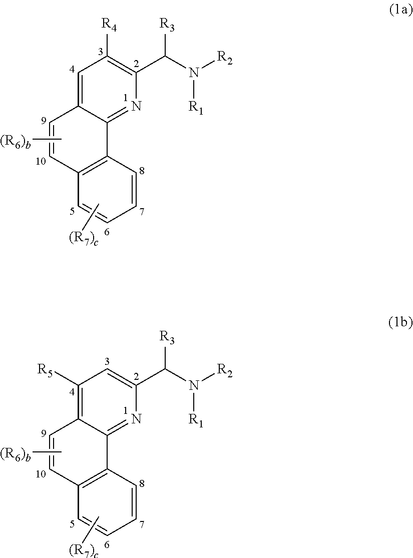
wherein:
R₁ and R₂ are, independently, —H, —OH, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl or substituted C₄₋₂₀-heteroaryl;
R₃ is —H, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, or substituted C₄₋₂₀-heteroaryl;
R₄ is unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, or substituted C₅₋₂₀-aryl;
R₅ is unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, or substituted C₅₋₂₀-aryl;
R₆ is unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″, or —CONR′R″, wherein R′ and R″ are, independently, H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, or substituted C₇₋₂₀-arylalkyl, or R′ and R″ together with the atom to which they are attached form a substituted or unsubstituted C₂₋₂₀-heterocycloalkyl group;
R₇ is unsubstituted —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″, or —CONR′R″, wherein R′ and R″ are, independently, H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, or substituted C₇₋₂₀-arylalkyl, or R′ and R″ together with the atom to which they are attached form a substituted or unsubstituted C₂₋₂₀-heterocycloalkyl group;
b is 0, 1 or 2; and
c is 0, 1, 2, 3 or 4.

2. The compound according to claim 1, wherein the compound of formula (1a) is:

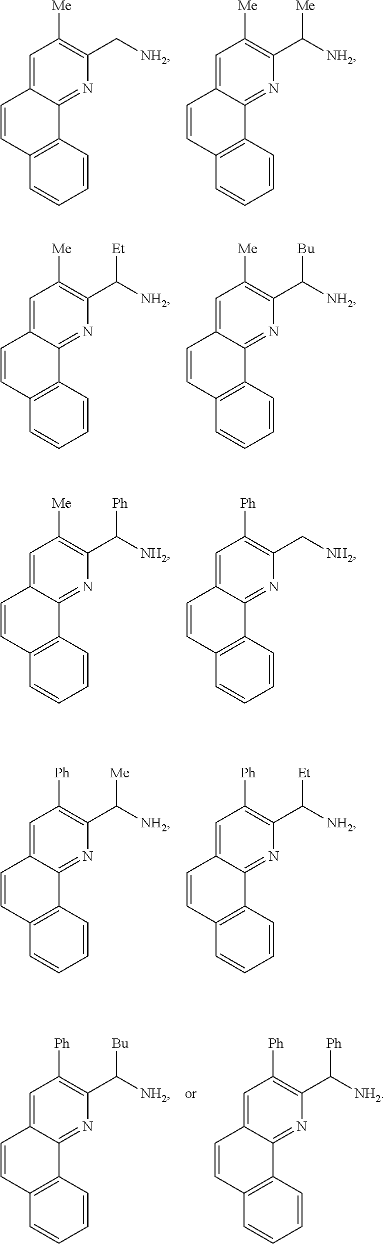

3. The compound according to claim 1, wherein the compound of formula (1b) is:

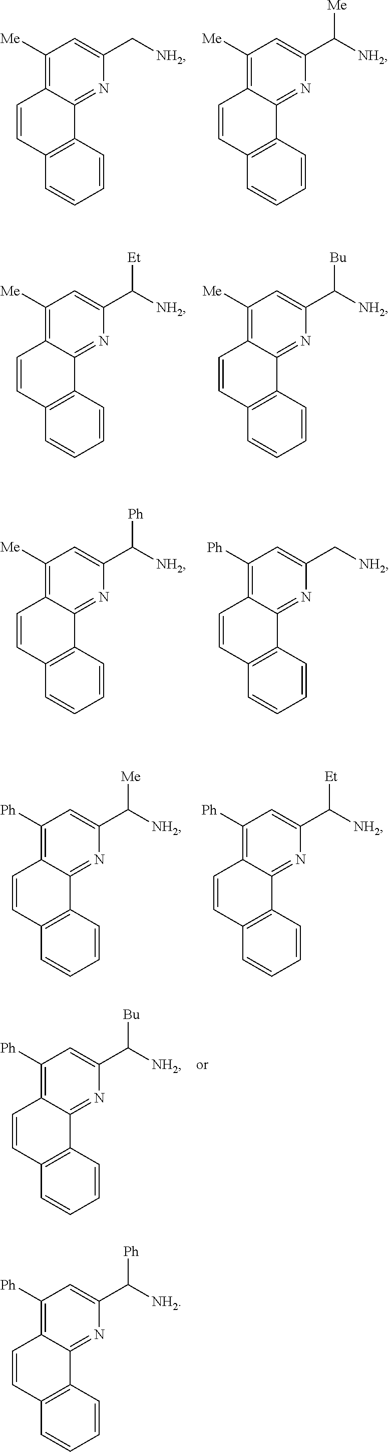

4. A process for preparing a compound of formula (1a) or (1b), the process comprising the step of reacting a compound (4a) or (4b) with a base and a compound of formula (5): 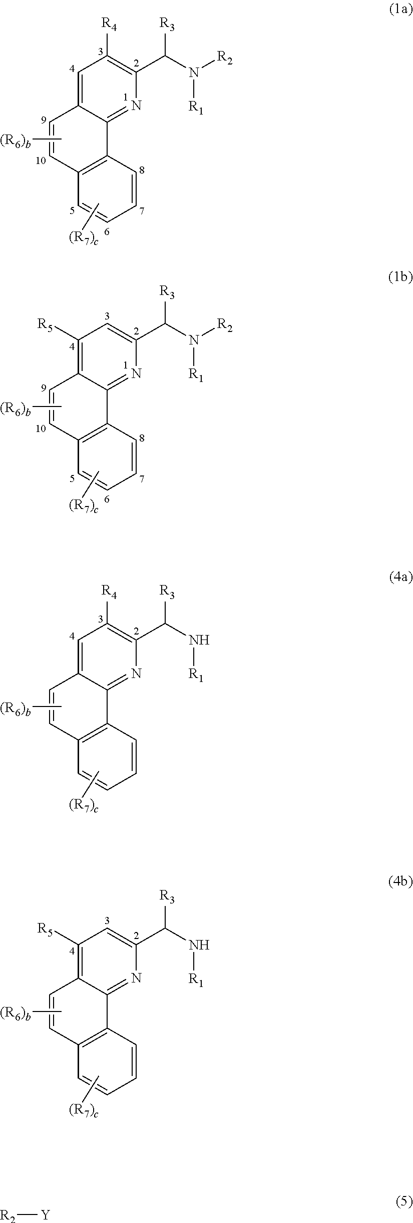
wherein:
R₁ and R₂ are, independently, of —H, —OH, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, or substituted C₄₋₂₀-heteroaryl;
R₃ is —H, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, or substituted C₄₋₂₀-heteroaryl;
R₄ is unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, or substituted C₅₋₂₀-aryl;
R₅ is unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, or substituted C₅₋₂₀-aryl;
R₆ is unsubstituted —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″, or —CONR′R″, wherein R′ and R″ are, independently, H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, or substituted C₇₋₂₀-arylalkyl, or R′ and R″ together with the atom to which they are attached form a substituted or unsubstituted C₂₋₂₀-heterocycloalkyl;
R₇ is —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″, or —CONR′R″, wherein R′ and R″ are, independently, H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, or substituted C₇₋₂₀-arylalkyl, or R′ and R″ together with the atom to which they are attached form a substituted or unsubstituted C₂₋₂₀-heterocycloalkyl;
b is 0, 1 or 2;
c is 0, 1, 2, 3 or 4; and
Y is a leaving group.

5. A process according to claim 4, wherein the compound of formula (4a) or (4b) is prepared by reducing a compound (6a) or (6b) or a salt thereof

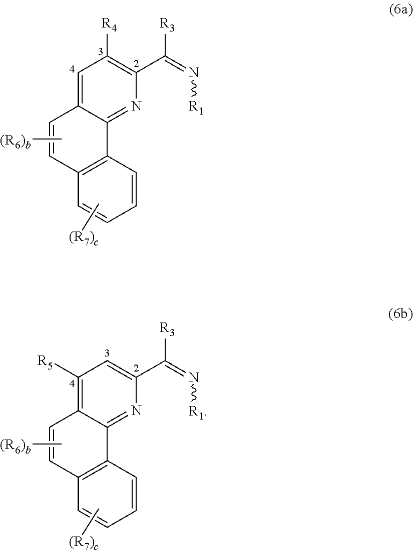

6. A process according to claim 5, wherein the compound (6a) or (6b) or salt thereof is prepared by reacting a compound of formula (7a) or (7b) with a compound of formula (8), or salt thereof, in an alcohol solvent to form compound (6a) or (6b): 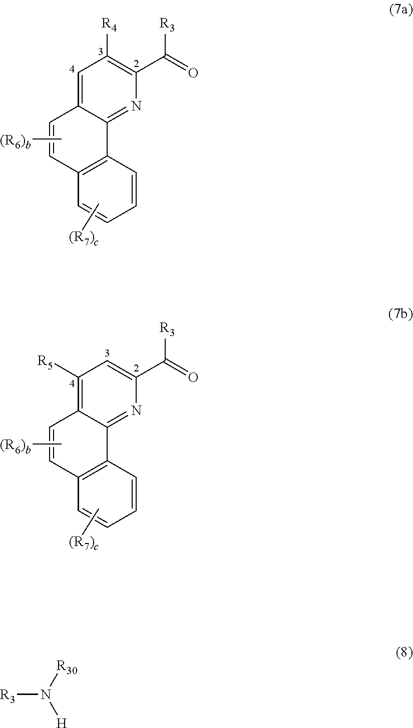
wherein, R₃₀ is —H or —OH.

7. A process according to claim 6, wherein the compound of formula (7a) or (7b) is prepared by:
(a) reacting a compound of formula (9a) or (9b) with a lithiating agent in an ethereal solvent to form lithiated compound (10a) or (10b); and 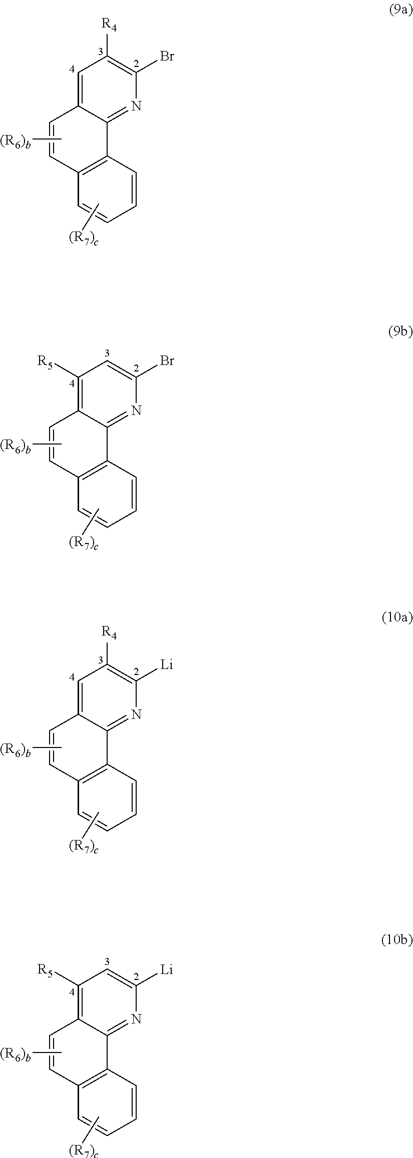
(b) reacting the lithiated compound (10a) or (10b) with a compound of formula (11) to form a compound of formula (7a) or (7b) 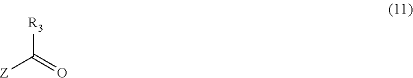
wherein:
Z is —N(alkyl)₂ or -Hal.

8. A process according to claim 7, wherein the compound of formula (9a) or (9b) is prepared by reacting a compound of formula (12a) or (12b) with a halogenating agent in a solvent

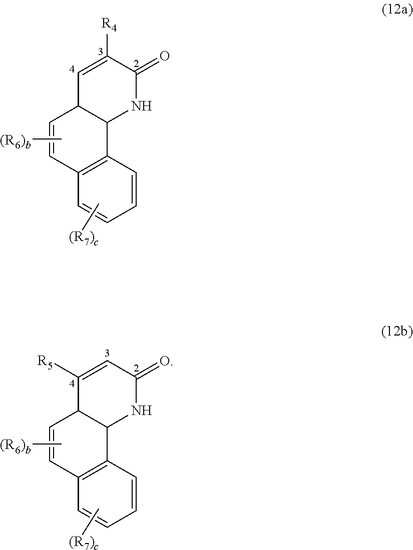

9. A process according to claim 8, wherein the compound of formula (12a) or (12b) is prepared by reacting a compound of formula (13a) or (13b) with an acid

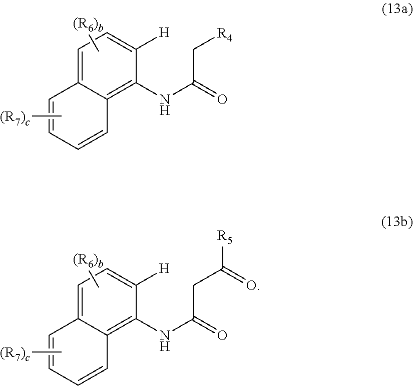

10. A process according to claim 9, wherein the compound of formula (13a) is prepared reacting a naphthylamine of formula (14), or salt thereof, with a compound of formula (15): 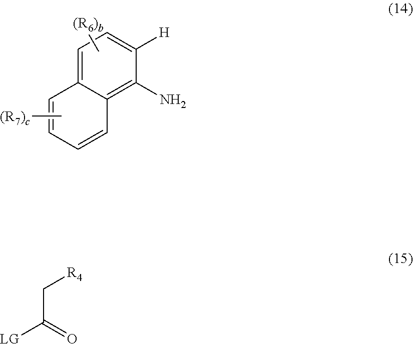
wherein:
LG is a leaving group.

11. A process according to claim 9, wherein the compound of formula (13b) is prepared by reacting a compound of formula (14) with a compound of formula (16) or a compound of formula (17) 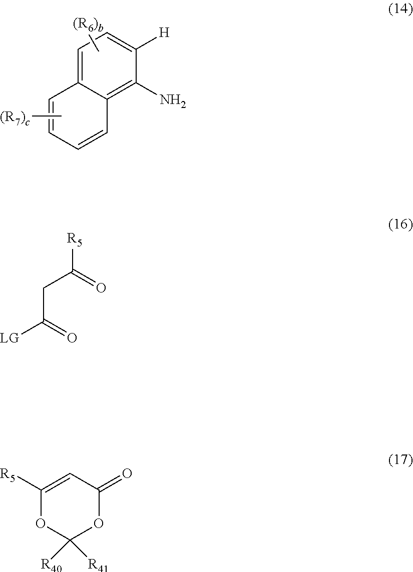
wherein:
R₄₀ and R₄₁ are, independently, unsubstituted alkyl or substituted alkyl, or R₄₀ and R₄₁ are interconnected to form a ring with the carbon to which they are attached; and
LG is a leaving group.

12. A process according to claim 4, wherein the compounds of formulae (1a) and (1b), or salts thereof, are prepared by reducing a compound of formula (20a) or (20b), or salt thereof 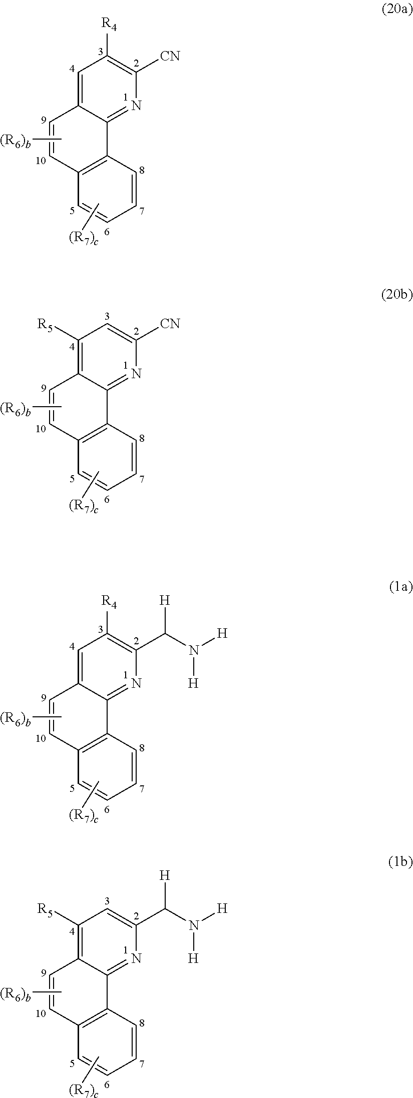
wherein:
R₁, R₂ and R₃ are —H.

13. A process according to claim 12, wherein the compounds of formulae (20a) and (20b) are prepared by cyanating the compound of formulae (9a) or (9b)

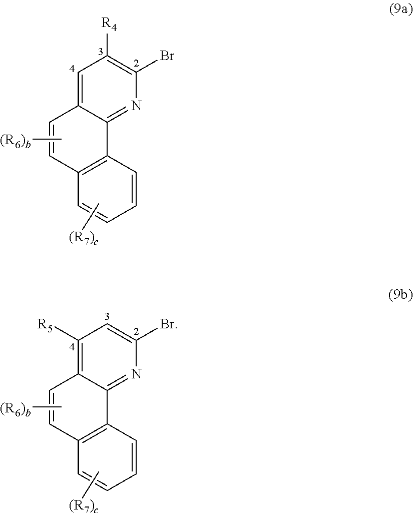

14. A compound which is of formulae (4a), (4b), (6a), (6b), (7a), (7b), (9a), (9b), (12a), (12b), (13a), (13b), (20a) or (20b) 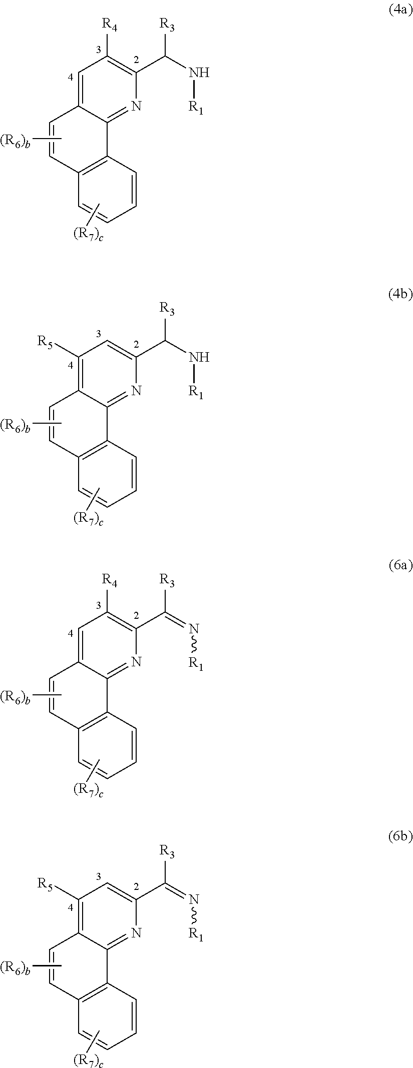 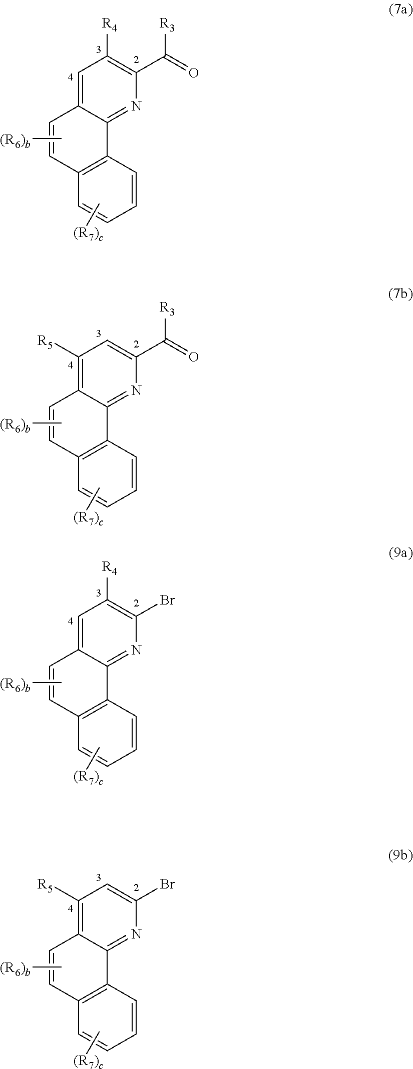 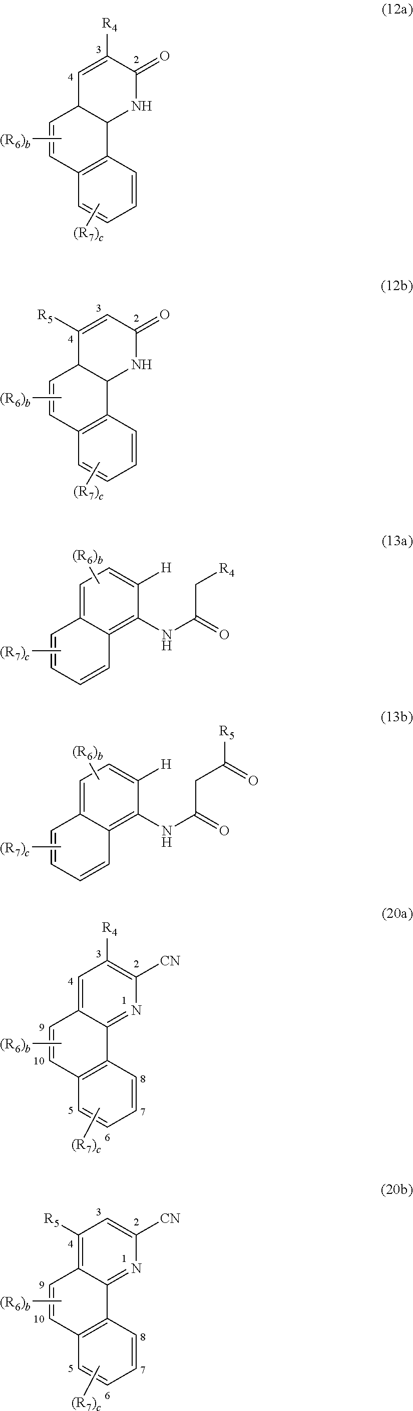
wherein:
R₁ is —H, —OH, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, or substituted C₄₋₂₀-heteroaryl;
R₃ is —H, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, or substituted C₄₋₂₀-heteroaryl;
R₄ is unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, or substituted C₅₋₂₀-aryl;
R₅ is unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, or substituted C₅₋₂₀-aryl;
R₆ is —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″, or —CONR′R″, wherein R′ and R″ are, independently, H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, or substituted C₇₋₂₀-arylalkyl, or R′ and R″ together with the atom to which they are attached form a substituted or unsubstituted C₂₋₂₀-heterocycloalkyl;
R₇ is —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″, or —CONR′R″, wherein R′ and R″ are, independently, H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, or substituted C₇₋₂₀-arylalkyl, or R′ and R″ together with the atom to which they are attached form a substituted or unsubstituted C₂₋₂₀-heterocycloalkyl;
b is 0, 1 or 2;
c is 0, 1, 2, 3 or 4.

15. A transition metal complex of formula (3):
[MX(L¹)ₘ(L²)] (3)
wherein:
M is ruthenium, osmium or iron;
X is an anionic ligand;
L¹ is a monodentate phosphorus ligand or a bidentate bis-phosphorus ligand;
m is 1 or 2, wherein:
when m is 1, L¹ is a bidentate bis-phosphorus ligand;
when m is 2, each L¹ is a monodentate phosphorus ligand; and
L² is a tridentate ligand of formula (2a) or (2b): 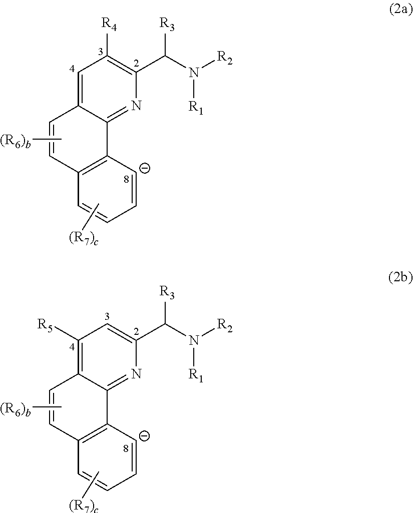
wherein:
R₁ and R₂ are, independently, —H, —OH, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, or substituted C₄₋₂₀-heteroaryl;
R₃ is —H, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl or substituted C₄₋₂₀-heteroaryl;
R₄ is unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, or substituted C₅₋₂₀-aryl;
R₅ is unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, or substituted C₅₋₂₀-aryl;
R₆ is unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″, or —CONR′R″, wherein R′ and R″ are, independently, H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, or substituted C₇₋₂₀-arylalkyl, or R′ and R″ together with the atom to which they are attached form a substituted or unsubstituted C₂₋₂₀-heterocycloalkyl;
R₇ is —H, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″, or —CONR′R″, wherein R′ and R″ are, independently, H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, or substituted C₇₋₂₀-arylalkyl, or R′ and R″ together with the atom to which they are attached form a substituted or unsubstituted C₂₋₂₀-heterocycloalkyl group;
b is 0, 1 or 2; and
c is 0, 1, 2 or 3.

16. A transition metal complex according to claim 15, wherein M is ruthenium.

17. A transition metal complex according to claim 15, wherein L¹ is PR₁₁R₁₂R₁₃, wherein Ru i, R₁₂ and R₁₃ are, independently, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl or substituted C₄₋₂₀-heteroaryl.

18. A transition metal complex according to claim 15, wherein L¹ is a chiral or achiral, monodentate or bidentate phosphorus ligand, wherein the phosphorus atom in the phosphorus ligand is covalently bonded to 3 carbon atoms or n heteroatoms and 3-n carbon atoms, where n=1, 2 or 3.

19. A transition metal complex according to claim 18, wherein the heteroatom is N or O.

20. A transition metal complex according to claim 18, wherein L¹ is an unsubstituted or substituted Binap ligand, PPhos ligand, PhanePhos ligand, QPhos ligand, Josiphos ligand, Bophoz ligand, a Skewphos ligand.

21. A transition metal complex according to claim 18, wherein L¹ is PPh₃, dppf (1,1′-bis(diphenylphosphino)ferrocene), dppp (1,3-bis(diphenylphosphino)propane), dppb (1,4-bis(diphenylphosphino)butane), Dipfc (1,1′-bis(di-isopropylphosphino)ferrocene), or dCyPfc.

22. A process for preparing a transition metal complex of formula (3) of claim 15, the process comprising the step of reacting a transition metal complex, L¹, a compound of formula (1a) or (1b) or salt thereof, and a base in an alcohol solvent,
wherein:
the transition metal complex is [ruthenium (arene) (halogen)₂]₂, [ruthenium (halogen) (P(unsubstituted or substituted aryl)₃)], [osmium (arene) (halogen)₂], [osmium (halogen)₂ (P(unsubstituted or substituted aryl)₃)], or [osmium (N(unsubstituted or substituted alkyl)₃)₄ (halogen)₂]; 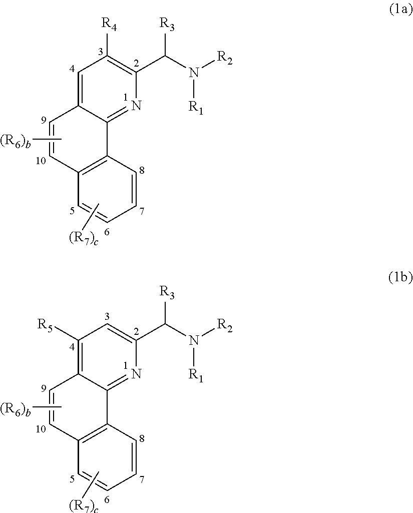
wherein:
R₁ and R₂ are, independently, —H, —OH, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl or substituted C₄₋₂₀-heteroaryl;
R₃ is —H, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl or substituted C₄₋₂₀-heteroaryl;
R₄ unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, or substituted C₅₋₂₀-aryl;
R₅ is unsubstituted C₁₋₂₀-alkyl, substituted C₁₋₂₀-alkyl, unsubstituted C₁₋₂₀-alkoxy, substituted C₁₋₂₀-alkoxy, unsubstituted C₅₋₂₀-aryl, or substituted C₅₋₂₀-aryl;
R₆ is —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″, or —CONR′R″, wherein R′ and R″ are, independently, H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, or substituted C₇₋₂₀-arylalkyl, or R′ and R″ together with the atom to which they are attached form a substituted or unsubstituted C₂₋₂₀-heterocycloalkyl;
R₇ is —CF₃, unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₅₋₂₀-aryl, substituted C₅₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl, substituted C₄₋₂₀-heteroaryl, —NR′R″—COOR′, —S(O)₂OH, —S(O)₂—R′, —S(O)₂NR′R″, or —CONR′R″, wherein R′ and R″ are, independently, H, unsubstituted C₁₋₂₀-alkyl, substituted C₁₋₂₀-alkyl, unsubstituted C₅₋₂₀-aryl, substituted C₅₋₂₀-aryl, unsubstituted C₇₋₂₀-arylalkyl, or substituted C₇₋₂₀-arylalkyl, or R′ and R″ together with the atom to which they are attached form a substituted or unsubstituted C₂₋₂₀-heterocycloalkyl;
b is 0, 1 or 2; and
c is 0, 1, 2, or 3; and
C-8 of the compound of formula (1a) or (1b) is —H.

23. A method of catalysing a reaction, the method comprising the step of reacting a substrate comprising a carbon-oxygen double bond in the presence of a complex of formula (3) of claim 15.

24. The method of claim 23, wherein the reacting is a reduction.

25. The method of claim 24, where the reduction comprises reacting the substrate with hydrogen, deuterium or tritium.

26. The method of claim 24, where the reduction is a transfer hydrogenation.

27. The method of claim 26, wherein the transfer hydrogenation comprises reducing an aldehyde to form a primary alcohol in the presence of a hydrogen donor that is ammonium formate.

28. A method of catalysing a reaction, the method comprising the step of performing the reaction in the presence of a complex of formula (3) of claim 15, wherein the reaction is an isomerization of an allylic alcohol, dehydrogenation reaction, reduction of an alkenyl bond in an α,β-unsaturated carbonyl or a hydrogen borrowing reaction.
